(19) United States
(12) Patent Application Publication
(10) Pub. No.: US 2015/0211068 A1
(43) Pub. Date: Jul. 30, 2015

(54) METHODS FOR ASSESSING WHETHER A GENETIC REGION IS ASSOCIATED WITH INFERTILITY

(71) Applicant: CELMATIX, INC., New York, NY (US)

(72) Inventors: Piraye Yurttas Beim, New York, NY (US); Michael Elashoff, Redwood City, CA (US)

(21) Appl. No.: 14/605,440

(22) Filed: Jan. 26, 2015

Related U.S. Application Data
(60) Provisional application No. 61/932,233, filed on Jan. 27, 2014.

(51) Int. Cl. C12Q 1/68; G01N 33/50

(52) U.S. Cl. CPC C12Q 1/6883; G01N 33/5088; C12Q 2600/156; C12Q 2600/124; G01N 2800/367

(57) ABSTRACT

The invention generally relates to methods for assessing whether a genetic region is associated with infertility.

### RELATED APPLICATION

This application claims the benefit of and priority to U.S. Provisional No. 61/932,233, filed Jan. 27, 2014, which is incorporated by reference in its entirety.

### TECHNICAL FIELD

The invention generally relates to methods for assessing whether a genetic region is associated with fecundity and fertility disorders.

### BACKGROUND

Approximately one in seven couples has difficulty conceiving. Infertility may be due to a single cause in either partner, or a combination of factors (e.g., genetic factors, diseases, or environmental factors) that may prevent a pregnancy from occurring or continuing. Every woman will become infertile in her lifetime due to menopause. On average, egg quality and number begins to decline precipitously at 35. However, some women experience this decline much earlier in life, while a number of women are fertile well into their 40s. Similarly, while it is normal for women's reproductive lifespans to include periods of natural infertility, associated with menstrual periods or post-partum changes in reproductive endocrinology, for example, some women experience abnormally extended periods of infertility. Such disorders are referred to as infertility-, fecundity-, or fertility-related disorders. Though, generally, advanced maternal age (35 and above) is associated with poorer fertility outcomes, there is no way of diagnosing egg quality issues in younger women or knowing when a particular woman will start to experience decline in her egg quality or reserve.

The elucidation of the genetic basis of female fecundity and fertility disorders permits the development of powerful, rapid, and non-invasive diagnostic tools that will help clinicians direct patients to efficient and effective treatment options. Additionally, the discovery of the key genetic loci underlying these disorders holds great promise for the identification of novel targets for drug development and therapeutics. Finally, a better understanding of the crucial molecular pathways underlying human fecundity and fertility guides the next generation of targeted, non-hormonal contraceptives.

### SUMMARY

The invention utilizes the status of various fecundity and fertility-related genomic regions in order to assess risk and/or susceptibility to reduced fecundity, fertility, premature menopause, or extended periods of infertility. Methods of the invention utilize genomic information, including, but not limited to, one or more polymorphisms in one or more fecundity- or fertility-related genomic regions, mutations in one or more of those regions, or epigenetic factors affecting expression in those regions. Mutations in a fecundity- or fertility-related genomic region may result in an alternative splicing event, lowered or increased RNA expression, and/or alterations in protein expression, with concomitant physiological changes. Methods of the invention are useful for informing a patient of her susceptibility to abnormally extended periods of infertility or reduced fecundity in connection with age or other relevant phenotypic factors, such as hormone levels or ovarian follicle count.

The invention generally provides methods for assessing whether a genomic region is associated with a fertility-related condition. Aspects of the invention are accomplished using a transgenic animal, such as a genetically-modified mouse. A genomic region suspected to be associated with abnormal fecundity or extended period of infertility is identified. Using that information, the invention provides for genomic modification of a test animal, such as a mouse. The genetically-modified animal is then assessed for the presence of an infertility-associated phenotype. The presence of the phenotype is indicative that the selected genomic region is associated with an infertility-related condition. Methods of the invention allow for the discovery of the key genomic regions underlying fecundity, fertility and infertility and for the subsequent identification of novel targets for drug development and therapeutics. Additionally, genetically-altered test animals that show presence of an infertility phenotype are useful for therapeutic testing.

A genetic locus can encompass a gene and/or upstream and downstream elements, such as introns, promoters and the like, that are involved in the expression of that gene or other genetic loci. There are numerous methods that are useful to identify a genetic locus whose function is suspected of being associated with extended infertility, including reference to literature, databases and empirical analysis. In certain embodiments of the invention, identifying a fertility-related genomic region involves obtaining data on a set of genetic loci, the set including loci known to be associated with infertility and loci having no prior association with infertility. A clustering analysis is then performed on the data to identify genetic loci that have no prior association with infertility that cluster with one or more genetic loci known to be associated with infertility. Thus, genetic loci that have no prior association with infertility are identified as being infertility-related by virtue of clustering with known infertility-related genetic loci. For example, a genetically-altered mouse having a gene knock-out is produced to determine if that gene is implicated in an infertility-associated phenotype. In that manner, genetic loci not previously associated with infertility are identified as potential infertility biomarkers.

Infertility may not be the result of a single genomic alteration, but rather may be the result of a combination of multiple factors or multiple alterations. Methods of the invention provide a better understanding of the molecular pathways underlying human fertility. For example, presence of an infertility-associated phenotype is used as a factor in ranking the importance of a gene in a database of genetic loci associated with infertility in humans by associated the gene (or more often a mutation) with the phenotype. A correlation between the presence of an allele or a mutation in a gene with phenotype increases or decreases the predictive value of the contribution of the genomic region to phenotype.

Additionally, the invention provides genetically altered mice for testing therapeutic agents. In those embodiments, methods of the invention further involve administering a therapeutic agent to the mouse, and assessing the effect of the therapeutic agent on phenotype. A therapeutic agent that rescues the phenotype, i.e., returns or partially re-establishes the wild type fertility phenotype, is a good drug candidate.

Other aspects of the invention provide methods for assessing whether a human genomic alteration is associated with an infertility phenotype in a mouse. Those methods involve identifying a human genomic region whose function is known to be associated with human infertility. The methods additionally involve producing a genetically-modified mouse in which the genetic region whose function is associated with human infertility is altered. The mouse is then assessed for presence of the infertility phenotype.

Other aspects and alternatives for use of the present invention are apparent to the skilled artisan as provided in the detailed description of the invention that follows.

### BRIEF DESCRIPTION OF THE DRAWINGS

FIG. 1 depicts the rate of decline of fertility with age and the corresponding increase in the risk of infertility with age. The shades areas represent different age groups who would benefit from a genetic screen for infertility risk (late teen to mid 40's) versus a genetic screen of premature decline in fertility (late teens to late 30's).

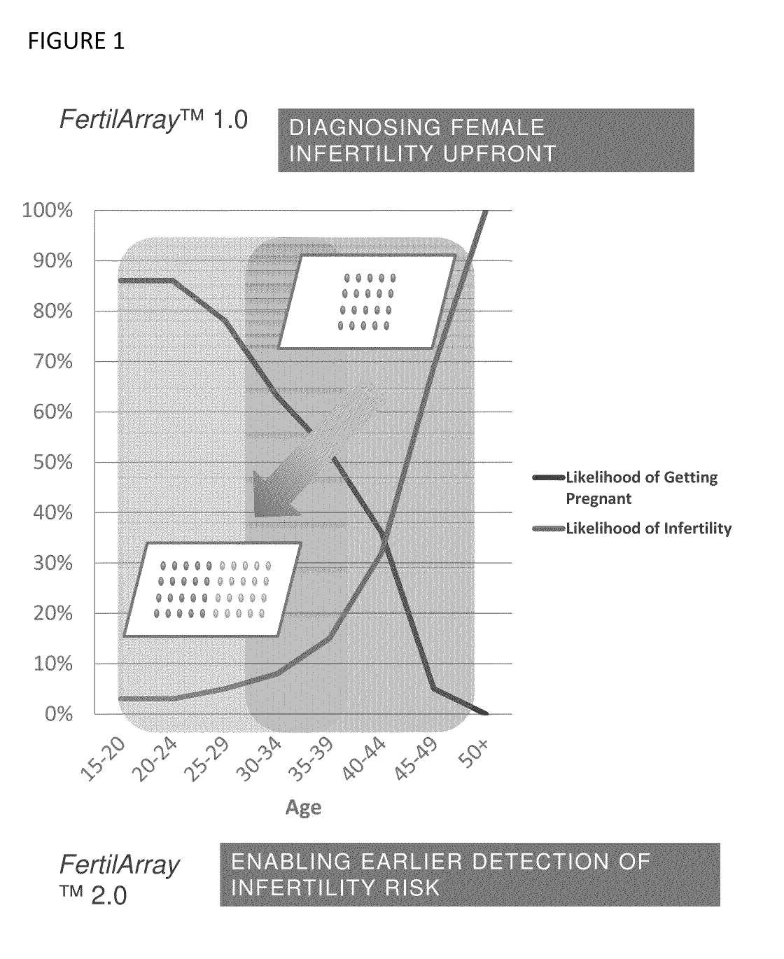

FIG. 2 depicts one way that phenotypic variables can be utilized to accelerate the discovery of genetic regions related to female infertility.

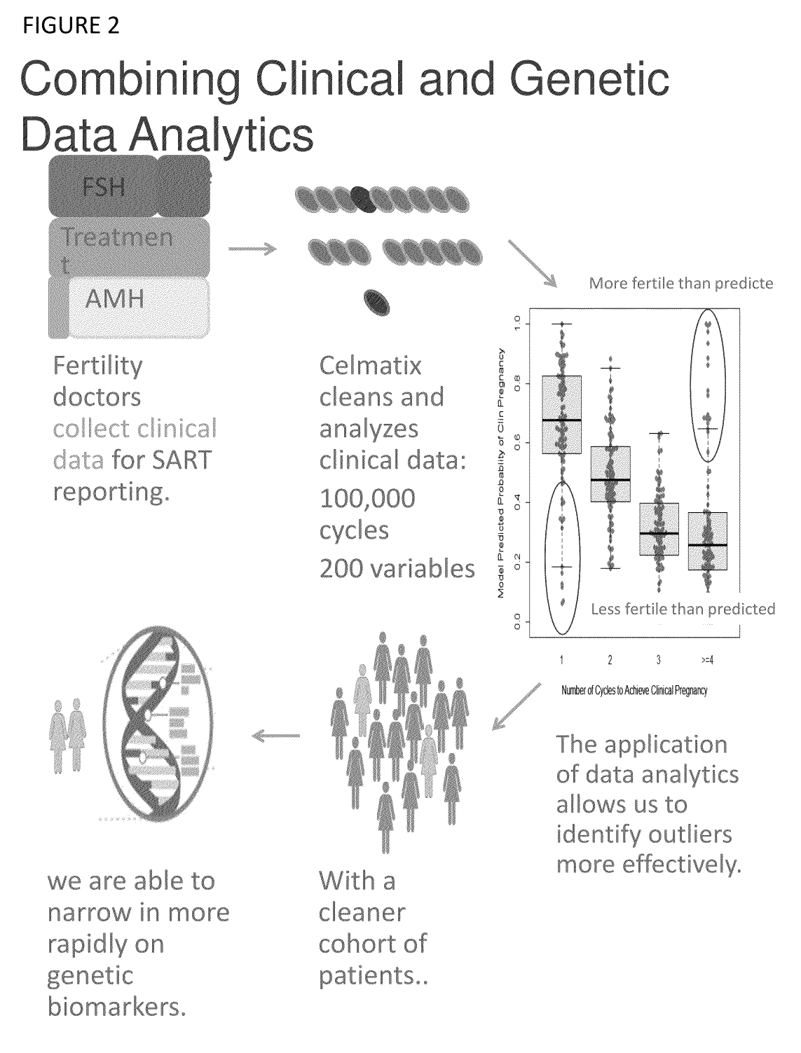

FIG. 3 depicts the methodology for integrating clinical data with genomic data to predict treatment dependent and independent fertility outcomes.

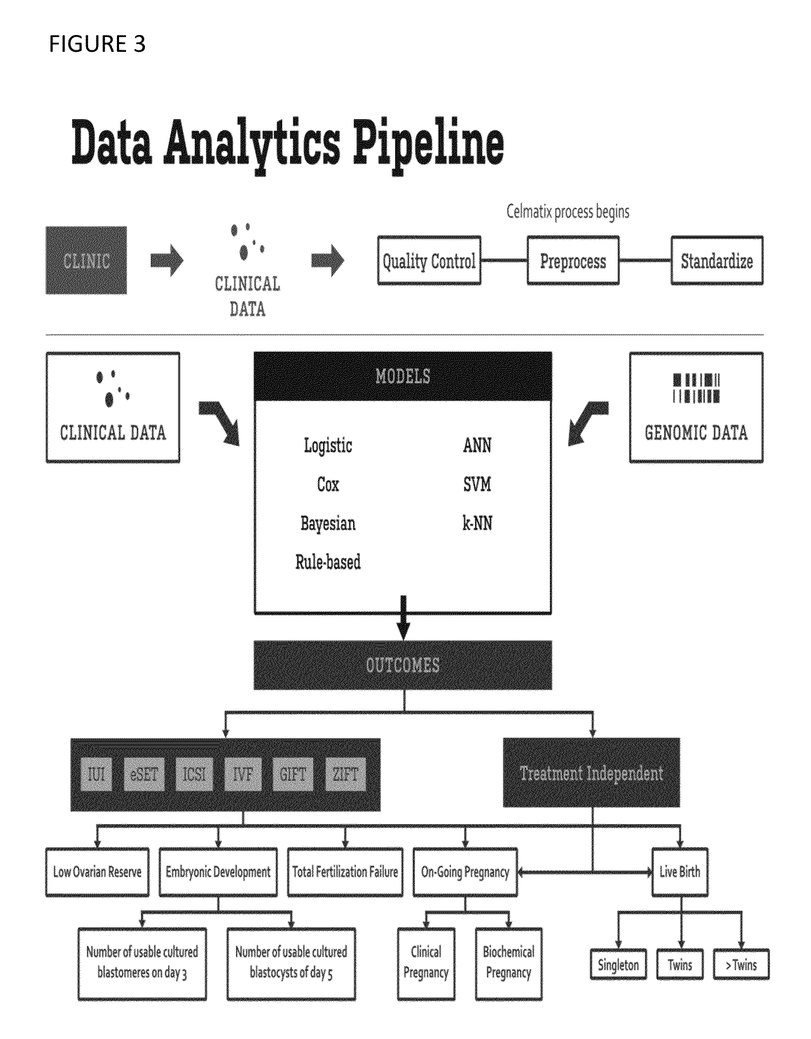

FIG. 4 depicts the different kinds of genetic variants associated with risk of infertility.

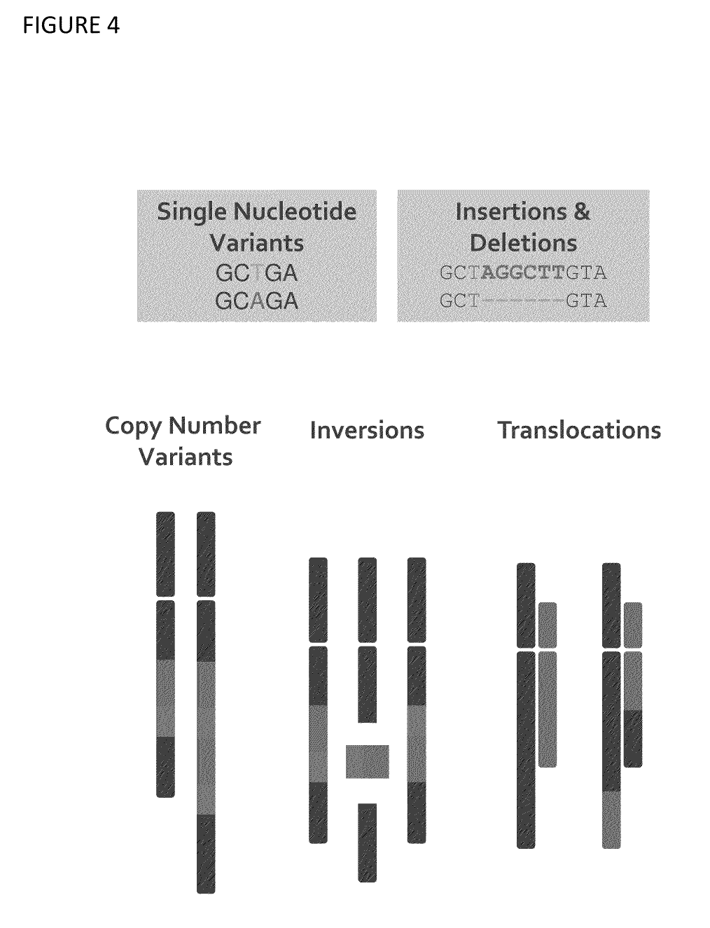

FIG. 5 depicts a method for filtering through variants detected in whole genome sequencing for the identification of genetic regions related to infertility.

FIG. 6 depicts some of the components of the Fertilome™Database, a tool for correlating genetic regions with risk for infertility (Fertilome™Score).

FIG. 7 is the bioinformatics pipeline used to identify biologically interesting and statistically significant genetic variants in infertile patients.

FIG. 8 shows the different types of biologically or statistically significant genetic variants that were detected in infertile patients in the MUC4 genetic region.

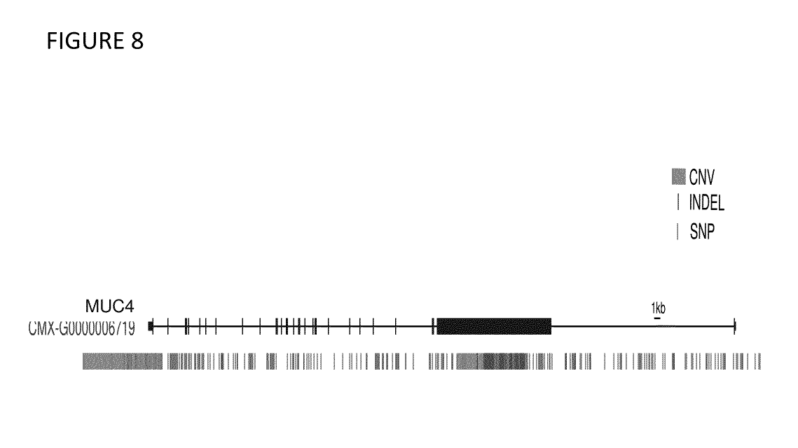

FIG. 9 provides CGH array data of copy number variations associated with infertility.

FIG. 10 illustrates a specific copy number variation detected in the GJC2 gene of Chromosome 1.

FIG. 11 illustrates a specific copy number variation detected in the CRTC1 and GDF1 genes of Chromosome 19.

FIG. 12 illustrates a specific copy number variation detected in a non-coding region of Chromosome 6.

FIG. 13 illustrates population stratification correction of two patient groups (ZA=patients who did not get pregnant with IVF treatment, ZB=patients with infertility who did get pregnant with IVF treatment).

FIG. 14 depicts an area of the cluster analysis results.

FIG. 15 illustrates a system for implementing methods of the invention.

### DETAILED DESCRIPTION

The invention generally relates to methods for the identification and determination of genetic loci and phenotypic characteristics related to infertility in humans and mice to develop a mouse model. Furthermore, the information gained from the present invention may be used in generating a mouse model for therapeutic investigations in infertility in humans. The invention generally relates to data analysis of genetic loci and phenotypes to determine not only the relationship between genetic loci and phenotypic characteristics in a mammalian species, but also to identify genetic loci and corresponding phenotypes that are expressed in both humans and mice. By employing ranking methodologies, biomarkers, or genetic loci, that are expressed in both humans and mice can be determined. The present invention provides a powerful data set to be used in development of a mouse model for therapeutic investigations and strategy development in human infertility.

### Biomarkers

A biomarker generally refers to a molecule that may act as an indicator of a biological state. Biomarkers for use with methods of the invention may be any marker that is associated with infertility. Exemplary biomarkers include genes (e.g., any region of DNA encoding a functional product), genetic regions (e.g., regions including genes and intergenic regions with a particular focus on regions conserved throughout evolution in placental mammals), and gene products (e.g., RNA and protein). In certain embodiments, the biomarker is an infertility-associated genetic region. An infertility-associated genetic region is any DNA sequence in which variation is associated with a change in fertility. Examples of changes in fertility include, but are not limited to, the following: a homozygous mutation of an infertility-associated genetic locus leads to a complete loss of fertility; a homozygous mutation of an infertility-associated genetic locus is incompletely penetrant and leads to reduction in fertility that varies from individual to individual; a heterozygous mutation is completely recessive, having no effect on fertility; and the infertility-associated genetic locus is X-linked, such that a potential defect in fertility depends on whether a non-functional allele of the genetic locus is located on an inactive X chromosome (Barr body) or on an expressed X chromosome.

According to certain aspects, methods of the invention provide for determining infertility genetic regions of interest based on data obtained from public and private fertility/infertility related databases. Infertility/fertility related data may include genetic loci involved in the regulation of implantation, idiopathic infertility genetic loci, polycystic ovary syndrome (PCOS) genetic loci, egg quality genetic loci, endometriosis genetic loci, and premature ovarian failure genetic loci. As described below, the infertility/fertility related data can then be processed using evolutionary conservation to identify genomic regions and variations of interest.

Evolutionary conservation analysis involves, generally, comparing nucleic acid sequences among evolutionary and distantly related genomes to identify similarities and differences between coding and/or non-coding regions across the genomes. The similarity between a region being examined and the related genomes correlates to a degree of conservation. Regions (e.g., coding, non-coding regions, and intergenic regions flanking a gene) that maintain a high degree of similarity across genomes over time are considered highly conserved. Differences between the examined region and regions of related genomes indicate that the examined region has evolved over time. If the examined region is conserved among related genomes, the region is generally considered to exhibit or perform functions that are important for the species (i.e., functionally relevant). This is because genetic abnormalities at functionally important regions are typically harmful to the species, and are phased out over the evolutionary time span. Because functional elements are subject to selection, functional regions tend to evolve at slower rates than nonfunctional regions. A degree of conservation (e.g., degree of similarity between a target genomic region and related genomes) that is considered to be functionally relevant depends on the particular application. For example, a functionally relevant degree of conservation may be 80%, 85%, 90%, 91%, 92%, 93%, 94%, 95%, 96% 97%, 98%, 99%, etc. Regions of genetic loci identified by evolutionary conservation as being functionally relevant can then be used as regions of interest for diagnosing diseases and disorders, such as infertility.

According to certain embodiments, infertility regions of interest are identified by performing evolutionary conservation analysis of one or more genetic loci obtained from infertility and/or fertility-related data. The process of filtering through infertility/fertility related databases using evolutionary conservation, according to the invention, is called the ABCoRE algorithm. For example, nucleic acid data obtained from the infertility/fertility related databases can be compared to distantly related genomes in order to assess conservation of the infertility-related nucleic acid. Regions of the nucleic acid determined to be conserved are classified as infertility regions of interest. In one embodiment, methods of the invention assess conservation of coding regions to determine infertility regions of interest. In another embodiment, methods of the invention assess conservation of non-coding regions to determine infertility regions of interest. In further embodiments, methods of the invention assess conservation of intergenic regions (i.e., a non-coding region flanking a gene) to determine infertility regions of interest. In other embodiments, conservation of both coding and non-coding regions is assessed to determine infertility regions of interest. In any of the above embodiments, coding, non-coding, and intergenic regions may be classified as an infertility region of interest if they have a degree of conservation of, for example, 80%, 85%, 90%, 91%, 92%, 93%, 94%, 95%, 96% 97%, 98%, 99%, etc.

In particular aspects, the following method is employed to determine whether a genomic region is a fertility region of interest using conservation analysis. First, private and/or public nucleic acid data corresponding to infertility or fertility is obtained. Next, one or more genetic loci from that data is examined for conservation. The coding regions (i.e., exons)) of a gene, non-coding regions of the gene, and/or regions flanking the gene (intergenic regions upstream and downstream from the gene being examined) are then analyzed for conservation. According to certain embodiments, if the coding region is found to be conserved (e.g., a degree of conservation 90% or above), the coding region is considered to be an infertility region of interest. The degree of conservation of the non-coding region is then compared to the degree of conservation of the coding region. If the degree of conservation of the non-coding region is similar to the degree of conservation of the coding region, then the non-coding region is also classified an infertility region of interest. This degree of conservation comparison may also be used to determine whether intergenic regions flanking a gene should be classified as an infertility region of interest.

Conservation of coding and/or non-coding sequences is described in Hardison, R. C., Oeltjen, J., and Miller, W. 1997. Long human-mouse sequence alignments reveal novel regulatory elements: A reason to sequence the mouse genome. Genome Res. 7: 959-966; Brenner, S., Venkatesh, B., Yap, W. H., Chou, C. F., Tay, A., Ponniah, S., Wang, Y., and Tan, Y. H. 2002. Conserved regulation of the lymphocyte-specific expression of lck in the Fugu and mammals. Proc. Natl. Acad. Sci. 99: 2936-2941; Karolchik, Donna, et al. “Comparative genomic analysis using the UCSC genome browser.” Comparative Genomics. Humana Press, 2008. 17-33; Santini, Simona, Jeffrey L. Boore, and Axel Meyer. “Evolutionary conservation of regulatory elements in vertebrate Hox gene clusters.” Genome research 13.6a (2003): 1111-1122; Roth, F. P., Hughes, J. D., Estep, P. W., and Church, G. M. 1998. Finding DNA regulatory motifs within unaligned noncoding sequences clustered by whole-genome mRNA quantitation. Nat. Biotechnol. 16: 939-945; and Blanchette, M. and Tompa, M. 2002. Discovery of regulatory elements by a computational method for phylogenetic footprinting. Genome Res. 12: 739-748.

In particular embodiments, the infertility-associated genetic region is a maternal effect gene. Maternal effects genes are genetic loci that have been found to encode key structures and functions in mammalian oocytes (Yurttas et al., Reproduction 139:809-823, 2010). Maternal effect genes are described, for example in, Christians et al. (Mol Cell Biol 17:778-88, 1997); Christians et al., Nature 407:693-694, 2000); Xiao et al. (EMBO J 18:5943-5952, 1999); Tong et al. (Endocrinology 145:1427-1434, 2004); Tong et al. (Nat Genet 26:267-268, 2000); Tong et al. (Endocrinology, 140:3720-3726, 1999); Tong et al. (Hum Reprod 17:903-911, 2002); Ohsugi et al. (Development 135:259-269, 2008); Borowczyk et al. (Proc Natl Acad Sci USA., 2009); and Wu (Hum Reprod 24:415-424, 2009). The content of each of these is incorporated by reference herein in its entirety.

The above-described infertility genetic regions of interest may then be ranked according to significance using one or more the following ranking schemes of the invention.

In particular embodiments, the infertility-associated genetic region is a gene (including exons, introns, and evolutionarily conserved regions of DNA flanking either side of said gene) that impacts fertility selected from the genes shown in Table 1 below. In Table 1, HGNC (http://www.genenames.org/) reference numbers are provided when available.

Table 1 below depicts one possible gene ranking scheme for the relative infertility, subfertility, or premature decline in fertility risk associated with novel or common mutations or variants in a fertility gene. The number of variants column corresponds to the experimental observations of these variants in a study of women with unexplained infertility. The most highly ranked (from top to bottom) genes in this list contained the most variants that were predicted to significantly affect protein structure and function (biologically significant) out of a list of fertility related genes. Genetic variants considered to be biologically significant include mutations that result in a change: 1) to a different amino acid predicted to alter the folding and/or structure of the encoded protein, 2) to a different amino acid occurring at a site with high evolutionarily conservation in mammals, 3) that introduces a premature stop termination signal, 4) that causes a stop termination signal to be lost, 5) that introduces a new start codon, 6) that causes a start codon to be lost, 7) that disrupts a splicing signal, 8) that alters the reading frame or 9) that alters the dosage of encoded protein or RNA. All genetic variants detected from re-sequencing exclude sites where the variant allele is detected in only one chromosome (singletons) and sites sequenced in only one individual.

TABLE 1
Genomic loci containing biologically significant mutations
ranked based on number of biologically significant variants
observed in a study of unexplained female infertility.

| | | | | Number of | |
|---|---|---|---|---|---|
| | Celmatix | Entrez | HGNC | Variants | Variant Description |
| Gene | Gene ID | ID | ID | detected | (type and count) |
| MUC4 | CMX- | 4585 | 7514 | 353 | Drastic |
| | G0000006719 | | | | nonsynonymous: 352; |
| | | | | | Start codon gained: 1 |
| EPHA8 | CMX- | 2046 | 3391 | 23 | CNV loss: 23 |
| | G0000000415 | | | | |
| LOXL4 | GMX- | 84171 | 17171 | 11 | CNV loss: 11 |
| | G0000016263 | | | | |
| FGF8 | CMX- | 2253 | 3686 | 4 | CNV gain: 4 |
| | G0000016316 | | | | |
| KISS1R | CMX- | 84634 | 4510 | 4 | CNV gain: 4 |
| | G0000026560 | | | | |
| SCARB1 | CMX- | 949 | 1664 | 4 | Drastic |
| | G0000019991 | | | | nonsynonymous: 1; |
| | | | | | Start codon gained: 3 |
| BARD1 | CMX- | 580 | 952 | 3 | Drastic |
| | G0000004834 | | | | nonsynonymous: 1; |
| | | | | | Start codon gained: 1; |
| | | | | | Start codon lost: 1 |
| DDX20 | CMX- | 11218 | 2743 | 3 | Start codon gained: 3 |
| | G0000001412 | | | | |
| ECHS1 | CMX- | 1892 | 3151 | 3 | CNV gain: 2; CNV |
| | G0000016S94 | | | | loss: 1 |
| FMN2 | CMX- | 56776 | 14074 | 3 | Start codon gained: 3 |
| | G0000002910 | | | | |
| FOXO3 | CMX- | 2309 | 3821 | 3 | CNV gain: 3 |
| | G0000010672 | | | | |
| HS6ST1 | CMX- | 9394 | 5201 | 3 | Drastic |
| | G0000004221 | | | | nonsynonymous: 3 |
| MAP3K2 | CMX- | 10746 | 6854 | 3 | CNV gain: 3 |
| | G0000004205 | | | | |
| MST1 | CMX- | 4485 | 7380 | 3 | Drastic |
| | G0000005619 | | | | nonsynonymous: 2; |
| | | | | | Splice site acceptor: 1 |
| MTRR | CMX- | 4552 | 7473 | 3 | Drastic |
| | G0000008130 | | | | nonsynonymous: 3 |
| NLRP11 | CMX- | 204801 | 22945 | 3 | Drastic |
| | G0000028188 | | | | nonsynonymous: 2; |
| | | | | | Start codon gained: 1 |
| NLRP14 | CMX- | 338323 | 22939 | 3 | Drastic |
| | G0000016919 | | | | nonsynonymous: 3 |
| NLRP8 | CMX- | 126205 | 22940 | 3 | Drastic |
| | G0000028191 | | | | nonsynonymous: 2; |
| | | | | | Stop codon lost: 1 |
| ASGL2 | CMX- | 430 | 739 | 2 | Start codon gained: 1; |
| | G0000016707 | | | | CNV gain: 1 |
| BMP6 | CMX- | 654 | 1073 | 2 | CNV loss: 2 |
| | G0000009564 | | | | |
| BRCA1 | CMX- | 672 | 1100 | 2 | Drastic |
| | G0000025305 | | | | nonsynonymous: 2 |
| BRCA2 | CMX- | 675 | 1101 | 2 | Drastic |
| | G0000020222 | | | | nonsynonymous: 2 |
| CENPI | CMX- | 2491 | 3968 | 2 | Start codom gained: 2 |
| | G0000031175 | | | | |
| COMT | CMX- | 1312 | 2228 | 2 | Drastic |
| | G0000029621 | | | | nonsynonymous: 1; |
| | | | | | Start codon gained: 1 |
| CYP11B1 | CMX- | 1584 | 2591 | 2 | CNV gain: 2 |
| | G0000013888 | | | | |
| DAZL | CMX- | 1618 | 2685 | 2 | Start codon gained: 2 |
| | G0000005296 | | | | |
| EEF1A1 | CMX- | 1915 | 3189 | 2 | Start codon gained: 2 |
| | G0000010487 | | | | |
| FMR1 | CMX- | 2332 | 3775 | 2 | Drastic |
| | G0000031614 | | | | nonsynonymous: 1; |
| | | | | | Start codon gained: 1 |
| GDF1 | CMX- | 2657 | 4214 | 2 | Drastic |
| | G0000027183 | | | | nonsynonymous: 1; |
| | | | | | CNV gain: 1 |
| HK3 | CMX- | 3101 | 4925 | 2 | Drastic |
| | G0000009361 | | | | nonsynonymous: 2 |
| IGF2 | CMX- | 3481 | 5466 | 2 | CNV gain: 2 |
| | G0000016702 | | | | |
| ISG15 | CMX- | 9636 | 4053 | 2 | CNV gain: 2 |
| | G0000000029 | | | | |
| JMY | CMX- | 133746 | 28916 | 2 | Drastic |
| | G0000008593 | | | | nonsynonymous: 2 |
| KL | CMX- | 9365 | 6344 | 2 | Drastic |
| | G0000020228 | | | | nonsynonymous: 2 |
| MTHFR | CMX- | 4524 | 7436 | 2 | Drastic |
| | G0000000213 | | | | nonsynonymous: 1; |
| | | | | | Start codon gained: 1 |
| NLRP13 | CMX- | 126204 | 22937 | 1 | Drastic |
| | G0000028190 | | | | nonsynonymous: 2 |
| MLRP5 | CMX- | 126206 | 21269 | 2 | Drastic |
| | G0000028192 | | | | nonsynonymous: 2 |
| NOBOX | CMX- | 135935 | 22448 | 2 | Drastic |
| | G0000012690 | | | | nonsynonymous: 2 |
| PRKRA | CMX- | 8575 | 9438 | 2 | Drastic |
| | G0000004587 | | | | nonsynonymous: 1; |
| | | | | | Nonsynonymous |
| | | | | | start: 1 |
| SDC3 | CMX- | 9672 | 10660 | 2 | Drastic |
| | G0000000574 | | | | nonsynonymous: 2 |
| TACC3 | CMX- | 10460 | 11524 | 2 | Drastic |
| | G0000006818 | | | | nonsynonymous: 2 |
| TLE6 | CMX- | 79816 | 30788 | 2 | CNV loss: 2 |
| | G0000026639 | | | | |
| ACVR1C | CMX- | 130399 | 18123 | 1 | Drastic |
| | G0000004406 | | | | nonsynonymous: 1 |
| AHR | CMX- | 196 | 348 | 1 | Start codon gained: 1 |
| | G0000011332 | | | | |
| APOA1 | CMX- | 335 | 600 | 1 | CNV gain: 1 |
| | G0000018327 | | | | |
| AURKA | CMX- | 6790 | 11393 | 1 | Start codon gained: 1 |
| | G0000028967 | | | | |
| BMP15 | CMX- | 9210 | 1068 | 1 | CNV gain: 1 |
| | G0000030783 | | | | |
| BMP4 | CMX- | 652 | 1071 | 1 | Stop codon lost: 1 |
| | G0000021216 | | | | |
| C6orf221 | CMX- | 154288 | 33699 | 1 | Drastic |
| | G0000010478 | | | | nonsynonymous: 1 |
| CASP8 | CMX- | 841 | 1509 | 1 | CNV loss: 1 |
| | G0000004721 | | | | |
| CBS | CMX- | 875 | 1550 | 1 | Drastic |
| | G0000029408 | | | | nonsynonymous: 1 |
| CDX2 | CMX- | 1045 | 1806 | 1 | Drastic |
| | G0000020191 | | | | nonsynonymous: 1 |
| CENPF | CMX- | 1063 | 1857 | 1 | Drastic |
| | G0000002670 | | | | nonsynonymous: 1 |
| CGB | CMX- | 1082 | 1886 | 1 | Start codon gained: 1 |
| | G0000027860 | | | | |
| CSF1 | CMX- | 1435 | 2432 | 1 | CNV loss: 1 |
| | G0000001574 | | | | |
| CSF2 | CMX- | 1437 | 2434 | 1 | CNV loss: 1 |
| | G0000008885 | | | | |
| BCTPP1 | CMX- | 79077 | 28777 | 1 | CNV gain: 1 |
| | G0000023705 | | | | |
| DNMT1 | CMX- | 1786 | 2976 | 1 | Drastic |
| | G0000026880 | | | | nonsynonymous: 1 |
| EFNA4 | CMX- | 1945 | 3224 | 1 | CNV loss: 1 |
| | G0000001896 | | | | |
| EFNB3 | CMX- | 1949 | 3228 | 1 | CNV gain: 1 |
| | G0000024616 | | | | |
| EIF3CL | CMX- | 728689 | 26347 | 1 | CNV loss: 1 |
| | G0000023621 | | | | |
| EPHA5 | CMX- | 2044 | 3389 | 1 | CNV loss: 1 |
| | G0000007213 | | | | |
| EPHA7 | CMX- | 2045 | 3390 | 1 | CNV loss: 1 |
| | G0000010603 | | | | |
| EZH2 | CMX- | 2146 | 3527 | 1 | Drastic |
| | G0000012702 | | | | nonsynonymous: 1 |
| FOXL2 | CMX- | 668 | 1092 | 1 | Start codon gained: 1 |
| | G0000006297 | | | | |
| FOXP3 | CMX- | 50943 | 6106 | 1 | CNV gain: 1 |
| | G0000030750 | | | | |
| GALT | CMX- | 2592 | 4135 | 1 | Splice site acceptor: 1 |
| | G0000014248 | | | | |
| GDF9 | CMX- | 2661 | 4224 | 1 | Start codon gained: 1 |
| | G0000008902 | | | | |
| GJA4 | CMX- | 2701 | 4278 | 1 | CNV gain: 1 |
| | G0000000643 | | | | |
| GJB3 | CMX- | 2707 | 4285 | 1 | CNV gain: 1 |
| | G0000000642 | | | | |
| GJB4 | CMX- | 127534 | 4286 | 1 | CNV gain: 1 |
| | G0000000641 | | | | |
| GJD3 | CMX- | 125111 | 19147 | 1 | CNV gain: 1 |
| | G0000025169 | | | | |
| GPC3 | CMX- | 2719 | 4451 | 1 | CNV gain: 1 |
| | G0000031486 | | | | |
| HSD17B2 | CMX- | 3294 | 5211 | 1 | Drastic |
| | G0000024260 | | | | nonsynonymous: 1 |
| IGFBPL1 | CMX- | 347252 | 20081 | 1 | CNV loss: 1 |
| | G0000014341 | | | | |
| KISS1 | CMX- | 3814 | 6341 | 1 | CNV gain: 1 |
| | G0000002533 | | | | |
| LHCGR | CMX- | 3973 | 6585 | 1 | Drastic |
| | G0000003462 | | | | nonsynonymous: 1 |
| MAD1L1 | CMX- | 8379 | 6762 | 1 | Start codon gained: 1 |
| | G0000011200 | | | | |
| MAB2L1 | CMX- | 4085 | 6763 | 1 | Start codon gained: 1 |
| | G0000007650 | | | | |
| MB21D1 | CMX- | 115004 | 21367 | 1 | Drastic |
| | G0000010484 | | | | nonsynonymous: 1 |
| MCM8 | CMX- | 84515 | 16147 | 1 | Drastic |
| | G0000028433 | | | | nonsynonymous: 1 |
| MYC | CMX- | 4609 | 7553 | 1 | Start codon gained: 1 |
| | G0000013826 | | | | |
| HLRP2 | CMX- | 55655 | 22948 | 1 | Start codon gained: 1 |
| | G0000028I40 | | | | |
| NLRP4 | CMX- | 147945 | 22943 | 1 | Start codon gained: 1 |
| | G0000028189 | | | | |
| OAS1 | CMX- | 4938 | 8086 | 1 | Splice site acceptor: 1 |
| | G0000019838 | | | | |
| PADI3 | CMX- | 51702 | 18337 | 1 | CNV gain: 1 |
| | G0000000342 | | | | |
| PAEP | CMX- | 5047 | 8573 | 1 | CNV gain: 1 |
| | G0000015254 | | | | |
| PLCB1 | CMX- | 23236 | 15917 | 1 | CNV gain: 1 |
| | G0000028445 | | | | |
| PMS2 | CMX- | 5395 | 9122 | 1 | Drastic |
| | G0000011251 | | | | nonsynonymous: 1 |
| POF1B | CMX- | 79983 | 13711 | 1 | CNV gain: 1 |
| | G0000031099 | | | | |
| PRDM9 | CMX- | 56979 | 13994 | 1 | CNV loss: 1 |
| | G0000008219 | | | | |
| SEPHS2 | CMX- | 22928 | 19686 | 1 | CNV gain: 1 |
| | G0000023707 | | | | |
| SERPINA10 | CMX- | 51156 | 15996 | 1 | CNV gain: 1 |
| | G0000021629 | | | | |
| SIRT3 | CMX- | 23410 | 14931 | 1 | CNV loss: 1 |
| | G0000016629 | | | | |
| SPN | CMX- | 101929889 | 11249 | 1 | CNV loss: 1 |
| | G0000023664 | | | | |
| TFPI | CMX- | 7035 | 11760 | 1 | Drastic |
| | G0000004632 | | | | nonsynonymous: 1 |
| TGFB1I1 | CMX- | 7041 | 11767 | 1 | CNV gain: 1 |
| | G0000023757 | | | | |
| TP63 | CMX- | 8626 | 15979 | 1 | Start codon gained: 1 |
| | G0000006674 | | | | |
| UBE3A | CMX- | 7337 | 12496 | 1 | Start codon gained: 1 |
| | G0000022200 | | | | |
| UBL4B | CMX- | 164153 | 32309 | 1 | CNV loss: 1 |
| | G0000001378 | | | | |
| UIMC1 | CMX- | 51720 | 30298 | 1 | Drastic |
| | G0000009362 | | | | nonsynonymous: 1 |
| VKORC1 | CMX- | 79001 | 23663 | 1 | CNV gain: 1 |
| | G0000023741 | | | | |
| ZF3 | CMX- | 7784 | 13189 | 1 | Start codon gained: 1 |
| | G0000011947 | | | | |

In particular embodiments, the infertility-associated genetic region is a gene (including exons, introns, and evolutionarily conserved regions of DNA flanking either side of said gene) that impacts fertility selected from the genes shown in Table 2 below. In Table 2, HGNC (http://www.genenames.org/) reference numbers are provided when available.

Table 2 below depicts another possible gene ranking scheme for the relative infertility, subfertility, or premature decline in fertility risk associated with novel or common mutations or variants in a fertility gene. Table 2 contains the 10 genes, listed in order from most to least statistically significant, that were determined to be statistically significantly correlated with infertility risk in a study of unexplained female infertilty based on variants detected in the coding regions of these genes. P-values<0.025 are considered statistically significant, and all other fertility genes did not fit the pass the significance test for inclusion and ranking in this list. For the coding level analysis, we first compute a coding variant score for the coding regions for each individual/gene. The coding variant score represents the variability of the gene at coding regions in an individual and is computed as the sum of the proportion of variant locations within the coding regions of that gene for that individual. A series of linear regression models are fit, where the outcome variable is the coding variant score for a given gene, and the independent variables are group (infertile vs control) and principal component derived ethnicity (continuous). The p-value for group is used for statistical inference. The model is fit once for each gene.

TABLE 2
Fertility genes demonstrating statistical significance at the gene
coding region level for infertility risk ranked based on p-values,
observed in a study of unexplained female infertility.

| Gene | Celmatix Gene ID | Entrez ID | HGNC ID | P-value |
|---|---|---|---|---|
| ZF4 | CMX-G0000002903 | 57829 | 15770 | 5.17E−10 |
| UIMC1 | CMX-G0000009362 | 51720 | 30298 | 0.001401803 |
| PAD16 | CMX-G0000000344 | 353238 | 20449 | 0.003420271 |
| ZP1 | CMX-G0000017558 | 22917 | 13187 | 0.003845858 |
| MDM2 | CMX-G0000019503 | 4193 | 6973 | 0.009323844 |
| PRKRA | CMX-G0000004587 | 8575 | 9438 | 0.009832035 |
| PMS2 | CMX-G0000011251 | 5395 | 9122 | 0.015453858 |
| TGFB1 | CMX-G0000027588 | 7040 | 11766 | 0.018576967 |
| ESR2 | CMX-G0000021326 | 2100 | 3468 | 0.022661688 |
| PRDM1 | CMX-G0000010653 | 639 | 9346 | 0.024522163 |

In particular embodiments, the infertility-associated genetic region is a gene (including exons, introns, and evolutionarily conserved regions of DNA flanking either side of said gene) that impacts fertility selected from the genes shown in Table 3 below. In Table 3, HGNC (http://www.genenames.org/) reference numbers are provided when available.

Table 3 below depicts another possible gene ranking scheme for the relative infertility, subfertility, or premature decline in fertility risk associated with novel or common mutations or variants in a fertility gene. Table 3 contains the 11 genes, listed in order from most to least statistically significant, that were determined to be statistically significantly correlated with infertility risk in a study of unexplained female infertilty based on variants detected in the coding, non-coding, and conserved upstream and downstream regions of the fertility gene. P-values<0.025 are considered statistically significant, and all other fertility genes did not fit the pass the significance test for inclusion and ranking in this list. For the gene level analysis, we first compute a gene variant score for the entire transcript and flanking evolutionarily conserved regions for each individual/gene. The gene variant score represents the variability of the gene in an individual and is computed as the sum of the proportion of variant locations within that gene and its evolutionarily conserved regions flanking the gene for that individual. A series of linear regression models are fit, where the outcome variable is the gene variant score for a given gene, and the independent variables are group (infertile vs control) and principal component derived ethnicity (continuous). The p-value for group is used for statistical inference. The model is fit once for each gene.

TABLE 3
Fertility genes demonstrating statistical significance at the
entire gene level for infertility risk ranked based on p-values,
observed in a study of unexplained female infertility.

| Gene | Celmatix Gene ID | Entrez ID | HGNC ID | P-value |
|---|---|---|---|---|
| PADI6 | CMX-G0000000344 | 353238 | 20449 | 0.00079599 |
| CGB | CMX-G0000027860 | 1082 | 1886 | 0.000983714 |
| PMS2 | CMX-G0000011251 | 5395 | 9122 | 0.001500248 |
| ESR2 | CMX-G0000021326 | 2100 | 3468 | 0.004733531 |
| UIMC1 | CMX-G0000009362 | 51720 | 30298 | 0.005170633 |
| ZP1 | CMX-G0000017558 | 22917 | 13187 | 0.00852914 |
| MDM2 | CMX-G0000019503 | 4193 | 6973 | 0.009794758 |
| BRCA2 | CMX-G0000020222 | 675 | 1101 | 0.019744499 |
| TGFB1 | CMX-G0000027588 | 7040 | 11766 | 0.020358934 |
| CDKN1C | CMX-G0000016717 | 1028 | 1786 | 0.022605239 |
| TAF4B | CMX-G0000026229 | 6875 | 11538 | 0.024673723 |

In particular embodiments, the infertility-associated genetic region is a gene (including exons, introns, and evolutionarily conserved regions of DNA flanking either side of said gene) that impacts fertility selected from the genes shown in Table 4 below. In Table 4, HGNC (http://www.genenames.org/) reference numbers are provided when available.

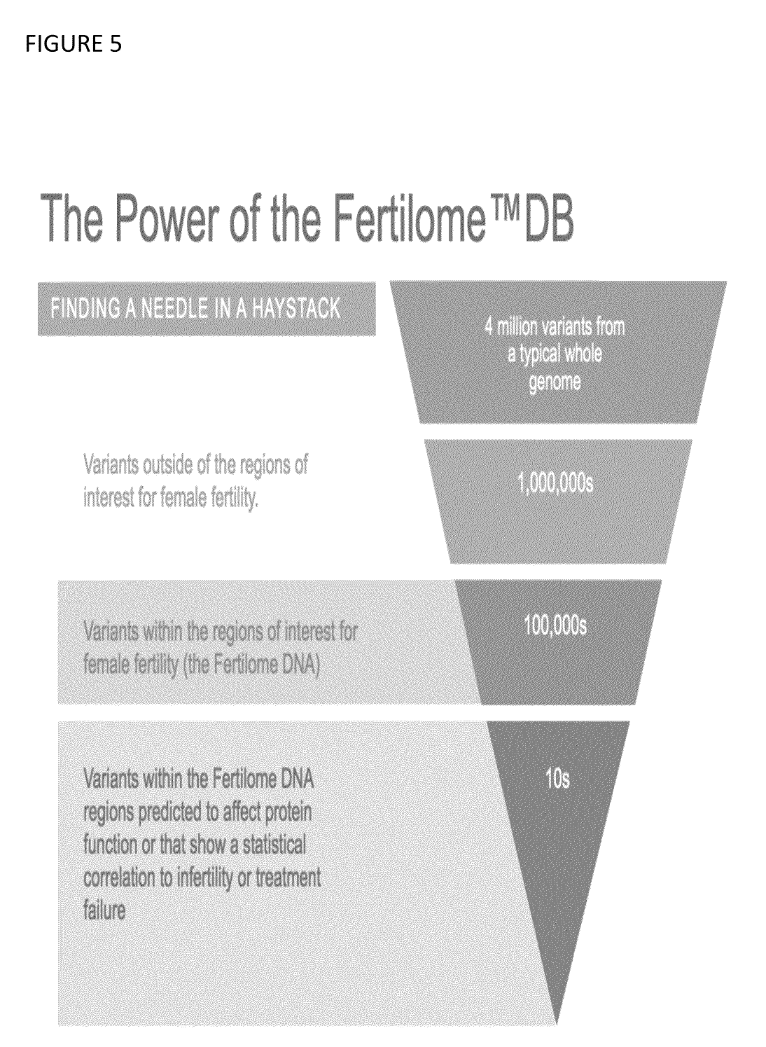

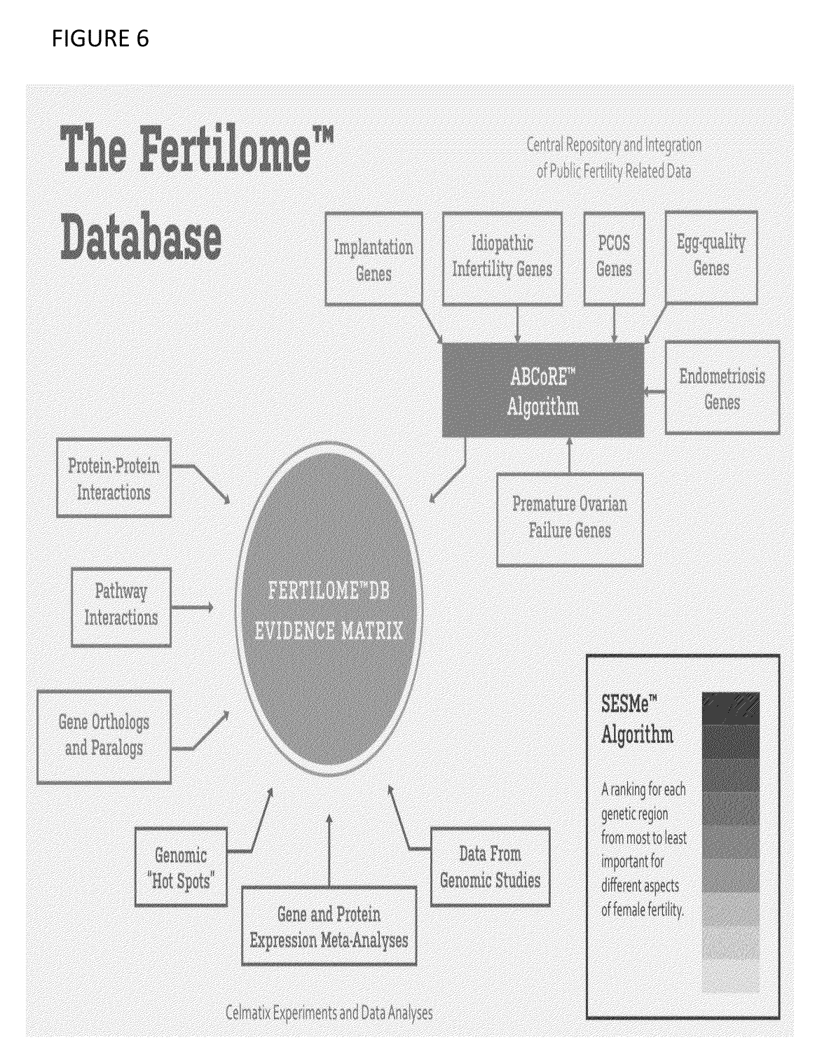

Table 4 below depicts another possible gene ranking scheme for the relative infertility, subfertility, or premature decline in fertility risk associated with novel or common mutations or variants in a fertility gene. Table 4 contains the top ranked 100 fertility genes, listed in order from most to least likely for variants in that gene to affect fertility. Genes are ranked according to a Celmatix Fertilome™Score, G1Version2, that reflects the likelihood a gene is involved in fertility or reproduction. This score is computed using a database of mined and curated data, containing attributes for each gene in the genome (See FIGS. 5 and 6). These attributes include: diseases and disorders related to infertility, molecular pathways, molecular interactions, gene clusters, mouse phenotypes associated with each gene, gene expression data in reproductive tissues, proteomics data in oocytes, and accrued information from scientific publications through text-mining.

The process for ranking fertility-related attributes of a gene or genetic region (locus) to obtain an infertility score is called the SESMe algorithm. The SESMe algorithm is applied to a database of features and attributes that might make a particular gene important for fertility. The algorithm assigns a score and a relative weight to each feature then ranks genetic regions from most to least important (or vice versa) by weighting features and attributes associated with that genetic region. For example, a score is assigned to a gene by compiling the combined weighted values of attributes associated with that gene. After each gene is scored based on its weighted attributes, the genetic loci can be ranked in order of importance in accordance with their score. The weighted value for each infertility attribute may be scaled in any manner including and not limited to assigning a positive or negative integer to reflect the significance or severity of the attribute to infertility.

In certain embodiments, the weighted value for gene infertility attributes may be on a scale from −10 to +10. A+10 may indicate that an attribute of a gene being scored is highly associated with infertility because that attribute is prevalently found in infertile patient populations. A+4 may represent an attribute that is a latent infertility marker, meaning it will not cause infertility on its own, but may lead to infertility upon influence of external factors such as aging and smoking. Whereas +2 may represent an attribute found in some infertile patients but nothing directly relates the attribute to infertility. A zero on the scale may include an attribute not yet known to have any effect or any negative effect towards infertility. A −10 may include an attribute shown not to affect infertility whatsoever. Further, embodiments provide for the weighted scale to include a +1 for attributes that are commonly found in infertile patient populations, 0.5 for attributes similar to those found in infertile patient populations, and 0 for attributes without a causal link to infertility.

In addition, weighted values for attributes may be normalized based on the known significance of that attribute towards infertility. For example and in certain embodiments, when scoring attributes of a particular gene, each attribute may be assigned a 0 if the attribute is absent and a 1 if the attribute is present. The attributes may then be normalized based on the infertility significance of that attribute. For example, if the attribute is a genetic mutation known to be associated with infertility, then that attribute may be normalized by a factor of 5. In another example, if the attribute is a signaling pathway defect sometimes associated with infertility, then that attribute may be normalized by a factor of 2.

Table 4, provided below, lists 100 Human Fertility Genes that were ranked by weighing attributes associated with the gene in accordance with methods of the invention.

TABLE 4
List of Top 100 Human Fertility Genes based on
the Fertilome ™Score, G1Version2.

| | | | Entrez | HGNC | Celmatix |
|---|---|---|---|---|---|
| | Gene | Celmatix | Gene | Gene | Fertilome ™ |
| Rank | Symbol | Gene ID | ID | ID | Score |
| 1 | C6orf221 | CMX- | 154288 | 33699 | 15 |
| | | G0000010478 | | | |
| 2 | NLRP5 | CMX- | 126206 | 21269 | 15 |
| | | G0000028192 | | | |
| 3 | ZP3 | CMX- | 7784 | 13189 | 12.93 |
| | | G0000011947 | | | |
| 4 | FIGLA | CMX- | 344018 | 24669 | 12 |
| | | G0000003616 | | | |
| 5 | PADI6 | CMX- | 353238 | 20449 | 12 |
| | | G0000000344 | | | |
| 6 | DNMT1 | CMX- | 1786 | 2976 | 11.67 |
| | | G0000026880 | | | |
| 7 | ZP2 | CMX- | 7783 | 13188 | 11.67 |
| | | G0000023549 | | | |
| 8 | FSHR | CMX- | 2492 | 3969 | 11.37 |
| | | G0000003464 | | | |
| 9 | OOEP | CMX- | 441161 | 21382 | 11 |
| | | G0000010479 | | | |
| 10 | FOXO3 | CMX- | 2309 | 3821 | 10.39 |
| | | G0000010672 | | | |
| 11 | ACYR1B | CMX- | 91 | 172 | 10.14 |
| | | G0000019186 | | | |
| 12 | CGA | CMX- | 1081 | 1885 | 10.04 |
| | | G0000010560 | | | |
| 13 | INHA | CMX- | 3623 | 6065 | 10.02 |
| | | G0000004914 | | | |
| 14 | LHCGR | CMX- | 3973 | 6585 | 10.01 |
| | | G0000003462 | | | |
| 15 | DPPA3 | CMX- | 359787 | 19199 | 10 |
| | | G0000018719 | | | |
| 16 | KDM1B | CMX- | 221656 | 21577 | 10 |
| | | G0000009642 | | | |
| 17 | NOBOX | CMX- | 135935 | 22448 | 10 |
| | | G0000012690 | | | |
| 18 | NPM2 | CMX- | 10361 | 7930 | 10 |
| | | G0000013114 | | | |
| 19 | ESR1 | CMX- | 2099 | 3467 | 9.91 |
| | | G0000011002 | | | |
| 20 | AURKA | CMX- | 6790 | 11393 | 9.84 |
| | | G0000028967 | | | |
| 21 | BRCA2 | CMX- | 675 | 1101 | 9.75 |
| | | G0000020222 | | | |
| 22 | WT1 | CMX- | 7490 | 12796 | 9.53 |
| | | G0000017126 | | | |
| 23 | CBS | CMX- | 875 | 1550 | 9.49 |
| | | G0000029408 | | | |
| 24 | CDKN1C | CMX- | 1028 | 1786 | 9.37 |
| | | G0000016717 | | | |
| 25 | IGF1 | CMX- | 3479 | 5464 | 9.35 |
| | | G0000019714 | | | |
| 26 | HAND2 | CMX- | 9464 | 4808 | 9.17 |
| | | G0000007954 | | | |
| 27 | GDF9 | CMX- | 2661 | 4224 | 9 |
| | | G0000008902 | | | |
| 28 | MAD2L1 | CMX- | 4085 | 6763 | 9 |
| | | G0000007650 | | | |
| 29 | ZAR1 | CMX- | 326340 | 20436 | 9 |
| | | G0000007128 | | | |
| 30 | FOXL2 | CMX- | 668 | 1092 | 8.88 |
| | | G0000006297 | | | |
| 31 | BARD1 | CMX- | 580 | 952 | 8.54 |
| | | G0000004834 | | | |
| 32 | FMN2 | CMX- | 56776 | 14074 | 8.4 |
| | | G0000002910 | | | |
| 33 | TACC3 | CMX- | 10460 | 11524 | 8.39 |
| | | G0000006818 | | | |
| 34 | MYC | CMX- | 4609 | 7553 | 8.25 |
| | | G0000013826 | | | |
| 35 | IL11RA | CMX- | 3590 | 5967 | 7.9 |
| | | G0000014249 | | | |
| 36 | MCM8 | CMX- | 84515 | 16147 | 7.85 |
| | | G0000028433 | | | |
| 37 | LHB | CMX- | 3972 | 6584 | 7.82 |
| | | G0000027859 | | | |
| 38 | TAF4B | CMX- | 6875 | 11538 | 7.68 |
| | | G0000026229 | | | |
| 39 | USP9X | CMX- | 8239 | 12632 | 7.67 |
| | | G0000030612 | | | |
| 40 | PRLR | CMX- | 5618 | 9446 | 7.58 |
| | | G0000008271 | | | |
| 41 | HSF1 | CMX- | 3297 | 5224 | 7.35 |
| | | G0000013948 | | | |
| 42 | FSHB | CMX- | 2488 | 3964 | 7.33 |
| | | G0000017113 | | | |
| 43 | ZP1 | CMX- | 22917 | 13187 | 7.29 |
| | | G0000017558 | | | |
| 44 | MDM2 | CMX- | 4193 | 6973 | 7.27 |
| | | G0000019503 | | | |
| 45 | BMP15 | CMX- | 9210 | 1068 | 7.25 |
| | | G0000030783 | | | |
| 46 | GPC3 | CMX- | 2719 | 4451 | 7.11 |
| | | G0000031486 | | | |
| 47 | PRDM1 | CMX- | 639 | 9346 | 7.05 |
| | | G0000010653 | | | |
| 48 | FST | CMX- | 10468 | 3971 | 7 |
| | | G0000008371 | | | |
| 49 | EZH2 | CMX- | 2146 | 3527 | 6.91 |
| | | G0000012702 | | | |
| 50 | SMAD2 | CMX- | 4087 | 6768 | 6.89 |
| | | G0000026329 | | | |
| 51 | NODAL | CMX- | 4838 | 7865 | 6.88 |
| | | G0000015959 | | | |
| 52 | ACVR1 | CMX- | 90 | 171 | 6.81 |
| | | G0000004407 | | | |
| 53 | HSD17B12 | CMX- | 51144 | 18646 | 6.71 |
| | | G0000017190 | | | |
| 54 | BRCA1 | CMX- | 672 | 1100 | 6.67 |
| | | G0000025305 | | | |
| 55 | DICER1 | CMX- | 23405 | 17098 | 6.53 |
| | | G0000021645 | | | |
| 56 | ESR2 | CMX- | 2100 | 3468 | 6.47 |
| | | G0000021326 | | | |
| 57 | MDM4 | CMX- | 4194 | 6974 | 6.42 |
| | | G0000002542 | | | |
| 58 | AR | CMX- | 367 | 644 | 6.41 |
| | | G0000030935 | | | |
| 59 | SCARB1 | CMX- | 949 | 1664 | 6.39 |
| | | G0000019991 | | | |
| 60 | CDKN1B | CMX- | 1027 | 1785 | 6.25 |
| | | G0000018846 | | | |
| 61 | TP53 | CMX- | 7157 | 11998 | 6.23 |
| | | G0000024614 | | | |
| 62 | NOG | CMX- | 9241 | 7866 | 6.22 |
| | | G0000025542 | | | |
| 63 | IL6ST | CMX- | 3572 | 6021 | 6.13 |
| | | G0000008398 | | | |
| 64 | DAZL | CMX- | 1618 | 2685 | 6 |
| | | G0000005296 | | | |
| 65 | NLRP11 | CMX- | 204801 | 22945 | 6 |
| | | G0000028188 | | | |
| 66 | NLRP13 | CMX- | 126204 | 22937 | 6 |
| | | G0000028190 | | | |
| 67 | NLRP8 | CMX- | 126205 | 22940 | 6 |
| | | G0000028191 | | | |
| 68 | NLRP9 | CMX- | 338321 | 22941 | 6 |
| | | G0000028184 | | | |
| 69 | ZFX | CMX- | 7543 | 12869 | 5.67 |
| | | G0000030503 | | | |
| 70 | TFPI | CMX- | 7035 | 11760 | 5.36 |
| | | G0000004632 | | | |
| 71 | HSD17B7 | CMX- | 51478 | 5215 | 5.32 |
| | | G0000002148 | | | |
| 72 | TP63 | CMX- | 8626 | 15979 | 5.28 |
| | | G0000006674 | | | |
| 73 | NR5A1 | CMX- | 2516 | 7983 | 5.24 |
| | | G0000015051 | | | |
| 74 | BMP7 | CMX- | 655 | 1074 | 5.09 |
| | | G0000028985 | | | |
| 75 | CGB | CMX- | 1082 | 1886 | 5 |
| | | G0000027860 | | | |
| 76 | CGB5 | CMX- | 93659 | 16452 | 5 |
| | | G0000027866 | | | |
| 77 | DDX43 | CMX- | 55510 | 18677 | 5 |
| | | G0000010483 | | | |
| 78 | FMR1 | CMX- | 2332 | 3775 | 5 |
| | | G0000031614 | | | |
| 79 | LIN28B | CMX- | 389421 | 32207 | 5 |
| | | G0000010647 | | | |
| 80 | NLRP14 | CMX- | 338323 | 22939 | 5 |
| | | G0000016919 | | | |
| 81 | NLRP4 | CMX- | 147945 | 22943 | 5 |
| | | G0000028189 | | | |
| 82 | NLRP7 | CMX- | 199713 | 22947 | 5 |
| | | G0000028139 | | | |
| 83 | PROK1 | CMX- | 84432 | 18454 | 5 |
| | | G0000001385 | | | |
| 84 | SPIN1 | CMX- | 10927 | 11243 | 5 |
| | | G0000014689 | | | |
| 85 | TFPI2 | CMX- | 7980 | 11761 | 5 |
| | | G0000012044 | | | |
| 86 | ZP4 | CMX- | 57829 | 15770 | 5 |
| | | G0000002903 | | | |
| 87 | ESRRB | CMX- | 2103 | 3473 | 4.8 |
| | | G0000021489 | | | |
| 88 | UBE3A | CMX- | 7337 | 12496 | 4.76 |
| | | G0000022200 | | | |
| 89 | SUZ12 | CMX- | 23512 | 17101 | 4.73 |
| | | G0000025003 | | | |
| 90 | XIST | CMX- | 7503 | 12810 | 4.7 |
| | | G0000031023 | | | |
| 91 | ATM | CMX- | 472 | 795 | 4.62 |
| | | G0000018234 | | | |
| 92 | AURKB | CMX- | 9212 | 11390 | 4.55 |
| | | G0000024639 | | | |
| 93 | STK3 | CMX- | 6788 | 11406 | 4.52 |
| | | G0000013673 | | | |
| 94 | POLG | CMX- | 5428 | 9179 | 4.51 |
| | | G0000023009 | | | |
| 95 | CDX2 | CMX- | 1045 | 1806 | 4.46 |
| | | G0000020191 | | | |
| 96 | TP73 | CMX- | 7161 | 12003 | 4.43 |
| | | G0000000110 | | | |
| 97 | MTOR | CMX- | 2475 | 3942 | 4.42 |
| | | G0000000201 | | | |
| 98 | AHR | CMX- | 196 | 348 | 4.41 |
| | | G0000011332 | | | |
| 99 | LIF | CMX- | 3976 | 6596 | 4.38 |
| | | G0000029949 | | | |
| 100 | PRKRA | CMX- | 8575 | 9438 | 4.38 |
| | | G0000004587 | | | |

In particular embodiments, the infertility-associated genetic region is a gene (including exons, introns, and evolutionarily conserved regions of DNA flanking either side of said gene) that impacts fertility selected from the genes shown in Table 5 below. In Table 5, HGNC (http://www.genenames.org/) reference numbers are provided when available.

Table 5 below depicts another possible gene ranking scheme for the relative infertility, subfertility, or premature decline in fertility risk associated with novel or common mutations or variants in a fertility gene. Table 5 contains the top ranked 100 fertility genes, listed in order from most to least likely for variants in that gene to affect fertility. Genetic loci are ranked according to a Celmatix Fertilome™Score, G1Version3, that reflects the likelihood a gene is involved in fertility or reproduction. This score is computed using a database of mined and curated data, containing attributes for each gene in the genome (See FIGS. 5 and 6). These attributes include: diseases and disorders related to infertility, molecular pathways, molecular interactions, gene clusters, mouse phenotypes associated with each gene, gene expression data in reproductive tissues, proteomics data in oocytes, and accrued information from scientific publications through text-mining. The Celmatix Fertilome™Score, G1Version3 differs from G1Version2 (Table 4) because it contains more fertility genes as an input for the score calculation.

TABLE 5
List of Top 100 Human Fertility Genes based on
the Fertilome ™Score, G1Version3.

| | | | | | Celmatix |
|---|---|---|---|---|---|
| | Gene | Celmatix | Entrez | HGNC | Fertilome ™ |
| Rank | Symbol | Gene ID | Gene ID | Gene ID | Score |
| 1 | C6orf221 | CMX- | 154288 | 33699 | 15 |
| | | G0000010478 | | | |
| 2 | NLRP5 | CMX- | 126206 | 21269 | 15 |
| | | G0000028192 | | | |
| 3 | TCL1A | CMX- | 8115 | 11648 | 14 |
| | | G0000021654 | | | |
| 4 | ZP3 | CMX- | 7784 | 13189 | 12.93 |
| | | G0000011947 | | | |
| 5 | FIGLA | CMX- | 344018 | 24669 | 12 |
| | | G0000003616 | | | |
| 6 | PADI6 | CMX- | 353238 | 20449 | 12 |
| | | G0000000344 | | | |
| 7 | RSPO1 | CMX- | 284654 | 21679 | 12 |
| | | G0000000687 | | | |
| 8 | EPHA1 | CMX- | 2041 | 3385 | 11.82 |
| | | G0000012650 | | | |
| 9 | DNMT1 | CMX- | 1786 | 2976 | 11.67 |
| | | G0000026880 | | | |
| 10 | ZP2 | CMX- | 7783 | 13188 | 11.67 |
| | | G0000023549 | | | |
| 11 | MOS | CMX- | 4342 | 7199 | 11.5 |
| | | G0000013392 | | | |
| 12 | FSHR | CMX- | 2492 | 3969 | 11.37 |
| | | G0000003464 | | | |
| 13 | OOEP | CMX- | 441161 | 21382 | 11 |
| | | G0000010479 | | | |
| 14 | CUL1 | CMX- | 8454 | 2551 | 10.67 |
| | | G0000012701 | | | |
| 15 | HSP90B1 | CMX- | 7184 | 12028 | 10.57 |
| | | G0000019724 | | | |
| 16 | FOXO3 | CMX- | 2309 | 3821 | 10.39 |
| | | G0000010672 | | | |
| 17 | KISS1 | CMX- | 3814 | 6341 | 10.21 |
| | | G0000002533 | | | |
| 18 | ACVR1B | CMX- | 91 | 172 | 10.14 |
| | | G0000019186 | | | |
| 19 | CGA | CMX- | 1081 | 1885 | 10.04 |
| | | G0000010560 | | | |
| 20 | INHA | CMX- | 3623 | 6065 | 10.02 |
| | | G0000004914 | | | |
| 21 | LHCGR | CMX- | 3973 | 6585 | 10.01 |
| | | G0000003462 | | | |
| 22 | DPPA3 | CMX- | 359787 | 19199 | 10 |
| | | G0000018719 | | | |
| 23 | KDM1B | CMX- | 221656 | 21577 | 10 |
| | | G0000009642 | | | |
| 24 | NOBOX | CMX- | 135935 | 22448 | 10 |
| | | G0000012690 | | | |
| 25 | NPM2 | CMX- | 10361 | 7930 | 10 |
| | | G0000013114 | | | |
| 26 | PRMT3 | CMX- | 10196 | 30163 | 10 |
| | | G0000017073 | | | |
| 27 | GJA4 | CMX- | 2701 | 4278 | 9.92 |
| | | G0000000643 | | | |
| 28 | ESR1 | CMX- | 2099 | 3467 | 9.91 |
| | | G0000011002 | | | |
| 29 | SFRP4 | CMX- | 6424 | 10778 | 9.89 |
| | | G0000011506 | | | |
| 30 | AURKA | CMX- | 6790 | 11393 | 9.84 |
| | | G0000028967 | | | |
| 31 | BRCA2 | CMX- | 675 | 1101 | 9.75 |
| | | G0000020222 | | | |
| 32 | WT1 | CMX- | 7490 | 12796 | 9.53 |
| | | G0000017126 | | | |
| 33 | CBS | CMX- | 875 | 1550 | 9.49 |
| | | G0000029408 | | | |
| 34 | CDKN1C | CMX- | 1028 | 1786 | 9.37 |
| | | G0000016717 | | | |
| 35 | IGF1 | CMX- | 3479 | 5464 | 9.35 |
| | | G0000019714 | | | |
| 36 | PLCB1 | CMX- | 23236 | 15917 | 9.33 |
| | | G0000028445 | | | |
| 37 | CEP290 | CMX- | 80184 | 29021 | 93 |
| | | G0000019604 | | | |
| 38 | MSH5 | CMX- | 4439 | 7328 | 9.29 |
| | | G0000010000 | | | |
| 39 | HAND2 | CMX- | 9464 | 4808 | 9.17 |
| | | G0000007954 | | | |
| 40 | GDF9 | CMX- | 2661 | 4224 | 9 |
| | | G0000008902 | | | |
| 41 | MAD2L1 | CMX- | 4085 | 6763 | 9 |
| | | G0000007650 | | | |
| 42 | TNFAIP6 | CMX- | 7130 | 11898 | 9 |
| | | G0000004377 | | | |
| 43 | ZAR1 | CMX- | 326340 | 20436 | 9 |
| | | G0000007128 | | | |
| 44 | FOXL2 | CMX- | 668 | 1092 | 8.88 |
| | | G0000006297 | | | |
| 45 | PCNA | CMX- | 5111 | 8729 | 8.78 |
| | | G0000028417 | | | |
| 46 | YBX2 | CMX- | 51087 | 17948 | 8.57 |
| | | G0000024578 | | | |
| 47 | BARD1 | CMX- | 580 | 952 | 8.54 |
| | | G0000004834 | | | |
| 48 | AMBP | CMX- | 259 | 453 | 8.4 |
| | | G0000014963 | | | |
| 49 | FMN2 | CMX- | 56776 | 14074 | 8.4 |
| | | G0000002910 | | | |
| 50 | NCOA2 | CMX- | 10499 | 7669 | 8.4 |
| | | G0000013477 | | | |
| 51 | TEX12 | CMX- | 56158 | 11734 | 8.4 |
| | | G0000018279 | | | |
| 52 | TACC3 | CMX- | 10460 | 11524 | 8.39 |
| | | G0000006818 | | | |
| 53 | PGR | CMX- | 5241 | 8910 | 8.37 |
| | | G0000018173 | | | |
| 54 | FANCC | CMX- | 2176 | 3584 | 8.25 |
| | | G0000014774 | | | |
| 55 | MYC | CMX- | 4609 | 7553 | 8.25 |
| | | G0000013826 | | | |
| 56 | FGF8 | CMX- | 2253 | 3686 | 8.23 |
| | | G0000016316 | | | |
| 57 | SMAD5 | CMX- | 4090 | 6771 | 8.12 |
| | | G0000008943 | | | |
| 58 | CCS | CMX- | 9973 | 1613 | 8 |
| | | G0000017793 | | | |
| 59 | MSH4 | CMX- | 4438 | 7327 | 8 |
| | | G0000001108 | | | |
| 60 | SPO11 | CMX- | 23626 | 11250 | 8 |
| | | G0000028986 | | | |
| 61 | SYCE1 | CMX- | 93426 | 28852 | 8 |
| | | G0000016602 | | | |
| 62 | SYCP1 | CMX- | 6847 | 11487 | 8 |
| | | G0000001457 | | | |
| 63 | TFAP2C | CMX- | 7022 | 11744 | 8 |
| | | G0000028982 | | | |
| 64 | WNT7A | CMX- | 7476 | 12786 | 7.96 |
| | | G0000005260 | | | |
| 65 | IL11RA | CMX- | 3590 | 5967 | 7.9 |
| | | G0000014249 | | | |
| 66 | MCM8 | CMX- | 84515 | 16147 | 7.85 |
| | | G0000028433 | | | |
| 67 | SYCP2 | CMX- | 10388 | 11490 | 7.85 |
| | | G0000029020 | | | |
| 68 | INHBA | CMX- | 3624 | 6066 | 7.83 |
| | | G0000011550 | | | |
| 69 | MGAT1 | CMX- | 4245 | 7044 | 7.83 |
| | | G0000009451 | | | |
| 70 | LHB | CMX- | 3972 | 6584 | 7.82 |
| | | G0000027859 | | | |
| 71 | CYP19A1 | CMX- | 1588 | 2594 | 7.74 |
| | | G0000022537 | | | |
| 72 | GGT1 | CMX- | 2678 | 4250 | 7.71 |
| | | G0000029874 | | | |
| 73 | TAF4B | CMX- | 6875 | 11538 | 7.68 |
| | | G0000026229 | | | |
| 74 | SMC1B | CMX- | 27127 | 11112 | 7.67 |
| | | G0000030247 | | | |
| 75 | USP9X | CMX- | 8239 | 12632 | 7.67 |
| | | G0000030612 | | | |
| 76 | PRLR | CMX- | 5618 | 9446 | 7.58 |
| | | G0000008271 | | | |
| 77 | DNMT3B | CMX- | 1789 | 2979 | 7.54 |
| | | G0000028640 | | | |
| 78 | SOD1 | CMX- | 6647 | 11179 | 7.54 |
| | | G0000029263 | | | |
| 79 | SH2B1 | CMX- | 25970 | 30417 | 7.5 |
| | | G0000023639 | | | |
| 80 | HOXA11 | CMX- | 3207 | 5101 | 7.48 |
| | | G0000011417 | | | |
| 81 | UBB | CMX- | 7314 | 12463 | 7.43 |
| | | G0000024729 | | | |
| 82 | HSF1 | CMX- | 3297 | 5224 | 7.35 |
| | | G0000013948 | | | |
| S3 | CYP17A1 | CMX- | 1586 | 2593 | 7.33 |
| | | G0000016340 | | | |
| 84 | FSHB | CMX- | 2488 | 3964 | 7.33 |
| | | G0000017113 | | | |
| 85 | SYCP3 | CMX- | 50511 | 18130 | 7.33 |
| | | G0000019706 | | | |
| 86 | NOS3 | CMX- | 4846 | 7876 | 7.31 |
| | | G0000012751 | | | |
| 87 | ZP1 | CMX- | 22917 | 13187 | 7.29 |
| | | G0000017558 | | | |
| 88 | GNRHR | CMX- | 2798 | 4421 | 7.27 |
| | | G0000007221 | | | |
| 89 | MDM2 | CMX- | 4193 | 6973 | 7.27 |
| | | G0000019503 | | | |
| 90 | BMP15 | CMX- | 9210 | 1068 | 7.25 |
| | | G0000030783 | | | |
| 91 | KDM1A | CMX- | 23028 | 29079 | 7.25 |
| | | G0000000422 | | | |
| 92 | MDK | CMX- | 4192 | 6972 | 7.21 |
| | | G0000017221 | | | |
| 93 | MSX2 | CMX- | 4488 | 7392 | 7.21 |
| | | G0000009331 | | | |
| 94 | CTNNB1 | CMX- | 1499 | 2514 | 7.2 |
| | | G0000005462 | | | |
| 95 | NR1P1 | CMX- | 8204 | 8001 | 7.2 |
| | | G0000029160 | | | |
| 96 | UBC | CMX- | 7316 | 12468 | 7.2 |
| | | G0000019992 | | | |
| 97 | FKBP4 | CMX- | 2288 | 3720 | 7.19 |
| | | G0000018615 | | | |
| 98 | MLH3 | CMX- | 27030 | 7128 | 7.14 |
| | | G0000021470 | | | |
| 99 | MSX1 | CMX- | 4487 | 7391 | 7.13 |
| | | G0000006873 | | | |
| 100 | GPC3 | CMX- | 2719 | 4451 | 7.11 |
| | | G0000031486 | | | |

In particular embodiments, the infertility-associated genetic region is a gene (including exons, introns, and evolutionarily conserved regions of DNA flanking either side of said gene) that impacts fertility selected from the genes shown in Table 6 below. In Table 5, HGNC (http://www.genenames.org/) reference numbers are provided when available.

Table 6 below depicts another possible gene ranking scheme for the relative infertility, subfertility, or premature decline in fertility risk associated with novel or common mutations or variants in a fertility gene. Table 6 contains the top ranked fertility genes based on a comparison of how often the gene appears in one of the lists above (Tables 1-5). This list represents the top 20 genetic regions with utility for diagnosing female infertility, subfertility, or premature decline in fertility. These targets were identified using a compendium of factors: 1) Carrying statistically significant genetic mutations at the coding level in a pilot study, 2) Carrying statistically significant genetic mutations at the coding level in a pilot study, 3) Carrying genetic variations in our pilot study that impact the biochemical properties of the gene, 4) Highly ranked in our Celmatix Fertilome™Score system, that reflects the likelihood a gene is involved in fertility or reproduction.

TABLE 6
List of the Top 20 Fertility Genes (arranged in alphabetical order)

| Gene | Celmatix | Entrez | HGNC |
|---|---|---|---|
| Symbol | Gene ID | Gene ID | Gene ID |
| BARD1 | CMX- | 580 | 952 |
| | G0000004834 | | |
| C6orf221 | CMX- | 154288 | 33699 |
| | G0000010478 | | |
| DNMT1 | CMX- | 1786 | 2976 |
| | G0000026880 | | |
| FMR1 | CMX- | 2332 | 3775 |
| | G0000031614 | | |
| FOXO3 | CMX- | 2309 | 3821 |
| | G0000010672 | | |
| MUC4 | CMX- | 4585 | 7514 |
| | G0000006719 | | |
| NLRP11 | CMX- | 204801 | 22945 |
| | G0000028188 | | |
| NLRP14 | CMX- | 338323 | 22939 |
| | G0000016919 | | |
| NLRP5 | CMX- | 126206 | 21269 |
| | G0000028192 | | |
| NLRP8 | CMX- | 126205 | 22940 |
| | G0000028191 | | |
| NPM2 | CMX- | 10361 | 7930 |
| | G0000013114 | | |
| PADI6 | CMX- | 353238 | 20449 |
| | G0000000344 | | |
| PMS2 | CMX- | 5395 | 9122 |
| | G0000011251 | | |
| SCARB1 | CMX- | 949 | 1664 |
| | G0000019991 | | |
| SPIN1 | CMX- | 10927 | 11243 |
| | G0000014689 | | |
| TACC3 | CMX- | 10460 | 11524 |
| | G0000006818 | | |
| ZP1 | CMX- | 22917 | 13187 |
| | G0000017558 | | |
| ZP2 | CMX- | 7783 | 13188 |
| | G0000023549 | | |
| ZP3 | CMX- | 7784 | 13189 |
| | G0000011947 | | |
| ZP4 | CMX- | 57829 | 15770 |
| | G0000002903 | | |

In particular embodiments, the infertility-associated genetic region is a gene (including exons, introns, and evolutionarily conserved regions of DNA flanking either side of said gene) that impacts fertility selected from the genes shown in Table 7 below. In Table 7, HGNC (http://www.genenames.org/) reference numbers are provided when available.

Table 7 below depicts all of the biologically and/or statistically significant variants detected in the genes depicted in Table 6 in a genetic study of female infertility. Genetic variants considered to be biologically significant include mutations that result in a change: 1) to a different amino acid predicted to alter the folding and/or structure of the encoded protein, 2) to a different amino acid occurring at a highly evolutionarily conserved site, 3) that introduces a premature stop termination signal, 4) that causes a stop termination signal to be lost, 5) that introduces a new start codon, 6) that causes a start codon to be lost, 7) that disrupts a splicing signal, 8) that alters the reading frame or 9) that alters the dosage of encoded protein or RNA. All genetic variants detected from resequencing exclude sites at the single nucleotide level where the variant allele is detected in only one chromosome (singletons) and sites sequenced in only one individual. Structural variants impacting biological function are also reported. Using these criteria applied to targeted re-sequencing data from a study of infertile females, we detected 490 variants, of which 379 are listed in Table 7.

For the statistically significant variant level analysis, a series of logistic regression models are fit, where the outcome variable is the binary indicator of variant status for a given location, and the independent variables are group (infertile vs. control) and principal component-derived ethnicity (continuous). The p-value and odds ratio for group are used for statistical inference. The model is fit once for each location. P-values<0.001 are considered statistically significant. We performed a SNP association study by targeted re-sequencing and identified a total of 147 SNPs significantly associated with female infertility (of which 52 are reported in Table 7). Each variant was classified as novel or known. Novel sites are excluded from the p-value computation. For known variants, we apply a series of logistic regression models where the outcome variable is the binary indicator of variant status for a given location, and the independent variables are group (infertile vs. control) and principal component-derived ethnicity (continuous). The p-value and odds ratio for group are used for statistical inference. P-values less than 0.001 were considered significant. Position refers to NCBI Build 37. Alleles are reported on the forward strand. Ref=Reference allele, Alt=Variant allele.

TABLE 7
List of Biologically and Statistically Significant Genetic Variants Most
Useful for Predicting Infertility Risk in Humans (arranged in alphabetical order by
gene name)

| Gene | Celmatix | Celmatix | | | | | P- |
|---|---|---|---|---|---|---|---|
| Symbol | Gene ID | Variant ID | Location | Ref | Alt | Impact | value |
| APOA1 | CMX- | CMX- | chr11: 112553969-126265772 | NA | CNV | APOA1 (3 | NA |
| | G0000018327 | V1388879 | | | gain | exons) | |
| ASCL2 | CMX- | CMX- | chr11: 2234334-2298706 | NA | CNV | ASCL2 (1 | NA |
| | G0000016707 | V1067111 | | | gain | exon) | |
| BARD1 | CMX- | CMX- | chr2: 215674224 | G | A | Drastic | NA |
| | G0000004834 | V9083698 | | | | nonsynonymous | |
| BARD1 | CMX- | CMX- | chr2: 215595645 | C | T | Start codon | NA |
| | G0000004834 | V9083699 | | | | lost | |
| BARD1 | CMX- | CMX- | chr2: 215674323 | C | G | Start codon | NA |
| | G0000004834 | V9083700 | | | | gained | |
| BARD1 | CMX- | CMX- | chr2: 215645502 | GTGGTG | G | Codon deletion | NA |
| | G0000004834 | SV00001 | | AAGAAC | | | |
| | | | | ATTCAG | | | |
| | | | | GCAA | | | |
| BARD1 | CMX- | CMX- | chr2: 215742204 | G | T | NA | 6.77E−05 |
| | G0000004834 | V9084177 | | | | | |
| BMP15 | CMX- | CMX- | chrX: 50639969-50981841 | NA | CNV | BMP15 (2 | NA |
| | G0000030783 | V1250077 | | | gain | exons) | |
| BMP6 | CMX- | CMX- | chr6: 7726514-7727614 | NA | CNV | BMP6 (1 | NA |
| | G0000009564 | V1247770 | | | loss | exon) | |
| BMP6 | CMX- | CMX- | chr6: 7724859-7728905 | NA | CNV | BMP6 (1 | NA |
| | G0000009564 | V1166409 | | | loss | exon) | |
| C6orf221 | CMX- | CMX- | chr6: 74073531 | C | G | Drastic | NA |
| | G0000010478 | V9083706 | | | | nonsynonymous | |
| CASP8 | CMX- | CMX- | chr2: 201851129-203110758 | NA | CNV | CASP8 (2 | NA |
| | G0000004721 | V1843349 | | | loss | exons) | |
| CSF1, UBL4B | CMX- | CMX- | chr1: 110441465-110831379 | NA | CNV | CSF1 (4 | NA |
| | G0000001374, | V1667025 | | | loss | exons), | |
| | CMX- | | | | | UBL4B (1 | |
| | G0000001378 | | | | | exon) | |
| CSF2 | CMX- | CMX- | chr5: 128320218-131440732 | NA | CNV | CSF2 (4 | NA |
| | G0000008885 | V1456214 | | | loss | exons) | |
| CYP11B1 | CMX- | CMX- | chr8: 143951813-143958440 | NA | CNV | CYP11B1 (4 | NA |
| | G0000013888 | V1957973 | | | gain | exons) | |
| CYP11B1 | CMX- | CMX- | chr8: 143953403-143991713 | NA | CNV | CYP11B1 (4 | NA |
| | G0000013888 | V1609269 | | | gain | exons) | |
| DCTPP1, | CMX- | CMX- | chr16: 30347689-31632796 | NA | CNV | DCTPP1 (1 | NA |
| SEPHS2, | G0000023705, | V1070550 | | | gain | exon), | |
| TGFB1I1, | CMX- | | | | | SEPHS2 (1 | |
| VKORC1 | G0000023707, | | | | | exon), | |
| | CMX- | | | | | TGFB1I1 (3 | |
| | G0000023757, | | | | | exons), | |
| | CMX- | | | | | VKORC1 (1 | |
| | G0000023741 | | | | | exon) | |
| DNMT1 | CMX- | CMX- | chr19: 10291181 | T | C | Drastic | NA |
| | G0000026880 | V9083720 | | | | nonsynonymous | |
| ECHS1 | CMX- | CMX- | chr10: 135087081-135243330 | NA | CNV | ECHS1 (8 | NA |
| | G0000016594 | V1101514 | | | gain | exons) | |
| ECHS1 | CMX- | CMX- | chr10: 135088839-135243616 | NA | CNV | ECHS1 (8 | NA |
| | G0000016594 | V1131837 | | | loss | exons) | |
| ECHS1 | CMX- | CMX- | chr10: 135087962-135243616 | NA | CNV | ECHS1 (8 | NA |
| | G0000016594 | V1335364 | | | gain | exons) | |
| EFNA4 | CMX- | CMX- | chr1: 154354576-155066744 | NA | CNV | EFNA4 (4 | NA |
| | G0000001896 | V1267541 | | | loss | exons) | |
| EFNB3 | CMX- | CMX- | chr17: 7135639-7702377 | NA | CNV | EFNB3 (5 | NA |
| | G0000024616 | V1295730 | | | gain | exons) | |
| EIF3CL | CMX- | CMX- | chr16: 28197032-28410526 | NA | CNV | EIF3CL (13 | NA |
| | G0000023621 | V1992389 | | | loss | exons) | |
| EPHA5 | CMX- | CMX- | chr4: 66114884-66870165 | NA | CNV | EPHA5 (17 | NA |
| | G0000007213 | V1585842 | | | loss | exons) | |
| EPHA7 | CMX- | CMX- | chr6: 94015504-95364976 | NA | CNV | EPHA7 (3 | NA |
| | G0000010603 | V1939194 | | | loss | exons) | |
| EPHA8 | CMX- | CMX- | chr1: 22906197-22914076 | NA | CNV | EPHA8 (1 | NA |
| | G0000000415 | V1493926 | | | loss | exon) | |
| EPHA8 | CMX- | CMX- | chr1: 22905731-22915711 | NA | CNV | EPHA8 (2 | NA |
| | G0000000415 | V1680494 | | | loss | exons) | |
| EPHA8 | CMX- | CMX- | chr1: 22904786-22915711 | NA | CNV | EPHA8 (2 | NA |
| | G0000000415 | V1333389 | | | loss | exons) | |
| EPHA8 | CMX- | CMX- | chr1: 22906271-22915711 | NA | CNV | EPHA8 (2 | NA |
| | G0000000415 | V1750787 | | | loss | exons) | |
| EPHA8 | CMX- | CMX- | chr1: 22906197-22915047 | NA | CNV | EPHA8 (1 | NA |
| | G0000000415 | V1102470 | | | loss | exon) | |
| EPHA8 | CMX- | CMX- | chr1: 22905731-22915352 | NA | CNV | EPHA8 (1 | NA |
| | G0000000415 | V1356293 | | | loss | exon) | |
| EPHA8 | CMX- | CMX- | chr1: 22905731-22913963 | NA | CNV | EPHA8 (1 | NA |
| | G0000000415 | V1845595 | | | loss | exon) | |
| EPHA8 | CMX- | CMX- | chr1: 22906526-22913011 | NA | CNV | EPHA8 (1 | NA |
| | G0000000415 | V1973671 | | | loss | exon) | |
| EPHA8 | CMX- | CMX- | chr1: 22905731-22916983 | NA | CNV | EPHA8 (2 | NA |
| | G0000000415 | V1086453 | | | loss | exons) | |
| EPHA8 | CMX- | CMX- | chr1: 22904856-22913700 | NA | CNV | EPHA8 (1 | NA |
| | G0000000415 | V1138079 | | | loss | exon) | |
| EPHA8 | CMX- | CMX- | chr1: 22904786-22914210 | NA | CNV | EPHA8 (1 | NA |
| | G0000000415 | V1957426 | | | loss | exon) | |
| EPHA8 | CMX- | CMX- | chr1: 22906197-22915352 | NA | CNV | EPHA8 (1 | NA |
| | G0000000415 | V1635641 | | | loss | exon) | |
| EPHA8 | CMX- | CMX- | chr1: 22905731-22914256 | NA | CNV | EPHA8 (1 | NA |
| | G0000000415 | V1387198 | | | loss | exon) | |
| EPHA8 | CMX- | CMX- | chr1: 22906271-22913750 | NA | CNV | EPHA8 (1 | NA |
| | G0000000415 | V1481340 | | | loss | exon) | |
| EPHA8 | CMX- | CMX- | chr1: 22904856-22913963 | NA | CNV | EPHA8 (1 | NA |
| | G0000000415 | V1077862 | | | loss | exon) | |
| EPHA8 | CMX- | CMX- | chr1: 22904064-22914256 | NA | CNV | EPHA8 (1 | NA |
| | G0000000415 | V1288029 | | | loss | exon) | |
| EPHA8 | CMX- | CMX- | chr1: 22906395-22913750 | NA | CNV | EPHA8 (1 | NA |
| | G0000000415 | V1098423 | | | loss | exon) | |
| EPHA8 | CMX- | CMX- | chr1: 22906271-22914210 | NA | CNV | EPHA8 (1 | NA |
| | G0000000415 | V1825294 | | | loss | exon) | |
| EPHA8 | CMX- | CMX- | chr1: 22906271-22915161 | NA | CNV | EPHA8 (1 | NA |
| | G0000000415 | V1672255 | | | loss | exon) | |
| EPHA8 | CMX- | CMX- | chr1: 22906271-22914076 | NA | CNV | EPHA8 (1 | NA |
| | G0000000415 | V1740010 | | | loss | exon) | |
| EPHA8 | CMX- | CMX- | chr1: 22904856-22915352 | NA | CNV | EPHA8 (1 | NA |
| | G0000000415 | V1757241 | | | loss | exon) | |
| EPHA8 | CMX- | CMX- | chr1: 22906322-22914695 | NA | CNV | EPHA8 (1 | NA |
| | G0000000415 | V1080982 | | | loss | exon) | |
| EPHA8 | CMX- | CMX- | chr1: 22905731-22913502 | NA | CNV | EPHA8 (1 | NA |
| | G0000000415 | V1506728 | | | loss | exon) | |
| FGF8 | CMX- | CMX- | chr10: 103524444-103533748 | NA | CNV | FGF8 (2 | NA |
| | G0000016316 | V1202186 | | | gain | exons) | |
| FGF8 | CMX- | CMX- | chr10: 103524714-103532892 | NA | CNV | FGF8 (2 | NA |
| | G0000016316 | V1242750 | | | gain | exons) | |
| FGF8 | CMX- | CMX- | chr10: 103520069-103531134 | NA | CNV | FGF8 (1 exon) | NA |
| | G0000016316 | V1059642 | | | gain | | |
| FGF8 | CMX- | CMX- | chr10: 103525082-103536399 | NA | CNV | FGF8 (6 | NA |
| | G0000016316 | V1478224 | | | gain | exons) | |
| FMR1 | CMX- | CMX- | chrX: 147010263 | A | C | Drastic | NA |
| | G0000031614 | V9083727 | | | | nonsynonymous | |
| FMR1 | CMX- | CMX- | chrX: 147014960 | C | T | Start codon | NA |
| | G0000031614 | V9083728 | | | | gained | |
| FMR1 | CMX- | CMX- | chrX: 146126483 | G | A | NA | 0.000198744 |
| | G0000031614 | V9084252 | | | | | |
| FMR1 | CMX- | CMX- | chrX: 146153970 | C | T | NA | 1.92E−05 |
| | G0000031614 | V9084253 | | | | | |
| FMR1 | CMX- | CMX- | chrX: 146195865 | A | G | NA | 0.000371198 |
| | G0000031614 | V9084254 | | | | | |
| FMR1 | CMX- | CMX- | chrX: 146221514 | C | T | NA | 0.000292157 |
| | G0000031614 | V9084255 | | | | | |
| FMR1 | CMX- | CMX- | chrX: 146247740 | T | A | NA | 0.0001997 |
| | G0000031614 | V9084256 | | | | | |
| FMR1 | CMX- | CMX- | chrX: 146255213 | G | A | NA | 0.000185975 |
| | G0000031614 | V9084257 | | | | | |
| FMR1 | CMX- | CMX- | chrX: 146406319 | A | G | NA | 0.000262855 |
| | G0000031614 | V9084258 | | | | | |
| FMR1 | CMX- | CMX- | chrX: 146994916 | A | G | NA | 0.000816693 |
| | G0000031614 | V9084259 | | | | | |
| FMR1 | CMX- | CMX- | chrX: 147002992 | T | G | NA | 0.000810806 |
| | G0000031614 | V9084260 | | | | | |
| FMR1 | CMX- | CMX- | chrX: 147003339 | A | G | NA | 0.000810806 |
| | G0000031614 | V9084261 | | | | | |
| FMR1 | CMX- | CMX- | chrX: 147003794 | T | C | NA | 0.000810806 |
| | G0000031614 | V9084262 | | | | | |
| FMR1 | CMX- | CMX- | chrX: 147024558 | A | T | NA | 0.000641561 |
| | G0000031614 | V9084263 | | | | | |
| FMR1 | CMX- | CMX- | chrX: 147372528 | G | C | NA | 0.000633948 |
| | G0000031614 | V9084264 | | | | | |
| FMR1 | CMX- | CMX- | chrX: 147397806 | A | G | NA | 0.000813685 |
| | G0000031614 | V9084265 | | | | | |
| FMR1 | CMX- | CMX- | chrX: 147437683 | A | G | NA | 0.000784981 |
| | G0000031614 | V9084266 | | | | | |
| FMR1 | CMX- | CMX- | chrX: 147449673 | T | C | NA | 0.000401568 |
| | G0000031614 | V9084267 | | | | | |
| FMR1 | CMX- | CMX- | chrX: 147454832 | G | A | NA | 0.000965078 |
| | G0000031614 | V9084268 | | | | | |
| FMR1 | CMX- | CMX- | chrX: 147478274 | G | T | NA | 0.000646517 |
| | G0000031614 | V9084269 | | | | | |
| FMR1 | CMX- | CMX- | chrX: 147479861 | A | C | NA | 0.000646517 |
| | G0000031614 | V9084270 | | | | | |
| FMR1 | CMX- | CMX- | chrX: 147480274 | A | G | NA | 0.000646517 |
| | G0000031614 | V9084271 | | | | | |
| FMR1 | CMX- | CMX- | chrX: 147481891 | T | C | NA | 0.000646517 |
| | G0000031614 | V9084272 | | | | | |
| FMR1 | CMX- | CMX- | chrX: 147482603 | A | G | NA | 0.000564877 |
| | G0000031614 | V9084273 | | | | | |
| FMR1 | CMX- | CMX- | chrX: 147482630 | A | G | NA | 0.000458631 |
| | G0000031614 | V9084274 | | | | | |
| FOXO3 | CMX- | CMX- | chr6: 108856108 | C | T | NA | 0.000232121 |
| | G0000010672 | V9084196 | | | | | |
| FOXO3 | CMX- | CMX- | chr6: 109149693 | G | C | NA | 0.000344433 |
| | G0000010672 | V9084197 | | | | | |
| FOXO3 | CMX- | CMX- | chr6: 108853361 | T | A | NA | 0.000176018 |
| | G0000010672 | V9084195 | | | | | |
| FOXO3 | CMX- | CMX- | chr6: 109155789 | G | T | NA | 0.000641107 |
| | G0000010672 | V9084198 | | | | | |
| FOXO3 | CMX- | CMX- | chr6: 108985148-108989762 | NA | CNV | FOXO3 (1 | NA |
| | G0000010672 | V1295244 | | | gain | exon) | |
| FOXO3 | CMX- | CMX- | chr6: 108985507-108989056 | NA | CNV | FOXO3 (1 | NA |
| | G0000010672 | V1963522 | | | gain | exon) | |
| FOXO3 | CMX- | CMX- | chr6: 108984930-108989762 | NA | CNV | FOXO3 (1 | NA |
| | G0000010672 | V1616823 | | | gain | exon) | |
| FOXP3 | CMX- | CMX- | chrX: 48890221-49257528 | NA | CNV | FOXP3 (9 | NA |
| | G0000030750 | V1008919 | | | gain | exons) | |
| GDF1 | CMX- | CMX- | chr19: 18872185-19535389 | NA | CNV | GDF1 (2 | NA |
| | G0000027183 | V1625432 | | | gain | exons) | |
| GJA4, GJB3, | CMX- | CMX- | chr1: 35000925-37866010 | NA | CNV | GJA4 (1 | NA |
| GJB4 | G0000000643, | V1706868 | | | gain | exon), GJB3 (1 | |
| | CMX- | | | | | exon), GJB4 (1 | |
| | G0000000642, | | | | | exon) | |
| | CMX- | | | | | | |
| | G0000000641 | | | | | | |
| GJD3 | CMX- | CMX- | chr17: 37952541-38532715 | NA | CNV | GJD3 (1 exon) | NA |
| | G0000025169 | V1132225 | | | gain | | |
| GPC3 | CMX- | CMX- | chrX: 132613906-132779666 | NA | CNV | GPC3 (1 exon) | NA |
| | G0000031486 | V1515961 | | | gain | | |
| IGF2 | CMX- | CMX- | chr11: 2127129-2173473 | NA | CNV | IGF2 (3 exons) | NA |
| | G0000016702 | V1454080 | | | gain | | |
| IGF2 | CMX- | CMX- | chr11: 2110901-2173938 | NA | CNV | IGF2 (3 exons) | NA |
| | G0000016702 | V1542559 | | | gain | | |
| IGFBPL1 | CMX- | CMX- | chr9: 35776310-38419649 | NA | CNV | IGFBL1 (3 | NA |
| | G0000014341 | V1435664 | | | loss | exons) | |
| ISG15 | CMX- | CMX- | chr1: 940142-1016233 | NA | CNV | ISG15 (2 | NA |
| | G0000000029 | V1111642 | | | gain | exons) | |
| ISG15 | CMX- | CMX- | chr1: 834638-1271900 | NA | CNV | ISG15 (2 | NA |
| | G0000000029 | V1884847 | | | gain | exons) | |
| KISS1 | CMX- | CMX- | chr1: 202729101-205013246 | NA | CNV | KISS1 (2 | NA |
| | G0000002533 | V1823995 | | | gain | exons) | |
| KISS1R | CMX- | CMX- | chr19: 867728-945645 | NA | CNV | KISS1R (2 | NA |
| | G0000026560 | V1469394 | | | gain | exons) | |
| KISS1R | CMX- | CMX- | chr19: 867728-1126103 | NA | CNV | KISS1R (2 | NA |
| | G0000026560 | V1974120 | | | gain | exons) | |
| KISS1R | CMX- | CMX- | chr19: 868013-1085518 | NA | CNV | KISS1R (2 | NA |
| | G0000026560 | V1813360 | | | gain | exons) | |
| KISS1R | CMX- | CMX- | chr19: 866589-1232099 | NA | CNV | KISS1R (2 | NA |
| | G0000026560 | V1883755 | | | gain | exons) | |
| LOXL4 | CMX- | CMX- | chr10: 100013106-100022354 | NA | CNV | LOXL4 (9 | NA |
| | G0000016263 | V1039367 | | | loss | exons) | |
| LOXL4 | CMX- | CMX- | chr10: 100013359-100023161 | NA | CNV | LOXL4 (10 | NA |
| | G0000016263 | V1620875 | | | loss | exons) | |
| LOXL4 | CMX- | CMX- | chr10: 100014360-100020546 | NA | CNV | LOXL4 (6 | NA |
| | G0000016263 | V1806767 | | | loss | exons) | |
| LOXL4 | CMX- | CMX- | chr10: 100014176-100022354 | NA | CNV | LOXL4 (8 | NA |
| | G0000016263 | V1954806 | | | loss | exons) | |
| LOXL4 | CMX- | CMX- | chr10: 100015459-100023313 | NA | CNV | LOXL4 (9 | NA |
| | G0000016263 | V1107311 | | | loss | exons) | |
| LOXL4 | CMX- | CMX- | chr10: 100015459-100023369 | NA | CNV | LOXL4 (9 | NA |
| | G0000016263 | V1373344 | | | loss | exons) | |
| LOXL4 | CMX- | CMX- | chr10: 100015459-100023161 | NA | CNV | LOXL4 (9 | NA |
| | G0000016263 | V1073572 | | | loss | exons) | |
| LOXL4 | CMX- | CMX- | chr10: 100014551-100023161 | NA | CNV | LOXL4 (9 | NA |
| | G0000016263 | V1348325 | | | loss | exons) | |
| LOXL4 | CMX- | CMX- | chr10: 100011910-100023369 | NA | CNV | LOXL4 (11 | NA |
| | G0000016263 | V1321127 | | | loss | exons) | |
| LOXL4 | CMX- | CMX- | chr10: 100013876-103528663 | NA | CNV | LOXL4 (9 | NA |
| | G0000016263 | V1323761 | | | loss | exons) | |
| LOXL4 | CMX- | CMX- | chr10: 100014176-100023161 | NA | CNV | LOXL4 (9 | NA |
| | G0000016263 | V1275468 | | | loss | exons) | |
| MAP3K2 | CMX- | CMX- | chr2: 128093608-128138545 | NA | CNV | MAP3K2 (3 | NA |
| | G0000004205 | V1566424 | | | gain | exons) | |
| MAP3K2 | CMX- | CMX- | chr2: 128098216-128117112 | NA | CNV | MAP3K2 (1 | NA |
| | G0000004205 | V1811137 | | | gain | exon) | |
| MAP3K2 | CMX- | CMX- | chr2: 127520276-128116794 | NA | CNV | MAP3K2 (16 | NA |
| | G0000004205 | V1696049 | | | gain | exons) | |
| MUC4 | CMX- | CMX- | chr3: 195505739 | C | T | Drastic | NA |
| | G0000006719 | V9083756 | | | | nonsynonymous | |
| MUC4 | CMX- | CMX- | chr3: 195505960 | G | C | Drastic | NA |
| | G0000006719 | V9083757 | | | | nonsynonymous | |
| MUC4 | CMX- | CMX- | chr3: 195506089 | G | A | Drastic | NA |
| | G0000006719 | V9083758 | | | | nonsynonymous | |
| MUC4 | CMX- | CMX- | chr3: 195506099 | T | C | Drastic | NA |
| | G0000006719 | V9083759 | | | | nonsynonymous | |
| MUC4 | CMX- | CMX- | chr3: 195505883 | T | C | Drastic | NA |
| | G0000006719 | V9083760 | | | | nonsynonymous | |
| MUC4 | CMX- | CMX- | chr3: 195501149 | C | T | Drastic | NA |
| | G0000006719 | V9083761 | | | | nonsynonymous | |
| MUC4 | CMX- | CMX- | chr3: 195506156 | G | C | Drastic | NA |
| | G0000006719 | V9083762 | | | | nonsynonymous | |
| MUC4 | CMX- | CMX- | chr3: 195505897 | G | A | Drastic | NA |
| | G0000006719 | V9083763 | | | | nonsynonymous | |
| MUC4 | CMX- | CMX- | chr3: 195506146 | A | G | Drastic | NA |
| | G0000006719 | V9083764 | | | | nonsynonymous | |
| MUC4 | CMX- | CMX- | chr3: 195506149 | C | T | Drastic | NA |
| | G0000006719 | V9083765 | | | | nonsynonymous | |
| MUC4 | CMX- | CMX- | chr3: 195506281 | A | G | Drastic | NA |
| | G0000006719 | V9083766 | | | | nonsynonymous | |
| MUC4 | CMX- | CMX- | chr3: 195506291 | C | T | Drastic | NA |
| | G0000006719 | V9083767 | | | | nonsynonymous | |
| MUC4 | CMX- | CMX- | chr3: 195506302 | G | T | Drastic | NA |
| | G0000006719 | V9083768 | | | | nonsynonymous | |
| MUC4 | CMX- | CMX- | chr3: 195506245 | C | A | Drastic | NA |
| | G0000006719 | V9083769 | | | | nonsynonymous | |
| MUC4 | CMX- | CMX- | chr3: 195495916 | G | C | Drastic | NA |
| | G0000006719 | V9083770 | | | | nonsynonymous | |
| MUC4 | CMX- | CMX- | chr3: 195506318 | C | G | Drastic | NA |
| | G0000006719 | V9083771 | | | | nonsynonymous | |
| MUC4 | CMX- | CMX- | chr3: 195506323 | G | C | Drastic | NA |
| | G0000006719 | V9083772 | | | | nonsynonymous | |
| MUC4 | CMX- | CMX- | chr3: 195506339 | T | G | Drastic | NA |
| | G0000006719 | V9083773 | | | | nonsynonymous | |
| MUC4 | CMX- | CMX- | chr3: 195506350 | G | T | Drastic | NA |
| | G0000006719 | V9083774 | | | | nonsynonymous | |
| MUC4 | CMX- | CMX- | chr3: 195506364 | G | C | Drastic | NA |
| | G0000006719 | V9083775 | | | | nonsynonymous | |
| MUC4 | CMX- | CMX- | chr3: 195506185 | G | A | Drastic | NA |
| | G0000006719 | V9083776 | | | | nonsynonymous | |
| MUC4 | CMX- | CMX- | chr3: 195506195 | C | T | Drastic | NA |
| | G0000006719 | V9083777 | | | | nonsynonymous | |
| MUC4 | CMX- | CMX- | chr3: 195506398 | G | T | Drastic | NA |
| | G0000006719 | V9083778 | | | | nonsynonymous | |
| MUC4 | CMX- | CMX- | chr3: 195506410 | G | A | Drastic | NA |
| | G0000006719 | V9083779 | | | | nonsynonymous | |
| MUC4 | CMX- | CMX- | chr3: 195506411 | C | T | Drastic | NA |
| | G0000006719 | V9083780 | | | | nonsynonymous | |
| MUC4 | CMX- | CMX- | chr3: 195506446 | G | T | Drastic | NA |
| | G0000006719 | V9083781 | | | | nonsynonymous | |
| MUC4 | CMX- | CMX- | chr3: 195506460 | G | C | Drastic | NA |
| | G0000006719 | V9083782 | | | | nonsynonymous | |
| MUC4 | CMX- | CMX- | chr3: 195506005 | A | C | Drastic | NA |
| | G0000006719 | V9083783 | | | | nonsynonymous | |
| MUC4 | CMX- | CMX- | chr3: 195506521 | G | A | Drastic | NA |
| | G0000006719 | V9083784 | | | | nonsynonymous | |
| MUC4 | CMX- | CMX- | chr3: 195506533 | C | A | Drastic | NA |
| | G0000006719 | V9083785 | | | | nonsynonymous | |
| MUC4 | CMX- | CMX- | chr3: 195506542 | G | T | Drastic | NA |
| | G0000006719 | V9083786 | | | | nonsynonymous | |
| MUC4 | CMX- | CMX- | chr3: 195505788 | G | C | Drastic | NA |
| | G0000006719 | V9083787 | | | | nonsynonymous | |
| MUC4 | CMX- | CMX- | chr3: 195506558 | G | C | Drastic | NA |
| | G0000006719 | V9083788 | | | | nonsynonymous | |
| MUC4 | CMX- | CMX- | chr3: 195506590 | G | A | Drastic | NA |
| | G0000006719 | V9083789 | | | | nonsynonymous | |
| MUC4 | CMX- | CMX- | chr3: 195506597 | G | A | Drastic | NA |
| | G0000006719 | V9083790 | | | | nonsynonymous | |
| MUC4 | CMX- | CMX- | chr3: 195505906 | G | A | Drastic | NA |
| | G0000006719 | V9083791 | | | | nonsynonymous | |
| MUC4 | CMX- | CMX- | chr3: 195506626 | G | A | Drastic | NA |
| | G0000006719 | V9083792 | | | | nonsynonymous | |
| MUC4 | CMX- | CMX- | chr3: 195506627 | T | G | Drastic | NA |
| | G0000006719 | V9083793 | | | | nonsynonymous | |
| MUC4 | CMX- | CMX- | chr3: 195506740 | G | C | Drastic | NA |
| | G0000006719 | V9083794 | | | | nonsynonymous | |
| MUC4 | CMX- | CMX- | chr3: 195506746 | G | A | Drastic | NA |
| | G0000006719 | V9083795 | | | | nonsynonymous | |
| MUC4 | CMX- | CMX- | chr3: 195506494 | G | T | Drastic | NA |
| | G0000006719 | V9083796 | | | | nonsynonymous | |
| MUC4 | CMX- | CMX- | chr3: 195506750 | G | C | Drastic | NA |
| | G0000006719 | V9083797 | | | | nonsynonymous | |
| MUC4 | CMX- | CMX- | chr3: 195506752 | C | T | Drastic | NA |
| | G0000006719 | V9083798 | | | | nonsynonymous | |
| MUC4 | CMX- | CMX- | chr3: 195506753 | G | C | Drastic | NA |
| | G0000006719 | V9083799 | | | | nonsynonymous | |
| MUC4 | CMX- | CMX- | chr3: 195506809 | G | T | Drastic | NA |
| | G0000006719 | V9083800 | | | | nonsynonymous | |
| MUC4 | CMX- | CMX- | chr3: 195506914 | G | A | Drastic | NA |
| | G0000006719 | V9083801 | | | | nonsynonymous | |
| MUC4 | CMX- | CMX- | chr3: 195506917 | A | C | Drastic | NA |
| | G0000006719 | V9083802 | | | | nonsynonymous | |
| MUC4 | CMX- | CMX- | chr3: 195506933 | G | A | Drastic | NA |
| | G0000006719 | V9083803 | | | | nonsynonymous | |
| MUC4 | CMX- | CMX- | chr3: 195506940 | G | C | Drastic | NA |
| | G0000006719 | V9083804 | | | | nonsynonymous | |
| MUC4 | CMX- | CMX- | chr3: 195506953 | G | A | Drastic | NA |
| | G0000006719 | V9083805 | | | | nonsynonymous | |
| MUC4 | CMX- | CMX- | chr3: 195506965 | T | C | Drastic | NA |
| | G0000006719 | V9083806 | | | | nonsynonymous | |
| MUC4 | CMX- | CMX- | chr3: 195506966 | C | T | Drastic | NA |
| | G0000006719 | V9083807 | | | | nonsynonymous | |
| MUC4 | CMX- | CMX- | chr3: 195506975 | G | C | Drastic | NA |
| | G0000006719 | V9083808 | | | | nonsynonymous | |
| MUC4 | CMX- | CMX- | chr3: 195506747 | C | T | Drastic | NA |
| | G0000006719 | V9083809 | | | | nonsynonymous | |
| MUC4 | CMX- | CMX- | chr3: 195506986 | G | A | Drastic | NA |
| | G0000006719 | V9083810 | | | | nonsynonymous | |
| MUC4 | CMX- | CMX- | chr3: 195506987 | T | C | Drastic | NA |
| | G0000006719 | V9083811 | | | | nonsynonymous | |
| MUC4 | CMX- | CMX- | chr3: 195506990 | C | G | Drastic | NA |
| | G0000006719 | V9083812 | | | | nonsynonymous | |
| MUC4 | CMX- | CMX- | chr3: 195507010 | A | G | Drastic | NA |
| | G0000006719 | V9083813 | | | | nonsynonymous | |
| MUC4 | CMX- | CMX- | chr3: 195507059 | T | C | Drastic | NA |
| | G0000006719 | V9083814 | | | | nonsynonymous | |
| MUC4 | CMX- | CMX- | chr3: 195507062 | C | T | Drastic | NA |
| | G0000006719 | V9083815 | | | | nonsynonymous | |
| MUC4 | CMX- | CMX- | chr3: 195506378 | C | A | Drastic | NA |
| | G0000006719 | V9083816 | | | | nonsynonymous | |
| MUC4 | CMX- | CMX- | chr3: 195507083 | T | C | Drastic | NA |
| | G0000006719 | V9083817 | | | | nonsynonymous | |
| MUC4 | CMX- | CMX- | chr3: 195507086 | C | G | Drastic | NA |
| | G0000006719 | V9083818 | | | | nonsynonymous | |
| MUC4 | CMX- | CMX- | chr3: 195507107 | C | T | Drastic | NA |
| | G0000006719 | V9083819 | | | | nonsynonymous | |
| MUC4 | CMX- | CMX- | chr3: 195507166 | A | G | Drastic | NA |
| | G0000006719 | V9083820 | | | | nonsynonymous | |
| MUC4 | CMX- | CMX- | chr3: 195507203 | T | G | Drastic | NA |
| | G0000006719 | V9083821 | | | | nonsynonymous | |
| MUC4 | CMX- | CMX- | chr3: 195507226 | A | G | Drastic | NA |
| | G0000006719 | V9083822 | | | | nonsynonymous | |
| MUC4 | CMX- | CMX- | chr3: 195507228 | G | C | Drastic | NA |
| | G0000006719 | V9083823 | | | | nonsynonymous | |
| MUC4 | CMX- | CMX- | chr3: 195507236 | T | C | Drastic | NA |
| | G0000006719 | V9083824 | | | | nonsynonymous | |
| MUC4 | CMX- | CMX- | chr3: 195507242 | C | A | Drastic | NA |
| | G0000006719 | V9083825 | | | | nonsynonymous | |
| MUC4 | CMX- | CMX- | chr3: 195507251 | G | T | Drastic | NA |
| | G0000006719 | V9083826 | | | | nonsynonymous | |
| MUC4 | CMX- | CMX- | chr3: 195507262 | T | G | Drastic | NA |
| | G0000006719 | V9083827 | | | | nonsynonymous | |
| MUC4 | CMX- | CMX- | chr3: 195507316 | G | A | Drastic | NA |
| | G0000006719 | V9083828 | | | | nonsynonymous | |
| MUC4 | CMX- | CMX- | chr3: 195507323 | T | C | Drastic | NA |
| | G0000006719 | V9083829 | | | | nonsynonymous | |
| MUC4 | CMX- | CMX- | chr3: 195507324 | G | C | Drastic | NA |
| | G0000006719 | V9083830 | | | | nonsynonymous | |
| MUC4 | CMX- | CMX- | chr3: 195507365 | G | A | Drastic | NA |
| | G0000006719 | V9083831 | | | | nonsynonymous | |
| MUC4 | CMX- | CMX- | chr3: 195507379 | G | C | Drastic | NA |
| | G0000006719 | V9083832 | | | | nonsynonymous | |
| MUC4 | CMX- | CMX- | chr3: 195507385 | G | A | Drastic | NA |
| | G0000006719 | V9083833 | | | | nonsynonymous | |
| MUC4 | CMX- | CMX- | chr3: 195507397 | T | C | Drastic | NA |
| | G0000006719 | V9083834 | | | | nonsynonymous | |
| MUC4 | CMX- | CMX- | chr3: 195507398 | C | T | Drastic | NA |
| | G0000006719 | V9083835 | | | | nonsynonymous | |
| MUC4 | CMX- | CMX- | chr3: 195507406 | G | A | Drastic | NA |
| | G0000006719 | V9083836 | | | | nonsynonymous | |
| MUC4 | CMX- | CMX- | chr3: 195507412 | C | G | Drastic | NA |
| | G0000006719 | V9083837 | | | | nonsynonymous | |
| MUC4 | CMX- | CMX- | chr3: 195507422 | C | G | Drastic | NA |
| | G0000006719 | V9083838 | | | | nonsynonymous | |
| MUC4 | CMX- | CMX- | chr3: 195507428 | T | A | Drastic | NA |
| | G0000006719 | V9083839 | | | | nonsynonymous | |
| MUC4 | CMX- | CMX- | chr3: 195507433 | G | A | Drastic | NA |
| | G0000006719 | V9083840 | | | | nonsynonymous | |
| MUC4 | CMX- | CMX- | chr3: 195507434 | C | A | Drastic | NA |
| | G0000006719 | V9083841 | | | | nonsynonymous | |
| MUC4 | CMX- | CMX- | chr3: 195507443 | T | G | Drastic | NA |
| | G0000006719 | V9083842 | | | | nonsynonymous | |
| MUC4 | CMX- | CMX- | chr3: 195507445 | T | A | Drastic | NA |
| | G0000006719 | V9083843 | | | | nonsynonymous | |
| MUC4 | CMX- | CMX- | chr3: 195507446 | C | T | Drastic | NA |
| | G0000006719 | V9083844 | | | | nonsynonymous | |
| MUC4 | CMX- | CMX- | chr3: 195507461 | G | A | Drastic | NA |
| | G0000006719 | V9083845 | | | | nonsynonymous | |
| MUC4 | CMX- | CMX- | chr3: 195507475 | G | C | Drastic | NA |
| | G0000006719 | V9083846 | | | | nonsynonymous | |
| MUC4 | CMX- | CMX- | chr3: 195507491 | C | T | Drastic | NA |
| | G0000006719 | V9083847 | | | | nonsynonymous | |
| MUC4 | CMX- | CMX- | chr3: 195507494 | C | T | Drastic | NA |
| | G0000006719 | V9083848 | | | | nonsynonymous | |
| MUC4 | CMX- | CMX- | chr3: 195507502 | A | G | Drastic | NA |
| | G0000006719 | V9083849 | | | | nonsynonymous | |
| MUC4 | CMX- | CMX- | chr3: 195507604 | C | G | Drastic | NA |
| | G0000006719 | V9083850 | | | | nonsynonymous | |
| MUC4 | CMX- | CMX- | chr3: 195507605 | G | A | Drastic | NA |
| | G0000006719 | V9083851 | | | | nonsynonymous | |
| MUC4 | CMX- | CMX- | chr3: 195507614 | C | G | Drastic | NA |
| | G0000006719 | V9083852 | | | | nonsynonymous | |
| MUC4 | CMX- | CMX- | chr3: 195507620 | T | A | Drastic | NA |
| | G0000006719 | V9083853 | | | | nonsynonymous | |
| MUC4 | CMX- | CMX- | chr3: 195507625 | G | A | Drastic | NA |
| | G0000006719 | V9083854 | | | | nonsynonymous | |
| MUC4 | CMX- | CMX- | chr3: 195507635 | T | G | Drastic | NA |
| | G0000006719 | V9083855 | | | | nonsynonymous | |
| MUC4 | CMX- | CMX- | chr3: 195507077 | G | A | Drastic | NA |
| | G0000006719 | V9083856 | | | | nonsynonymous | |
| MUC4 | CMX- | CMX- | chr3: 195507694 | A | G | Drastic | NA |
| | G0000006719 | V9083857 | | | | nonsynonymous | |
| MUC4 | CMX- | CMX- | chr3: 195507731 | G | A | Drastic | NA |
| | G0000006719 | V9083858 | | | | nonsynonymous | |
| MUC4 | CMX- | CMX- | chr3: 195507779 | C | T | Drastic | NA |
| | G0000006719 | V9083859 | | | | nonsynonymous | |
| MUC4 | CMX- | CMX- | chr3: 195507790 | G | A | Drastic | NA |
| | G0000006719 | V9083860 | | | | nonsynonymous | |
| MUC4 | CMX- | CMX- | chr3: 195507827 | G | A | Drastic | NA |
| | G0000006719 | V9083861 | | | | nonsynonymous | |
| MUC4 | CMX- | CMX- | chr3: 195474159 | G | A | Drastic | NA |
| | G0000006719 | V9083862 | | | | nonsynonymous | |
| MUC4 | CMX- | CMX- | chr3: 195477786 | C | T | Drastic | NA |
| | G0000006719 | V9083863 | | | | nonsynonymous | |
| MUC4 | CMX- | CMX- | chr3: 195489009 | C | A | Drastic | NA |
| | G0000006719 | V9083864 | | | | nonsynonymous | |
| MUC4 | CMX- | CMX- | chr3: 195508019 | G | C | Drastic | NA |
| | G0000006719 | V9083865 | | | | nonsynonymous | |
| MUC4 | CMX- | CMX- | chr3: 195508021 | C | T | Drastic | NA |
| | G0000006719 | V9083866 | | | | nonsynonymous | |
| MUC4 | CMX- | CMX- | chr3: 195508069 | T | C | Drastic | NA |
| | G0000006719 | V9083867 | | | | nonsynonymous | |
| MUC4 | CMX- | CMX- | chr3: 195508070 | C | T | Drastic | NA |
| | G0000006719 | V9083868 | | | | nonsynonymous | |
| MUC4 | CMX- | CMX- | chr3: 195508091 | T | C | Drastic | NA |
| | G0000006719 | V9083869 | | | | nonsynonymous | |
| MUC4 | CMX- | CMX- | chr3: 195505886 | C | G | Drastic | NA |
| | G0000006719 | V9083870 | | | | nonsynonymous | |
| MUC4 | CMX- | CMX- | chr3: 195508115 | T | G | Drastic | NA |
| | G0000006719 | V9083871 | | | | nonsynonymous | |
| MUC4 | CMX- | CMX- | chr3: 195508127 | G | C | Drastic | NA |
| | G0000006719 | V9083872 | | | | nonsynonymous | |
| MUC4 | CMX- | CMX- | chr3: 195505907 | T | G | Drastic | NA |
| | G0000006719 | V9083873 | | | | nonsynonymous | |
| MUC4 | CMX- | CMX- | chr3: 195505930 | C | G | Drastic | NA |
| | G0000006719 | V9083874 | | | | nonsynonymous | |
| MUC4 | CMX- | CMX- | chr3: 195505955 | C | T | Drastic | NA |
| | G0000006719 | V9083875 | | | | nonsynonymous | |
| MUC4 | CMX- | CMX- | chr3: 195508336 | C | T | Drastic | NA |
| | G0000006719 | V9083876 | | | | nonsynonymous | |
| MUC4 | CMX- | CMX- | chr3: 195505979 | T | C | Drastic | NA |
| | G0000006719 | V9083877 | | | | nonsynonymous | |
| MUC4 | CMX- | CMX- | chr3: 195508451 | G | T | Drastic | NA |
| | G0000006719 | V9083878 | | | | nonsynonymous | |
| MUC4 | CMX- | CMX- | chr3: 195508453 | C | T | Drastic | NA |
| | G0000006719 | V9083879 | | | | nonsynonymous | |
| MUC4 | CMX- | CMX- | chr3: 195508475 | C | T | Drastic | NA |
| | G0000006719 | V9083880 | | | | nonsynonymous | |
| MUC4 | CMX- | CMX- | chr3: 195508478 | G | C | Drastic | NA |
| | G0000006719 | V9083881 | | | | nonsynonymous | |
| MUC4 | CMX- | CMX- | chr3: 195508500 | G | C | Drastic | NA |
| | G0000006719 | V9083882 | | | | nonsynonymous | |
| MUC4 | CMX- | CMX- | chr3: 195508501 | T | C | Drastic | NA |
| | G0000006719 | V9083883 | | | | nonsynonymous | |
| MUC4 | CMX- | CMX- | chr3: 195508502 | C | T | Drastic | NA |
| | G0000006719 | V9083884 | | | | nonsynonymous | |
| MUC4 | CMX- | CMX- | chr3: 195508523 | C | T | Drastic | NA |
| | G0000006719 | V9083885 | | | | nonsynonymous | |
| MUC4 | CMX- | CMX- | chr3: 195508526 | G | C | Drastic | NA |
| | G0000006719 | V9083886 | | | | nonsynonymous | |
| MUC4 | CMX- | CMX- | chr3: 195508667 | T | C | Drastic | NA |
| | G0000006719 | V9083887 | | | | nonsynonymous | |
| MUC4 | CMX- | CMX- | chr3: 195508668 | G | C | Drastic | NA |
| | G0000006719 | V9083888 | | | | nonsynonymous | |
| MUC4 | CMX- | CMX- | chr3: 195508702 | G | A | Drastic | NA |
| | G0000006719 | V9083889 | | | | nonsynonymous | |
| MUC4 | CMX- | CMX- | chr3: 195506311 | G | C | Drastic | NA |
| | G0000006719 | V9083890 | | | | nonsynonymous | |
| MUC4 | CMX- | CMX- | chr3: 195506315 | T | C | Drastic | NA |
| | G0000006719 | V9083891 | | | | nonsynonymous | |
| MUC4 | CMX- | CMX- | chr3: 195508787 | G | T | Drastic | NA |
| | G0000006719 | V9083892 | | | | nonsynonymous | |
| MUC4 | CMX- | CMX- | chr3: 195508789 | C | T | Drastic | NA |
| | G0000006719 | V9083893 | | | | nonsynonymous | |
| MUC4 | CMX- | CMX- | chr3: 195509029 | C | T | Drastic | NA |
| | G0000006719 | V9083894 | | | | nonsynonymous | |
| MUC4 | CMX- | CMX- | chr3: 195509093 | G | A | Drastic | NA |
| | G0000006719 | V9083895 | | | | nonsynonymous | |
| MUC4 | CMX- | CMX- | chr3: 195509099 | T | C | Drastic | NA |
| | G0000006719 | V9083896 | | | | nonsynonymous | |
| MUC4 | CMX- | CMX- | chr3: 195509102 | G | C | Drastic | NA |
| | G0000006719 | V9083897 | | | | nonsynonymous | |
| MUC4 | CMX- | CMX- | chr3: 195506389 | C | T | Drastic | NA |
| | G0000006719 | V9083898 | | | | nonsynonymous | |
| MUC4 | CMX- | CMX- | chr3: 195509212 | G | A | Drastic | NA |
| | G0000006719 | V9083899 | | | | nonsynonymous | |
| MUC4 | CMX- | CMX- | chr3: 195509287 | T | G | Drastic | NA |
| | G0000006719 | V9083900 | | | | nonsynonymous | |
| MUC4 | CMX- | CMX- | chr3: 195509353 | G | A | Drastic | NA |
| | G0000006719 | V9083901 | | | | nonsynonymous | |
| MUC4 | CMX- | CMX- | chr3: 195509354 | C | T | Drastic | NA |
| | G0000006719 | V9083902 | | | | nonsynonymous | |
| MUC4 | CMX- | CMX- | chr3: 195509363 | G | T | Drastic | NA |
| | G0000006719 | V9083903 | | | | nonsynonymous | |
| MUC4 | CMX- | CMX- | chr3: 195509365 | C | T | Drastic | NA |
| | G0000006719 | V9083904 | | | | nonsynonymous | |
| MUC4 | CMX- | CMX- | chr3: 195509374 | T | G | Drastic | NA |
| | G0000006719 | V9083905 | | | | nonsynonymous | |
| MUC4 | CMX- | CMX- | chr3: 195509378 | G | C | Drastic | NA |
| | G0000006719 | V9083906 | | | | nonsynonymous | |
| MUC4 | CMX- | CMX- | chr3: 195509423 | G | A | Drastic | NA |
| | G0000006719 | V9083907 | | | | nonsynonymous | |
| MUC4 | CMX- | CMX- | chr3: 195506554 | G | A | Drastic | NA |
| | G0000006719 | V9083908 | | | | nonsynonymous | |
| MUC4 | CMX- | CMX- | chr3: 195509563 | A | T | Drastic | NA |
| | G0000006719 | V9083909 | | | | nonsynonymous | |
| MUC4 | CMX- | CMX- | chr3: 195509573 | A | G | Drastic | NA |
| | G0000006719 | V9083910 | | | | nonsynonymous | |
| MUC4 | CMX- | CMX- | chr3: 195509606 | C | T | Drastic | NA |
| | G0000006719 | V9083911 | | | | nonsynonymous | |
| MUC4 | CMX- | CMX- | chr3: 195506617 | G | A | Drastic | NA |
| | G0000006719 | V9083912 | | | | nonsynonymous | |
| MUC4 | CMX- | CMX- | chr3: 195509627 | T | C | Drastic | NA |
| | G0000006719 | V9083913 | | | | nonsynonymous | |
| MUC4 | CMX- | CMX- | chr3: 195509651 | G | A | Drastic | NA |
| | G0000006719 | V9083914 | | | | nonsynonymous | |
| MUC4 | CMX- | CMX- | chr3: 195509756 | G | C | Drastic | NA |
| | G0000006719 | V9083915 | | | | nonsynonymous | |
| MUC4 | CMX- | CMX- | chr3: 195509795 | C | T | Drastic | NA |
| | G0000006719 | V9083916 | | | | nonsynonymous | |
| MUC4 | CMX- | CMX- | chr3: 195509861 | A | G | Drastic | NA |
| | G0000006719 | V9083917 | | | | nonsynonymous | |
| MUC4 | CMX- | CMX- | chr3: 195509879 | A | G | Drastic | NA |
| | G0000006719 | V9083918 | | | | nonsynonymous | |
| MUC4 | CMX- | CMX- | chr3: 195509918 | G | C | Drastic | NA |
| | G0000006719 | V9083919 | | | | nonsynonymous | |
| MUC4 | CMX- | CMX- | chr3: 195509939 | G | T | Drastic | NA |
| | G0000006719 | V9083920 | | | | nonsynonymous | |
| MUC4 | CMX- | CMX- | chr3: 195509941 | A | C | Drastic | NA |
| | G0000006719 | V9083921 | | | | nonsynonymous | |
| MUC4 | CMX- | CMX- | chr3: 195509954 | G | C | Drastic | NA |
| | G0000006719 | V9083922 | | | | nonsynonymous | |
| MUC4 | CMX- | CMX- | chr3: 195509957 | A | G | Drastic | NA |
| | G0000006719 | V9083923 | | | | nonsynonymous | |
| MUC4 | CMX- | CMX- | chr3: 195509974 | A | G | Drastic | NA |
| | G0000006719 | V9083924 | | | | nonsynonymous | |
| MUC4 | CMX- | CMX- | chr3: 195510068 | T | A | Drastic | NA |
| | G0000006719 | V9083925 | | | | nonsynonymous | |
| MUC4 | CMX- | CMX- | chr3: 195510083 | G | T | Drastic | NA |
| | G0000006719 | V9083926 | | | | nonsynonymous | |
| MUC4 | CMX- | CMX- | chr3: 195510146 | G | C | Drastic | NA |
| | G0000006719 | V9083927 | | | | nonsynonymous | |
| MUC4 | CMX- | CMX- | chr3: 195510194 | G | C | Drastic | NA |
| | G0000006719 | V9083928 | | | | nonsynonymous | |
| MUC4 | CMX- | CMX- | chr3: 195510590 | C | G | Drastic | NA |
| | G0000006719 | V9083929 | | | | nonsynonymous | |
| MUC4 | CMX- | CMX- | chr3: 195506983 | G | A | Drastic | NA |
| | G0000006719 | V9083930 | | | | nonsynonymous | |
| MUC4 | CMX- | CMX- | chr3: 195510655 | T | G | Drastic | NA |
| | G0000006719 | V9083931 | | | | nonsynonymous | |
| MUC4 | CMX- | CMX- | chr3: 195510659 | T | C | Drastic | NA |
| | G0000006719 | V9083932 | | | | nonsynonymous | |
| MUC4 | CMX- | CMX- | chr3: 195510662 | C | T | Drastic | NA |
| | G0000006719 | V9083933 | | | | nonsynonymous | |
| MUC4 | CMX- | CMX- | chr3: 195510683 | T | C | Drastic | NA |
| | G0000006719 | V9083934 | | | | nonsynonymous | |
| MUC4 | CMX- | CMX- | chr3: 195510686 | C | G | Drastic | NA |
| | G0000006719 | V9083935 | | | | nonsynonymous | |
| MUC4 | CMX- | CMX- | chr3: 195510697 | G | A | Drastic | NA |
| | G0000006719 | V9083936 | | | | nonsynonymous | |
| MUC4 | CMX- | CMX- | chr3: 195510706 | G | A | Drastic | NA |
| | G0000006719 | V9083937 | | | | nonsynonymous | |
| MUC4 | CMX- | CMX- | chr3: 195510707 | T | G | Drastic | NA |
| | G0000006719 | V9083938 | | | | nonsynonymous | |
| MUC4 | CMX- | CMX- | chr3: 195510709 | C | T | Drastic | NA |
| | G0000006719 | V9083939 | | | | nonsynonymous | |
| MUC4 | CMX- | CMX- | chr3: 195510718 | G | T | Drastic | NA |
| | G0000006719 | V9083940 | | | | nonsynonymous | |
| MUC4 | CMX- | CMX- | chr3: 195510745 | G | A | Drastic | NA |
| | G0000006719 | V9083941 | | | | nonsynonymous | |
| MUC4 | CMX- | CMX- | chr3: 195510749 | C | A | Drastic | NA |
| | G0000006719 | V9083942 | | | | nonsynonymous | |
| MUC4 | CMX- | CMX- | chr3: 195510766 | G | T | Drastic | NA |
| | G0000006719 | V9083943 | | | | nonsynonymous | |
| MUC4 | CMX- | CMX- | chr3: 195510767 | G | A | Drastic | NA |
| | G0000006719 | V9083944 | | | | nonsynonymous | |
| MUC4 | CMX- | CMX- | chr3: 195510773 | A | G | Drastic | NA |
| | G0000006719 | V9083945 | | | | nonsynonymous | |
| MUC4 | CMX- | CMX- | chr3: 195510827 | C | T | Drastic | NA |
| | G0000006719 | V9083946 | | | | nonsynonymous | |
| MUC4 | CMX- | CMX- | chr3: 195510896 | G | A | Drastic | NA |
| | G0000006719 | V9083947 | | | | nonsynonymous | |
| MUC4 | CMX- | CMX- | chr3: 195510899 | T | C | Drastic | NA |
| | G0000006719 | V9083948 | | | | nonsynonymous | |
| MUC4 | CMX- | CMX- | chr3: 195510910 | G | T | Drastic | NA |
| | G0000006719 | V9083949 | | | | nonsynonymous | |
| MUC4 | CMX- | CMX- | chr3: 195510943 | G | T | Drastic | NA |
| | G0000006719 | V9083950 | | | | nonsynonymous | |
| MUC4 | CMX- | CMX- | chr3: 195511013 | G | A | Drastic | NA |
| | G0000006719 | V9083951 | | | | nonsynonymous | |
| MUC4 | CMX- | CMX- | chr3: 195511019 | T | C | Drastic | NA |
| | G0000006719 | V9083952 | | | | nonsynonymous | |
| MUC4 | CMX- | CMX- | chr3: 195511043 | T | C | Drastic | NA |
| | G0000006719 | V9083953 | | | | nonsynonymous | |
| MUC4 | CMX- | CMX- | chr3: 195511051 | C | A | Drastic | NA |
| | G0000006719 | V9083954 | | | | nonsynonymous | |
| MUC4 | CMX- | CMX- | chr3: 195511070 | C | G | Drastic | NA |
| | G0000006719 | V9083955 | | | | nonsynonymous | |
| MUC4 | CMX- | CMX- | chr3: 195511076 | T | A | Drastic | NA |
| | G0000006719 | V9083956 | | | | nonsynonymous | |
| MUC4 | CMX- | CMX- | chr3: 195511102 | G | A | Drastic | NA |
| | G0000006719 | V9083957 | | | | nonsynonymous | |
| MUC4 | CMX- | CMX- | chr3: 195511142 | T | C | Drastic | NA |
| | G0000006719 | V9083958 | | | | nonsynonymous | |
| MUC4 | CMX- | CMX- | chr3: 195511156 | C | G | Drastic | NA |
| | G0000006719 | V9083959 | | | | nonsynonymous | |
| MUC4 | CMX- | CMX- | chr3: 195511186 | A | G | Drastic | NA |
| | G0000006719 | V9083960 | | | | nonsynonymous | |
| MUC4 | CMX- | CMX- | chr3: 195511190 | C | T | Drastic | NA |
| | G0000006719 | V9083961 | | | | nonsynonymous | |
| MUC4 | CMX- | CMX- | chr3: 195511204 | T | G | Drastic | NA |
| | G0000006719 | V9083962 | | | | nonsynonymous | |
| MUC4 | CMX- | CMX- | chr3: 195511211 | C | T | Drastic | NA |
| | G0000006719 | V9083963 | | | | nonsynonymous | |
| MUC4 | CMX- | CMX- | chr3: 195511214 | G | C | Drastic | NA |
| | G0000006719 | V9083964 | | | | nonsynonymous | |
| MUC4 | CMX- | CMX- | chr3: 195511268 | T | A | Drastic | NA |
| | G0000006719 | V9083965 | | | | nonsynonymous | |
| MUC4 | CMX- | CMX- | chr3: 195511273 | G | A | Drastic | NA |
| | G0000006719 | V9083966 | | | | nonsynonymous | |
| MUC4 | CMX- | CMX- | chr3: 195511285 | T | C | Drastic | NA |
| | G0000006719 | V9083967 | | | | nonsynonymous | |
| MUC4 | CMX- | CMX- | chr3: 195511286 | C | T | Drastic | NA |
| | G0000006719 | V9083968 | | | | nonsynonymous | |
| MUC4 | CMX- | CMX- | chr3: 195511331 | A | G | Drastic | NA |
| | G0000006719 | V9083969 | | | | nonsynonymous | |
| MUC4 | CMX- | CMX- | chr3: 195511336 | G | C | Drastic | NA |
| | G0000006719 | V9083970 | | | | nonsynonymous | |
| MUC4 | CMX- | CMX- | chr3: 195511358 | C | G | Drastic | NA |
| | G0000006719 | V9083971 | | | | nonsynonymous | |
| MUC4 | CMX- | CMX- | chr3: 195511390 | T | G | Drastic | NA |
| | G0000006719 | V9083972 | | | | nonsynonymous | |
| MUC4 | CMX- | CMX- | chr3: 195511396 | G | A | Drastic | NA |
| | G0000006719 | V9083973 | | | | nonsynonymous | |
| MUC4 | CMX- | CMX- | chr3: 195511403 | C | T | Drastic | NA |
| | G0000006719 | V9083974 | | | | nonsynonymous | |
| MUC4 | CMX- | CMX- | chr3: 195511412 | T | A | Drastic | NA |
| | G0000006719 | V9083975 | | | | nonsynonymous | |
| MUC4 | CMX- | CMX- | chr3: 195511438 | G | T | Drastic | NA |
| | G0000006719 | V9083976 | | | | nonsynonymous | |
| MUC4 | CMX- | CMX- | chr3: 195507683 | C | T | Drastic | NA |
| | G0000006719 | V9083977 | | | | nonsynonymous | |
| MUC4 | CMX- | CMX- | chr3: 195511454 | C | G | Drastic | NA |
| | G0000006719 | V9083978 | | | | nonsynonymous | |
| MUC4 | CMX- | CMX- | chr3: 195511460 | T | A | Drastic | NA |
| | G0000006719 | V9083979 | | | | nonsynonymous | |
| MUC4 | CMX- | CMX- | chr3: 195511465 | G | A | Drastic | NA |
| | G0000006719 | V9083980 | | | | nonsynonymous | |
| MUC4 | CMX- | CMX- | chr3: 195511474 | A | G | Drastic | NA |
| | G0000006719 | V9083981 | | | | nonsynonymous | |
| MUC4 | CMX- | CMX- | chr3: 195511486 | G | T | Drastic | NA |
| | G0000006719 | V9083982 | | | | nonsynonymous | |
| MUC4 | CMX- | CMX- | chr3: 195507925 | C | T | Drastic | NA |
| | G0000006719 | V9083983 | | | | nonsynonymous | |
| MUC4 | CMX- | CMX- | chr3: 195508009 | G | A | Drastic | NA |
| | G0000006719 | V9083984 | | | | nonsynonymous | |
| MUC4 | CMX- | CMX- | chr3: 195508010 | C | A | Drastic | NA |
| | G0000006719 | V9083985 | | | | nonsynonymous | |
| MUC4 | CMX- | CMX- | chr3: 195511513 | G | A | Drastic | NA |
| | G0000006719 | V9083986 | | | | nonsynonymous | |
| MUC4 | CMX- | CMX- | chr3: 195511525 | T | C | Drastic | NA |
| | G0000006719 | V9083987 | | | | nonsynonymous | |
| MUC4 | CMX- | CMX- | chr3: 195511526 | C | T | Drastic | NA |
| | G0000006719 | V9083988 | | | | nonsynonymous | |
| MUC4 | CMX- | CMX- | chr3: 195511534 | T | G | Drastic | NA |
| | G0000006719 | V9083989 | | | | nonsynonymous | |
| MUC4 | CMX- | CMX- | chr3: 195511547 | C | T | Drastic | NA |
| | G0000006719 | V9083990 | | | | nonsynonymous | |
| MUC4 | CMX- | CMX- | chr3: 195508108 | G | A | Drastic | NA |
| | G0000006719 | V9083991 | | | | nonsynonymous | |
| MUC4 | CMX- | CMX- | chr3: 195511690 | G | C | Drastic | NA |
| | G0000006719 | V9083992 | | | | nonsynonymous | |
| MUC4 | CMX- | CMX- | chr3: 195511705 | G | A | Drastic | NA |
| | G0000006719 | V9083993 | | | | nonsynonymous | |
| MUC4 | CMX- | CMX- | chr3: 195508175 | G | C | Drastic | NA |
| | G0000006719 | V9083994 | | | | nonsynonymous | |
| MUC4 | CMX- | CMX- | chr3: 195508178 | G | C | Drastic | NA |
| | G0000006719 | V9083995 | | | | nonsynonymous | |
| MUC4 | CMX- | CMX- | chr3: 195508238 | C | G | Drastic | NA |
| | G0000006719 | V9083996 | | | | nonsynonymous | |
| MUC4 | CMX- | CMX- | chr3: 195511822 | G | T | Drastic | NA |
| | G0000006719 | V9083997 | | | | nonsynonymous | |
| MUC4 | CMX- | CMX- | chr3: 195508402 | G | T | Drastic | NA |
| | G0000006719 | V9083998 | | | | nonsynonymous | |
| MUC4 | CMX- | CMX- | chr3: 195511870 | G | A | Drastic | NA |
| | G0000006719 | V9083999 | | | | nonsynonymous | |
| MUC4 | CMX- | CMX- | chr3: 195511877 | G | A | Drastic | NA |
| | G0000006719 | V9084000 | | | | nonsynonymous | |
| MUC4 | CMX- | CMX- | chr3: 195511918 | G | T | Drastic | NA |
| | G0000006719 | V9084001 | | | | nonsynonymous | |
| MUC4 | CMX- | CMX- | chr3: 195511925 | A | G | Drastic | NA |
| | G0000006719 | V9084002 | | | | nonsynonymous | |
| MUC4 | CMX- | CMX- | chr3: 195511937 | C | T | Drastic | NA |
| | G0000006719 | V9084003 | | | | nonsynonymous | |
| MUC4 | CMX- | CMX- | chr3: 195512042 | T | C | Drastic | NA |
| | G0000006719 | V9084004 | | | | nonsynonymous | |
| MUC4 | CMX- | CMX- | chr3: 195512107 | T | A | Drastic | NA |
| | G0000006719 | V9084005 | | | | nonsynonymous | |
| MUC4 | CMX- | CMX- | chr3: 195512117 | C | G | Drastic | NA |
| | G0000006719 | V9084006 | | | | nonsynonymous | |
| MUC4 | CMX- | CMX- | chr3: 195512195 | C | T | Drastic | NA |
| | G0000006719 | V9084007 | | | | nonsynonymous | |
| MUC4 | CMX- | CMX- | chr3: 195512206 | A | G | Drastic | NA |
| | G0000006719 | V9084008 | | | | nonsynonymous | |
| MUC4 | CMX- | CMX- | chr3: 195512212 | G | T | Drastic | NA |
| | G0000006719 | V9084009 | | | | nonsynonymous | |
| MUC4 | CMX- | CMX- | chr3: 195512242 | G | A | Drastic | NA |
| | G0000006719 | V9084010 | | | | nonsynonymous | |
| MUC4 | CMX- | CMX- | chr3: 195508774 | G | T | Drastic | NA |
| | G0000006719 | V9084011 | | | | nonsynonymous | |
| MUC4 | CMX- | CMX- | chr3: 195508786 | A | G | Drastic | NA |
| | G0000006719 | V9084012 | | | | nonsynonymous | |
| MUC4 | CMX- | CMX- | chr3: 195512267 | T | C | Drastic | NA |
| | G0000006719 | V9084013 | | | | nonsynonymous | |
| MUC4 | CMX- | CMX- | chr3: 195512270 | C | G | Drastic | NA |
| | G0000006719 | V9084014 | | | | nonsynonymous | |
| MUC4 | CMX- | CMX- | chr3: 195512287 | G | A | Drastic | NA |
| | G0000006719 | V9084015 | | | | nonsynonymous | |
| MUC4 | CMX- | CMX- | chr3: 195512302 | G | A | Drastic | NA |
| | G0000006719 | V9084016 | | | | nonsynonymous | |
| MUC4 | CMX- | CMX- | chr3: 195512567 | G | A | Drastic | NA |
| | G0000006719 | V9084017 | | | | nonsynonymous | |
| MUC4 | CMX- | CMX- | chr3: 195512597 | G | A | Drastic | NA |
| | G0000006719 | V9084018 | | | | nonsynonymous | |
| MUC4 | CMX- | CMX- | chr3: 195509170 | A | G | Drastic | NA |
| | G0000006719 | V9084019 | | | | nonsynonymous | |
| MUC4 | CMX- | CMX- | chr3: 195512606 | G | C | Drastic | NA |
| | G0000006719 | V9084020 | | | | nonsynonymous | |
| MUC4 | CMX- | CMX- | chr3: 195512665 | G | A | Drastic | NA |
| | G0000006719 | V9084021 | | | | nonsynonymous | |
| MUC4 | CMX- | CMX- | chr3: 195512686 | G | T | Drastic | NA |
| | G0000006719 | V9084022 | | | | nonsynonymous | |
| MUC4 | CMX- | CMX- | chr3: 195512693 | A | G | Drastic | NA |
| | G0000006719 | V9084023 | | | | nonsynonymous | |
| MUC4 | CMX- | CMX- | chr3: 195512767 | T | G | Drastic | NA |
| | G0000006719 | V9084024 | | | | nonsynonymous | |
| MUC4 | CMX- | CMX- | chr3: 195512768 | T | A | Drastic | NA |
| | G0000006719 | V9084025 | | | | nonsynonymous | |
| MUC4 | CMX- | CMX- | chr3: 195513136 | G | C | Drastic | NA |
| | G0000006719 | V9084026 | | | | nonsynonymous | |
| MUC4 | CMX- | CMX- | chr3: 195513154 | G | T | Drastic | NA |
| | G0000006719 | V9084027 | | | | nonsynonymous | |
| MUC4 | CMX- | CMX- | chr3: 195513155 | T | C | Drastic | NA |
| | G0000006719 | V9084028 | | | | nonsynonymous | |
| MUC4 | CMX- | CMX- | chr3: 195509476 | A | G | Drastic | NA |
| | G0000006719 | V9084029 | | | | nonsynonymous | |
| MUC4 | CMX- | CMX- | chr3: 195513203 | C | T | Drastic | NA |
| | G0000006719 | V9084030 | | | | nonsynonymous | |
| MUC4 | CMX- | CMX- | chr3: 195513214 | A | G | Drastic | NA |
| | G0000006719 | V9084031 | | | | nonsynonymous | |
| MUC4 | CMX- | CMX- | chr3: 195513364 | C | T | Drastic | NA |
| | G0000006719 | V9084032 | | | | nonsynonymous | |
| MUC4 | CMX- | CMX- | chr3: 195509614 | G | A | Drastic | NA |
| | G0000006719 | V9084033 | | | | nonsynonymous | |
| MUC4 | CMX- | CMX- | chr3: 195513383 | T | A | Drastic | NA |
| | G0000006719 | V9084034 | | | | nonsynonymous | |
| MUC4 | CMX- | CMX- | chr3: 195513394 | A | T | Drastic | NA |
| | G0000006719 | V9084035 | | | | nonsynonymous | |
| MUC4 | CMX- | CMX- | chr3: 195513395 | G | T | Drastic | NA |
| | G0000006719 | V9084036 | | | | nonsynonymous | |
| MUC4 | CMX- | CMX- | chr3: 195513397 | C | T | Drastic | NA |
| | G0000006719 | V9084037 | | | | nonsynonymous | |
| MUC4 | CMX- | CMX- | chr3: 195513398 | C | T | Drastic | NA |
| | G0000006719 | V9084038 | | | | nonsynonymous | |
| MUC4 | CMX- | CMX- | chr3: 195513413 | G | A | Drastic | NA |
| | G0000006719 | V9084039 | | | | nonsynonymous | |
| MUC4 | CMX- | CMX- | chr3: 195513433 | G | A | Drastic | NA |
| | G0000006719 | V9084040 | | | | nonsynonymous | |
| MUC4 | CMX- | CMX- | chr3: 195513442 | G | T | Drastic | NA |
| | G0000006719 | V9084041 | | | | nonsynonymous | |
| MUC4 | CMX- | CMX- | chr3: 195513445 | C | T | Drastic | NA |
| | G0000006719 | V9084042 | | | | nonsynonymous | |
| MUC4 | CMX- | CMX- | chr3: 195513461 | G | A | Drastic | NA |
| | G0000006719 | V9084043 | | | | nonsynonymous | |
| MUC4 | CMX- | CMX- | chr3: 195513491 | G | T | Drastic | NA |
| | G0000006719 | V9084044 | | | | nonsynonymous | |
| MUC4 | CMX- | CMX- | chr3: 195513502 | T | G | Drastic | NA |
| | G0000006719 | V9084045 | | | | nonsynonymous | |
| MUC4 | CMX- | CMX- | chr3: 195513515 | C | T | Drastic | NA |
| | G0000006719 | V9084046 | | | | nonsynonymous | |
| MUC4 | CMX- | CMX- | chr3: 195513598 | G | A | Drastic | NA |
| | G0000006719 | V9084047 | | | | nonsynonymous | |
| MUC4 | CMX- | CMX- | chr3: 195513667 | T | G | Drastic | NA |
| | G0000006719 | V9084048 | | | | nonsynonymous | |
| MUC4 | CMX- | CMX- | chr3: 195513743 | G | T | Drastic | NA |
| | G0000006719 | V9084049 | | | | nonsynonymous | |
| MUC4 | CMX- | CMX- | chr3: 195513779 | C | T | Drastic | NA |
| | G0000006719 | V9084050 | | | | nonsynonymous | |
| MUC4 | CMX- | CMX- | chr3: 195510649 | G | A | Drastic | NA |
| | G0000006719 | V9084051 | | | | nonsynonymous | |
| MUC4 | CMX- | CMX- | chr3: 195513991 | G | A | Drastic | NA |
| | G0000006719 | V9084052 | | | | nonsynonymous | |
| MUC4 | CMX- | CMX- | chr3: 195514109 | C | A | Drastic | NA |
| | G0000006719 | V9084053 | | | | nonsynonymous | |
| MUC4 | CMX- | CMX- | chr3: 195514144 | T | C | Drastic | NA |
| | G0000006719 | V9084054 | | | | nonsynonymous | |
| MUC4 | CMX- | CMX- | chr3: 195514324 | G | A | Drastic | NA |
| | G0000006719 | V9084055 | | | | nonsynonymous | |
| MUC4 | CMX- | CMX- | chr3: 195514379 | T | C | Drastic | NA |
| | G0000006719 | V9084056 | | | | nonsynonymous | |
| MUC4 | CMX- | CMX- | chr3: 195514403 | C | T | Drastic | NA |
| | G0000006719 | V9084057 | | | | nonsynonymous | |
| MUC4 | CMX- | CMX- | chr3: 195514643 | T | G | Drastic | NA |
| | G0000006719 | V9084058 | | | | nonsynonymous | |
| MUC4 | CMX- | CMX- | chr3: 195514645 | T | C | Drastic | NA |
| | G0000006719 | V9084059 | | | | nonsynonymous | |
| MUC4 | CMX- | CMX- | chr3: 195514646 | C | T | Drastic | NA |
| | G0000006719 | V9084060 | | | | nonsynonymous | |
| MUC4 | CMX- | CMX- | chr3: 195514654 | A | G | Drastic | NA |
| | G0000006719 | V9084061 | | | | nonsynonymous | |
| MUC4 | CMX- | CMX- | chr3: 195514661 | A | G | Drastic | NA |
| | G0000006719 | V9084062 | | | | nonsynonymous | |
| MUC4 | CMX- | CMX- | chr3: 195514718 | G | C | Drastic | NA |
| | G0000006719 | V9084063 | | | | nonsynonymous | |
| MUC4 | CMX- | CMX- | chr3: 195514729 | G | A | Drastic | NA |
| | G0000006719 | V9084064 | | | | nonsynonymous | |
| MUC4 | CMX- | CMX- | chr3: 195514733 | C | A | Drastic | NA |
| | G0000006719 | V9084065 | | | | nonsynonymous | |
| MUC4 | CMX- | CMX- | chr3: 195514741 | A | C | Drastic | NA |
| | G0000006719 | V9084066 | | | | nonsynonymous | |
| MUC4 | CMX- | CMX- | chr3: 195514757 | A | G | Drastic | NA |
| | G0000006719 | V9084067 | | | | nonsynonymous | |
| MUC4 | CMX- | CMX- | chr3: 195514805 | G | A | Drastic | NA |
| | G0000006719 | V9084068 | | | | nonsynonymous | |
| MUC4 | CMX- | CMX- | chr3: 195514811 | C | T | Drastic | NA |
| | G0000006719 | V9084069 | | | | nonsynonymous | |
| MUC4 | CMX- | CMX- | chr3: 195514812 | G | C | Drastic | NA |
| | G0000006719 | V9084070 | | | | nonsynonymous | |
| MUC4 | CMX- | CMX- | chr3: 195514825 | G | A | Drastic | NA |
| | G0000006719 | V9084071 | | | | nonsynonymous | |
| MUC4 | CMX- | CMX- | chr3: 195514846 | A | G | Drastic | NA |
| | G0000006719 | V9084072 | | | | nonsynonymous | |
| MUC4 | CMX- | CMX- | chr3: 195514859 | C | T | Drastic | NA |
| | G0000006719 | V9084073 | | | | nonsynonymous | |
| MUC4 | CMX- | CMX- | chr3: 195514862 | G | C | Drastic | NA |
| | G0000006719 | V9084074 | | | | nonsynonymous | |
| MUC4 | CMX- | CMX- | chr3: 195514873 | G | A | Drastic | NA |
| | G0000006719 | V9084075 | | | | nonsynonymous | |
| MUC4 | CMX- | CMX- | chr3: 195514882 | G | A | Drastic | NA |
| | G0000006719 | V9084076 | | | | nonsynonymous | |
| MUC4 | CMX- | CMX- | chr3: 195514930 | A | G | Drastic | NA |
| | G0000006719 | V9084077 | | | | nonsynonymous | |
| MUC4 | CMX- | CMX- | chr3: 195514948 | G | A | Drastic | NA |
| | G0000006719 | V9084078 | | | | nonsynonymous | |
| MUC4 | CMX- | CMX- | chr3: 195514969 | G | A | Drastic | NA |
| | G0000006719 | V9084079 | | | | nonsynonymous | |
| MUC4 | CMX- | CMX- | chr3: 195515003 | T | C | Drastic | NA |
| | G0000006719 | V9084080 | | | | nonsynonymous | |
| MUC4 | CMX- | CMX- | chr3: 195515008 | C | G | Drastic | NA |
| | G0000006719 | V9084081 | | | | nonsynonymous | |
| MUC4 | CMX- | CMX- | chr3: 195515038 | G | A | Drastic | NA |
| | G0000006719 | V9084082 | | | | nonsynonymous | |
| MUC4 | CMX- | CMX- | chr3: 195515045 | A | G | Drastic | NA |
| | G0000006719 | V9084083 | | | | nonsynonymous | |
| MUC4 | CMX- | CMX- | chr3: 195515122 | G | C | Drastic | NA |
| | G0000006719 | V9084084 | | | | nonsynonymous | |
| MUC4 | CMX- | CMX- | chr3: 195515134 | G | T | Drastic | NA |
| | G0000006719 | V9084085 | | | | nonsynonymous | |
| MUC4 | CMX- | CMX- | chr3: 195515141 | A | G | Drastic | NA |
| | G0000006719 | V9084086 | | | | nonsynonymous | |
| MUC4 | CMX- | CMX- | chr3: 195515194 | G | C | Drastic | NA |
| | G0000006719 | V9084087 | | | | nonsynonymous | |
| MUC4 | CMX- | CMX- | chr3: 195515387 | T | C | Drastic | NA |
| | G0000006719 | V9084088 | | | | nonsynonymous | |
| MUC4 | CMX- | CMX- | chr3: 195515411 | G | T | Drastic | NA |
| | G0000006719 | V9084089 | | | | nonsynonymous | |
| MUC4 | CMX- | CMX- | chr3: 195515413 | C | T | Drastic | NA |
| | G0000006719 | V9084090 | | | | nonsynonymous | |
| MUC4 | CMX- | CMX- | chr3: 195515449 | A | T | Drastic | NA |
| | G0000006719 | V9084091 | | | | nonsynonymous | |
| MUC4 | CMX- | CMX- | chr3: 195515459 | C | T | Drastic | NA |
| | G0000006719 | V9084092 | | | | nonsynonymous | |
| MUC4 | CMX- | CMX- | chr3: 195538901 | C | T | Start codon | NA |
| | G0000006719 | V9084093 | | | | gained | |
| MUC4 | CMX- | CMX- | chr3: 195512246 | T | C | Drastic | NA |
| | G0000006719 | V9084094 | | | | nonsynonymous | |
| MUC4 | CMX- | CMX- | chr3: 195511556 | T | A | Drastic | NA |
| | G0000006719 | V9084095 | | | | nonsynonymous | |
| MUC4 | CMX- | CMX- | chr3: 195512603 | T | C | Drastic | NA |
| | G0000006719 | V9084096 | | | | nonsynonymous | |
| MUC4 | CMX- | CMX- | chr3: 195513173 | G | A | Drastic | NA |
| | G0000006719 | V9084097 | | | | nonsynonymous | |
| MUC4 | CMX- | CMX- | chr3: 195511451 | T | C | Drastic | NA |
| | G0000006719 | V9084098 | | | | nonsynonymous | |
| MUC4 | CMX- | CMX- | chr3: 195511781 | G | A | Drastic | NA |
| | G0000006719 | V9084099 | | | | nonsynonymous | |
| MUC4 | CMX- | CMX- | chr3: 195511499 | C | T | Drastic | NA |
| | G0000006719 | V9084100 | | | | nonsynonymous | |
| MUC4 | CMX- | CMX- | chr3: 195513365 | G | A | Drastic | NA |
| | G0000006719 | V9084101 | | | | nonsynonymous | |
| MUC4 | CMX- | CMX- | chr3: 195511780 | G | A | Drastic | NA |
| | G0000006719 | V9084102 | | | | nonsynonymous | |
| MUC4 | CMX- | CMX- | chr3: 195513826 | G | A | Drastic | NA |
| | G0000006719 | V9084103 | | | | nonsynonymous | |
| MUC4 | CMX- | CMX- | chr3: 195512245 | T | C | Drastic | NA |
| | G0000006719 | V9084104 | | | | nonsynonymous | |
| MUC4 | CMX- | CMX- | chr3: 195511500 | G | C | Drastic | NA |
| | G0000006719 | V9084105 | | | | nonsynonymous | |
| MUC4 | CMX- | CMX- | chr3: 195511502 | G | C | Drastic | NA |
| | G0000006719 | V9084106 | | | | nonsynonymous | |
| MUC4 | CMX- | CMX- | chr3: 195511859 | T | G | Drastic | NA |
| | G0000006719 | V9084107 | | | | nonsynonymous | |
| MUC4 | CMX- | CMX- | chr3: 195511783 | A | G | Drastic | NA |
| | G0000006719 | V9084108 | | | | nonsynonymous | |
| MUC4 | CMX- | CMX- | chr3: 195512373 | G | GG | Codon change | NA |
| | G0000006719 | SV00002 | | | AT | and codon | |
| | | | | | | insertion | |
| MUC4 | CMX- | CMX- | chr3: 195518112 | T | TGT | Codon change | NA |
| | G0000006719 | SV00003 | | | CTC | and codon | |
| | | | | | CTG | insertion | |
| | | | | | CGT | | |
| | | | | | AA | | |
| | | | | | CA | | |
| MUC4 | CMX- | CMX- | chr3: 195464985 | CNV | NA | Splice acceptor | NA |
| | G0000006719 | SV00004 | | duplication | | variant | |
| MUC4 | CMX- | CMX- | chr3: 195507809 | CNV | NA | Nonsynonymous | NA |
| | G0000006719 | SV00005 | | deletion | | and coding | |
| | | | | | | sequence | |
| MUC4 | CMX- | CMX- | chr3: 195508499 | CNV | NA | Frameshift | NA |
| | G0000006719 | SV00006 | | duplication | | | |
| MUC4 | CMX- | CMX- | chr3: 195499847 | A | G | NA | 6.75E−05 |
| | G0000006719 | V9084187 | | | | | |
| MUC4 | CMX- | CMX- | chr3: 195500367 | A | G | NA | 0.000532509 |
| | G0000006719 | V9084188 | | | | | |
| MUC4 | CMX- | CMX- | chr3: 195506750 | G | C | NA | 0.000425548 |
| | G0000006719 | V9084191 | | | | | |
| MUC4 | CMX- | CMX- | chr3: 195506760 | T | A | NA | 7.68E−05 |
| | G0000006719 | V9084192 | | | | | |
| MUC4 | CMX- | CMX- | chr3: 195506195 | C | T | NA | 8.00E−05 |
| | G0000006719 | V9084189 | | | | | |
| MUC4 | CMX- | CMX- | chr3: 195506746 | G | A | NA | 0.000150373 |
| | G0000006719 | V9084190 | | | | | |
| NLRP11 | CMX- | CMX- | chr19: 56320663 | G | A | Drastic | NA |
| | G0000028188 | V9084110 | | | | nonsynonymous | |
| NLRP11 | CMX- | CMX- | chr19: 56329447 | G | A | Drastic | NA |
| | G0000028188 | V9084111 | | | | nonsynonymous | |
| NLRP11 | CMX- | CMX- | chr19: 56343378 | C | A | Start codon | NA |
| | G0000028188 | V9084112 | | | | gained | |
| NLRP14 | CMX- | CMX- | chr11: 7091569 | C | T | Drastic | NA |
| | G0000016919 | V9084115 | | | | nonsynonymous | |
| NLRP14 | CMX- | CMX- | chr11: 7079038 | G | A | Drastic | NA |
| | G0000016919 | V9084116 | | | | nonsynonymous | |
| NLRP14 | CMX- | CMX- | chr11: 7059981 | G | A | Drastic | NA |
| | G0000016919 | V9084117 | | | | nonsynonymous | |
| NLRP5 | CMX- | CMX- | chr19: 56569629 | C | G | Drastic | NA |
| | G0000028192 | V9084120 | | | | nonsynonymous | |
| NLRP5 | CMX- | CMX- | chr19: 56572875 | G | A | Drastic | NA |
| | G0000028192 | V9084121 | | | | nonsynonymous | |
| NLRP5 | CMX- | CMX- | chr19: 56567147 | A | G | NA | 8.96E−06 |
| | G0000028192 | V9084170 | | | | | |
| NLRP5 | CMX- | CMX- | chr19: 56567133 | A | G | NA | 0.000422755 |
| | G0000028192 | V9084169 | | | | | |
| NLRP8 | CMX- | CMX- | chr19: 56459342 | C | T | Drastic | NA |
| | G0000028191 | V9084122 | | | | nonsynonymous | |
| NLRP8 | CMX- | CMX- | chr19: 56467375 | C | T | Drastic | NA |
| | G0000028191 | V9084123 | | | | nonsynonymous | |
| NLRP8 | CMX- | CMX- | chr19: 56499279 | G | C | Stop codon | NA |
| | G0000028191 | V9084124 | | | | lost | |
| PADI3 | CMX- | CMX- | chr1: 17548826-18037716 | NA | CNV | PADI3 (16 | NA |
| | G0000000342 | V1792728 | | | gain | exons) | |
| PADI6 | CMX- | CMX- | chr1: 17707931 | T | G | NA | 0.000947202 |
| | G0000000344 | V9084147 | | | | | |
| PADI6 | CMX- | CMX- | chr1: 17707757 | C | T | NA | 0.000791492 |
| | G0000000344 | V9084145 | | | | | |
| PADI6 | CMX- | CMX- | chr1: 17707758 | G | C | NA | 0.000832422 |
| | G0000000344 | V9084146 | | | | | |
| PAEP | CMX- | CMX- | chr9: 138131476-138644038 | NA | CNV | PAEP (2 | NA |
| | G0000015254 | V1271620 | | | gain | exons) | |
| PLCB1 | CMX- | CMX- | chr20: 8142398-10362561 | NA | CNV | PLCB1 (2 | NA |
| | G0000028445 | V1930635 | | | gain | exons) | |
| PMS2 | CMX- | CMX- | chr7: 6045627 | C | T | Drastic | NA |
| | G0000011251 | V9084128 | | | | nonsynonymous | |
| PMS2 | CMX- | CMX- | chr7: 6029313 | CNV | NA | Splice donor, | NA |
| | G0000011251 | SV00007 | | duplication | | acceptor and | |
| | | | | | | coding | |
| | | | | | | sequence | |
| PMS2 | CMX- | CMX- | chr7: 5981433 | A | G | NA | 0.000681822 |
| | G0000011251 | V9084222 | | | | | |
| POF1B | CMX- | CMX- | chrX: 77243971-85734966 | NA | CNV | POF1B (15 | NA |
| | G0000031099 | V1507096 | | | gain | exons) | |
| PRDM9 | CMX- | CMX- | chr5: 21969693-23940832 | NA | CNV | PRDM9 (3 | NA |
| | G0000008219 | V1222200 | | | loss | exons) | |
| SCARB1 | CMX- | CMX- | chr12: 125270773 | A | G | Drastic | NA |
| | G0000019991 | V9084131 | | | | nonsynonymous | |
| SCARB1 | CMX- | CMX- | chr12: 125323962 | A | C | Start codon | NA |
| | G0000019991 | V9084132 | | | | gained | |
| SCARB1 | CMX- | CMX- | chr12: 125324570 | C | T | Start codon | NA |
| | G0000019991 | V9084133 | | | | gained | |
| SCARB1 | CMX- | CMX- | chr12: 125324553 | C | T | Start codon | NA |
| | G0000019991 | V9084134 | | | | gained | |
| SERPINA10 | CMX- | CMX- | chr14: 94691918-95251285 | NA | CNV | SERPINA10 | NA |
| | G0000021629 | V1143735 | | | gain | (4 exons) | |
| SIRT3 | CMX- | CMX- | chr11: 222921-278027 | NA | CNV | SIRT3 (2 | NA |
| | G0000016629 | V1733950 | | | loss | exons) | |
| SPIN1 | CMX- | CMX- | chr9: 90754700 | G | A | NA | 0.000183378 |
| | G0000014689 | V9084227 | | | | | |
| SPIN1 | CMX- | CMX- | chr9: 90754733 | A | C | NA | 0.000548473 |
| | G0000014689 | V9084228 | | | | | |
| SPIN1 | CMX- | CMX- | chr9: 91120108 | G | A | NA | 0.000742923 |
| | G0000014689 | V9084229 | | | | | |
| SPIN1 | CMX- | CMX- | chr9: 91120393 | A | G | NA | 0.000742923 |
| | G0000014689 | V9084230 | | | | | |
| SPIN1 | CMX- | CMX- | chr9: 91124743 | A | G | NA | 0.000742923 |
| | G0000014689 | V9084231 | | | | | |
| SPIN1 | CMX- | CMX- | chr9: 91126304 | C | T | NA | 0.000742923 |
| | G0000014689 | V9084232 | | | | | |
| SPIN1 | CMX- | CMX- | chr9: 91126736 | G | A | NA | 0.00031089 |
| | G0000014689 | V9084233 | | | | | |
| SPIN1 | CMX- | CMX- | chr9: 91130846 | G | A | NA | 0.000771149 |
| | G0000014689 | V9084234 | | | | | |
| SPIN1 | CMX- | CMX- | chr9: 91131392 | A | G | NA | 0.000934759 |
| | G0000014689 | V9084235 | | | | | |
| SPIN1 | CMX- | CMX- | chr9: 91133854 | T | A | NA | 0.000858194 |
| | G0000014689 | V9084236 | | | | | |
| SPIN1 | CMX- | CMX- | chr9: 91139780 | C | T | NA | 0.000910019 |
| | G0000014689 | V9084237 | | | | | |
| SPIN1 | CMX- | CMX- | chr9: 91146391 | C | T | NA | 0.000484881 |
| | G0000014689 | V9084238 | | | | | |
| SPN | CMX- | CMX- | chr16: 29274955-29761984 | NA | CNV | SPN (1 exon) | NA |
| | G0000023664 | V1697382 | | | loss | | |
| TACC3 | CMX- | CMX- | chr4: 1729556 | G | A | Drastic | NA |
| | G0000006818 | V9084137 | | | | nonsynonymous | |
| TACC3 | CMX- | CMX- | chr4: 1732978 | G | A | Drastic | NA |
| | G0000006818 | V9084138 | | | | nonsynonymous | |
| TLE6 | CMX- | CMX- | chr19: 2946999-3051118 | NA | CNV | TLE6 (2 | NA |
| | G0000026639 | V1806717 | | | loss | exons) | |
| TLE6 | CMX- | CMX- | chr19: 2937389-3057790 | NA | CNV | TLE6 (2 | NA |
| | G0000026639 | V1336365 | | | loss | exons) | |
| ZP3 | CMX- | CMX- | chr7: 76058767 | G | T | Start codon | NA |
| | G0000011947 | V9084143 | | | | gained | |
| NA | NA | CMX- | chr1: 3584692-3585200 | NA | CNV | NA | 0.000363085 |
| | | V2992389 | | | gain | | |
| NA | NA | CMX- | chr1: 33214881-33216355 | NA | CNV | NA | 0.00145087 |
| | | V2992390 | | | loss | | |
| NA | NA | CMX- | chr1: 110252792-110252792 | NA | CNV | NA | 0.00145087 |
| | | V2992391 | | | loss | | |
| NA | NA | CMX- | chr1: 148800056-148802742 | NA | CNV | NA | 0.000363942 |
| | | V2992392 | | | gain | | |
| NA | NA | CMX- | chr2: 86414923-86421116 | NA | CNV | NA | 0.00145087 |
| | | V2992393 | | | loss | | |
| NA | NA | CMX- | chr2: 96237124-96237180 | NA | CNV | NA | 1.33207E−05 |
| | | V2992394 | | | gain | | |
| NA | NA | CMX- | chr2: 215404260-215412550 | NA | CNV | NA | 0.000269506 |
| | | V2992395 | | | loss | | |
| NA | NA | CMX- | chr2: 217210720-217210773 | NA | CNV | NA | 0.00141334 |
| | | V2992396 | | | loss | | |
| NA | NA | CMX- | chr3: 38475943-38476013 | NA | CNV | NA | 0.000263066 |
| | | V2992397 | | | loss | | |
| NA | NA | CMX- | chr3: 150577148-150583696 | NA | CNV | NA | 0.00145087 |
| | | V2992398 | | | loss | | |
| NA | NA | CMX- | chr4: 95892431-95892748 | NA | CNV | NA | 0.000595928 |
| | | V2992399 | | | loss | | |
| NA | NA | CMX- | chr4: 103965296-103966620 | NA | CNV | NA | 9.32084E−05 |
| | | V2992400 | | | gain | | |
| NA | NA | CMX- | chr4: 174691633-174691747 | NA | CNV | NA | 0.001024494 |
| | | V2992401 | | | loss | | |
| NA | NA | CMX- | chr5: 106349950-106350159 | NA | CNV | NA | 0.001666446 |
| | | V2992402 | | | loss | | |
| NA | NA | CMX- | chr5: 179654883-179655477 | NA | CNV | NA | 0.00091471 |
| | | V2992403 | | | loss | | |
| NA | NA | CMX- | chr6: 77073676-77085224 | NA | CNV | NA | 0.00010917 |
| | | V2992404 | | | gain | | |
| NA | NA | CMX- | chr7: 43968000-44039304 | NA | CNV | NA | 0.000860892 |
| | | V2992405 | | | loss | | |
| NA | NA | CMX- | chr7: 69794356-69800088 | NA | CNV | NA | 0.00145087 |
| | | V2992406 | | | loss | | |
| NA | NA | CMX- | chr7: 99464961-99465782 | NA | CNV | NA | 0.00125626 |
| | | V2992407 | | | loss | | |
| NA | NA | CMX- | chr7: 101713977-101923980 | NA | CNV | NA | 0.000860892 |
| | | V2992408 | | | loss | | |
| NA | NA | CMX- | chr8: 12292467-12292467 | NA | CNV | NA | 0.00116959 |
| | | V2992409 | | | gain | | |
| NA | NA | CMX- | chr8: 141723436-141723436 | NA | CNV | NA | 0.001419478 |
| | | V2992410 | | | loss | | |
| NA | NA | CMX- | chr8: 145465005-145465005 | NA | CNV | NA | 0.000488267 |
| | | V2992411 | | | loss | | |
| NA | NA | CMX- | chr9: 119213636-119220054 | NA | NA | NA | 0.001446882 |
| | | V2992412 | | | | | |
| NA | NA | CMX- | chr9: 129199955-129200021 | NA | CNV | NA | 0.00046153 |
| | | V2992413 | | | gain | | |
| NA | NA | CMX- | chr9: 138557819-138563454 | NA | CNV | NA | 0.001446882 |
| | | V2992414 | | | loss | | |
| NA | NA | CMX- | chr10: 13425201-13426135 | NA | CNV | NA | 0.000295719 |
| | | V2992415 | | | loss | | |
| NA | NA | CMX- | chr10: 79352754-79359886 | NA | CNV | NA | 0.00145087 |
| | | V2992416 | | | loss | | |
| NA | NA | CMX- | chr10: 135037958-135044579 | NA | CNV | NA | 0.000983276 |
| | | V2992417 | | | loss | | |
| NA | NA | CMX- | chr11: 2113479-2113533 | NA | CNV | NA | 0.001566125 |
| | | V2992418 | | | loss | | |
| NA | NA | CMX- | chr11: 20521659-20533456 | NA | CNV | NA | 0.001445217 |
| | | V2992419 | | | loss | | |
| NA | NA | CMX- | chr11: 72165348-72167302 | NA | CNV | NA | 0.000366026 |
| | | V2992420 | | | loss | | |
| NA | NA | CMX- | chr12: 110336347-110344141 | NA | CNV | NA | 0.000263066 |
| | | V2992421 | | | loss | | |
| NA | NA | CMX- | chr12: 131580185-131649282 | NA | CNV | NA | 0.000434354 |
| | | V2992422 | | | loss | | |
| NA | NA | CMX- | chr13: 105982985-105988178 | NA | CNV | NA | 0.001566125 |
| | | V2992423 | | | loss | | |
| NA | NA | CMX- | chr14: 104711812-104721574 | NA | CNV | NA | 0.000117224 |
| | | V2992424 | | | loss | | |
| NA | NA | CMX- | chr14: 105554845-105554845 | NA | CNV | NA | 0.00115304 |
| | | V2992425 | | | gain | | |
| NA | NA | CMX- | chr14: 106038187-106038187 | NA | CNV | NA | 0.001388783 |
| | | V2992426 | | | gain | | |
| NA | NA | CMX- | chr15: 72473905-72483708 | NA | CNV | NA | 2.2682E−05 |
| | | V2992427 | | | gain | | |
| NA | NA | CMX- | chr15: 81743011-81748883 | NA | CNV | NA | 0.000934763 |
| | | V2992428 | | | loss | | |
| NA | NA | CMX- | chr15: 97006211-97006211 | NA | CNV | NA | 0.00088514 |
| | | V2992429 | | | loss | | |
| NA | NA | CMX- | chr16: 420035-420035 | NA | CNV | NA | 0.001033484 |
| | | V2992430 | | | loss | | |
| NA | NA | CMX- | chr16: 28297962-28340178 | NA | CNV | NA | 3.83769E−05 |
| | | V2992431 | | | loss | | |
| NA | NA | CMX- | chr16: 28614007-28653740 | NA | CNV | NA | 0.000337601 |
| | | V2992432 | | | loss | | |
| NA | NA | CMX- | chr16: 33772936-33809650 | NA | CNV | NA | 0.001224595 |
| | | V2992433 | | | loss | | |
| NA | NA | CMX- | chr17: 37686892-37687211 | NA | CNV | NA | 0.000263066 |
| | | V2992434 | | | loss | | |
| NA | NA | CMX- | chr17: 70365673-70365673 | NA | CNV | NA | 0.001652185 |
| | | V2992435 | | | loss | | |
| NA | NA | CMX- | chr17: 77418789-77465794 | NA | CNV | NA | 0.000117224 |
| | | V2992436 | | | loss | | |
| NA | NA | CMX- | chr19: 1532671-1549096 | NA | CNV | NA | 0.000934076 |
| | | V2992437 | | | loss | | |
| NA | NA | CMX- | chr19: 18835562-18835562 | NA | CNV | NA | 0.001224595 |
| | | V2992438 | | | loss | | |
| NA | NA | CMX- | chr19: 38480199-38480199 | NA | CNV | NA | 0.000269506 |
| | | V2992439 | | | loss | | |
| NA | NA | CMX- | chr19: 45731785-45732555 | NA | CNV | NA | 0.000229579 |
| | | V2992440 | | | loss | | |
| NA | NA | CMX- | chr19: 53102000-53153808 | NA | CNV | NA | 0.001644428 |
| | | V2992441 | | | gain | | |
| NA | NA | CMX- | chr20: 1500411-1508282 | NA | CNV | NA | 0.000461106 |
| | | V2992442 | | | loss | | |
| NA | NA | CMX- | chr20: 6694925-6696738 | NA | CNV | NA | 0.000934763 |
| | | V2992443 | | | loss | | |
| NA | NA | CMX- | chr20: 61592202-61594834 | NA | CNV | NA | 0.001494022 |
| | | V2992444 | | | loss | | |
| NA | NA | CMX- | chr21: 15355967-15355967 | NA | CNV | NA | 0.001566125 |
| | | V2992445 | | | loss | | |
| NA | NA | CMX- | chr21: 44541166-44547084 | NA | CNV | NA | 0.000257622 |
| | | V2992446 | | | loss | | |
| NA | NA | CMX- | chrX: 100110102-100110152 | NA | CNV | NA | 0.001445217 |
| | | V2992447 | | | loss | | |
| NA | NA | CMX- | chrX: 152934795-152944222 | NA | CNV | NA | 0.000247877 |
| | | V2992448 | | | loss | | |

### Description of Certain Genes

Below are detailed descriptions of some of the fertility genes described in the tables above.

### BARD1

BRCA1-Associated Ring Domain 1 (BARD1) is a gene that forms a heterodimer complex with the BRCA1 gene, and this complex is required for spindle-pole assembly in mitosis, and hence chromosome stability. Mouse embryos carrying homozygous null alleles for BARD1 died between embryonic day 7.5 and embryonic day 8.5 due to severely impaired cell proliferation (McCarthy et al. Molec. Cell. Biol. 23: 5056-5063, 2003).

C6orf221 (KHDC3L)

KH domain containing 3-like, subcortical maternal complex member (KHDC3L). The gene also has the identifier “C6orf221” [Entrez Gene id: 154288, HGNC id: 33699]. KH domains are protein domains that binds to RNA molecules, and KHDC3L is likely involved in genomic imprinting, a phenomenon where genes are expressed in a parental-origin specific manner. KHDC3L gene expression is maximal in germinal vesicle oocytes, tailing off through metaphase II oocytes, and its expression profile is similar to other oocyte-specific genes [Am J Hum Genet. 2011 September 9; 89(3): 451-458]. It is also found within the set of maternal factors that are important for driving egg-to-embryo transition during fertilization [Reproduction. 2010 May; 139(5):809-23]. Mice carrying homozygous null alleles for KHDC3L display a maternal effect defect in embryogenesis with delayed embryonic development and spindle abnormalities resulting in decreased litter sizes for homozygous females. In humans, KHDC3L has been implicated in familial biparental hydatidiform mole, a maternal-effect recessive inherited disorder [Ref: Am J Hum Genet. 2011 Sep. 9; 89(3): 451-458]

### DNMT1

DNA (cytosine-5)-methyltransferase 1 (DNMT1) [Entrez Gene id: 1786, HGNC id: 2976], belongs to a group of enzymes that transfer methyl groups to position 5 of cytosine bases in DNA. While this process, known as DNA methylation, does not alter DNA base composition, it leaves “epigenetic” modifications to DNA molecules that affect the biochemical properties of the DNA region. DNA methylation, mediated by DNMT1, is crucial in determining cell fate during embyogenesis [Genes Dev. 2008 Jun. 15; 22(12):1607-16, Dev Biol. 2002 Jan. 1; 241(1):172-82.]. Mouse embryos carrying homozygous null alleles for DNMT1 survive only to mid-gestation. The expression of the DNMT1 gene is significantly higher in reproductive tissues than other cell types, and is found within the set of maternal factors that are important for driving egg-to-embryo transition during fertilization [Reproduction. 2010 May; 139(5):809-23, BMC Genomics. 2009 Aug. 3; 10:348].

### FMR1

Fragile X Mental Retardation 1 (FMR1) encodes for the RNA-binding protein FMRP that is implicated in the fragile-X syndrome. The inhibition of translation may be a function of FMR1 in vivo, and that failure of mutant FMR1 protein to oligomerize may contribute to the pathophysiologic events leading to fragile X syndrome. Fragile X premutations in female carriers appear to be a risk factor for premature ovarian failure: 16% of the premutation carriers, menopause occurred before the age of 40, compared with none of the full-mutation carriers and 1 (0.4%) of the controls, indicating a significant association between premature menopause and premutation carrier status. [Am. J. Med. Genet. 83: 322-325, 1999]

### FOXO3

Foxhead box O3 (FOXO3) encodes a protein that induces apoptosis in cells, lying within the DNA damage response and repair pathways. FOXO3 knockout female mice exhibit infertility phenotypes, in particular abnormal ovarian follicular function. Mice mutants carrying a homozygous non-synonymous substitution in exon 2 of the FOXO3 gene show loss of fertility of sexual maturity and exhibit premature ovarian failures. [Mammalian Genome 22: 235-248, 2011]

### MUC4

MUC4 belongs to a family of high-molecular-weight glycoproteins that protect and lubricate the epithelial surface of respiratory, gastrointestinal and reproductive tracts. The extracellular domain can interact with an epidermal growth factor receptor on the cell surface to modulate downstream cell growth signaling by stabilizing and/or enhancing the activity of cell growth receptor complexes [Nature Rev. Cancer. 4(1):45-60, 2004]. MUC4 is expressed in the endometrial epithelium and is associated with endometriosis development and endometriosis-related infertility such as embryo implantation [BMC Med. 2011 9:19, 2011].

### NLRP11

NLR family, pyrin domain containing 11 (NLRP11) encodes a leucine-rich protein belonging to a large family of proteins likely involved in inflammation [Nature Rev. Molec. Cell Biol. 4: 95-104, 2003], and is expressed in the ovary, testes and pre-implantation embryos [BMC Evol Biol. 2009 Aug. 14; 9:202. doi: 10.1186/1471-2148-9-202.]. NLRP11 gene expression shows specificity to reproductive tissues.

### NLRP14

NLR family, pyrin domain containing 14 (NLRP14) encodes a leucine-rich protein belonging to a large family of proteins likely involved in inflammation [Nature Rev. Molec. Cell Biol. 4: 95-104, 2003], and is expressed in the ovary, testes and pre-implantation embryos [BMC Evol Biol. 2009 Aug. 14; 9:202. doi: 10.1186/1471-2148-9-202.]. NPRL14 is also found within the set of maternal factors that are important for driving egg-to-embryo transition during fertilization [Reproduction. 2010 May; 139(5):809-23, BMC Genomics. 2009 Aug. 3; 10:348].

### NLRP5

NLRP5 or MATER (Maternal antigen the embryos require), the protein encoded by the Nlrp5 gene, is another highly abundant oocyte protein that is essential in mouse for embryonic development beyond the two-cell stage. MATER was originally identified as an oocyte-specific antigen in a mouse model of autoimmune premature ovarian failure (Tong et al., 25 Endocrinology, 140:3720-3726, 1999). MATER demonstrates a similar expression and subcellular expression profile to PADI6. Like Padi6-null animals, Nlrp5-null females exhibit normal oogenesis, ovarian development, oocyte maturation, ovulation and fertilization. However, embryos derived from Nlrp5-null females undergo a developmental block at the two-cell stage and fail to exhibit normal embryonic genome activation (Tong et al., Nat Genet 26:267-268, 2000; and Tong et al. Mamm Genome 11:281-287, 2000b).

### NLRP8

NLR family, pyrin domain containing 8 (NLRP8) encodes a leucine-rich protein belonging to a large family of proteins likely involved in inflammation [Nature Rev. Molec. Cell Biol. 4: 95-104, 2003], and is expressed in the ovary, testes and pre-implantation embryos [BMC Evol Biol. 2009 Aug. 14; 9:202. doi: 10.1186/1471-2148-9-202.]. NLRP8 gene expression shows specificity to reproductive tissues.

### NPM2

The gene NPM2[Entrez Gene id: 10361, HGNC id: 7930], or nucleoplasmin 2, is a chaperon that binds to histones, and is involved in sperm chromatin remodeling after oocyte entry [Nucleic Acids Res. 2012 June; 40(11): 4861-4878]. NPM2 has been found in a screen for oocyte-specific genes involved in preimplantation embryonic development [Semin Reprod Med. 2007 July; 25(4):243-51], and is differentially expressed during final oocyte maturation and early embryonic development in humans [Feral Steril. 2007 March; 87(3):677-90]. NPM2 is a maternal effect gene critical for nuclear and nucleolar organization and embryonic development, and is found within the set of maternal factors that are important for driving egg-to-embryo transition during fertilization [Reproduction. 2010 May; 139(5):809-23, BMC Genomics. 2009 Aug. 3; 10:348]. NPM2 is associated with abnormal oocyte morphology and reduced fertility in mice, and female mice homozygous null for NPM2 carry defects in preimplantation embryo development, with abnormalities in oocyte and early embryonic nuclei [Science. 2003 Apr. 25; 300(5619):633-6].

### PADI6

Peptidylarginine Deiminase 6 (PADI6)

Padi6 was originally cloned from a 2D murine egg proteome gel based on its relative abundance, and Padi6 expression in mice appears to be almost entirely limited to the oocyte and pre-implantation embryo (Yurttas et al., 2010). Padi6 is first expressed in primordial oocyte follicles and persists, at the protein level, throughout pre-implantation development to the blastocyst stage (Wright et al., Dev Biol, 256:73-88, 2003). Inactivation of Padi6 leads to female infertility in mice, with the Padi6-null developmental arrest occurring at the two-cell stage (Yurttas et al., 2008).

### PMS2

PMS2 is involved in DNA mismatch repair and involved in fertilization and pre-implantation development. It has been identified by knockout mouse studies as one of many maternal effect genes essential for development [Nature Cell Bio. 4 Suppl, pp.s 41-9].

### SCARB1

Scavenger receptor class B, member 1 (SCARB1) gene encodes a glycoprotein that is a receptor for mediating cholesterol transport. SCARB1-null homozygous female mice were infertile with dysfunctional oocytes [J. Clin. Invest. 108: 1717-1722, 2001], hence, mutations in SCARB1 may affect female fertility by regulating lipoprotein metabolism.

### SPIN1

Spindlin 1 (SPIN1) is a gene abundantly expressed in early embryo development, during the transition from oocyte to pluripotent early-embryo. SPIN1 is phosphorylated in a cell-cycle dependent manner and is associated with the meiotic spindle [Development 124: 493-503, 1997].

### TACC3

Transforming, Acidic Coiled-Coil Containing Protein 3 (TACC3). In mice, TACC3 is abundantly expressed in the cytoplasm of growing oocytes, and is required for microtubule anchoring at the centrosome and for spindle assembly and cell survival (Fu et al., 2010). TACC3 is also found within the set of maternal factors that are important for driving egg-to-embryo transition during fertilization [Reproduction. 2010 May; 139(5):809-23, BMC Genomics. 2009 Aug. 3; 10:348].

### ZP1

Zona pellucid glycoprotein 1 (ZP1) encodes for a protein that is a structural component of the zona pellucida—an extracellular matrix that surrounds the oocyte and early embryo.

### ZP2

Zona pellucid glycoprotein 2 (ZP2) encodes for a protein that is a structural component of the zona pellucida—an extracellular matrix that surrounds the oocyte and early embryo. ZP2 binds to acrosome-reacted sperm and is important in preventing polyspermy [Hum Reprod. 2004 July; 19(7):1580-6.].

### ZP3

Zona pellucid glycoprotein 3 (ZP3) [Entrez Gene id: 7784, HGNC id: 13189], is a structural component of the zona pellucida—an extracellular matrix that surrounds the oocyte and early embryo. It is found within the set of maternal factors that are important for driving egg-to-embryo transition during fertilization [BMC Genomics. 2009 Aug. 3; 10:348]. ZP3 is also expressed in oocytes from early ovarian development, and likely to have a role in the development of primordial follicle before zona pellucida formation [Mol Cell Endocrinol. 2008 Jul. 16; 289(1-2):10-5]. Female mice carrying null alleles for ZP3 exhibit decreased ovary size and weight, abnormal ovarian folliculogenesis and ovulation, ultimately resulting in female infertility.

### ZP4

Zona pellucid glycoprotein 4 (ZP4) encodes for a protein that is a structural component of the zona pellucida—an extracellular matrix that surrounds the oocyte and early embryo. ZP4 stimulates acrosome reaction as part of a signaling pathway that involves Protein Kinase A [Biol Reprod. 2008 November; 79(5):869-77]

DNA (Cytosine-5)-Methyltransferase 1 (DNMT1)

[Entrez Gene id: 1786, HGNC id: 2976], belongs to a group of enzymes that transfer methyl groups to position 5 of cytosine bases in DNA. While this process, known as DNA methylation, does not alter DNA base composition, it leaves “epigenetic” modifications to DNA molecules that affect the biochemical properties of the DNA region. DNA methylation, mediated by DNMT1, is crucial in determining cell fate during embyogenesis [Genes Dev. 2008 Jun. 15; 22(12):1607-16, Dev Biol. 2002 Jan. 1; 241(1):172-82.]. Mouse embryos carrying homozygous null alleles for DNMT1 survive only to mid-gestation. The expression of the DNMT1 gene is significantly higher in reproductive tissues than other cell types, and is found within the set of maternal factors that are important for driving egg-to-embryo transition during fertilization [Reproduction. 2010 May; 139(5):809-23, BMC Genomics. 2009 Aug. 3; 10:348].

The gene NPM2 [Entrez Gene id: 10361, HGNC id: 7930], or nucleoplasmin 2, is a chaperon that binds to histones, and is involved in sperm chromatin remodeling after oocyte entry [Nucleic Acids Res. 2012 June; 40(11): 4861-4878]. NPM2 has been found in a screen for oocyte-specific genes involved in preimplantation embryonic development [Semin Reprod Med. 2007 July; 25(4):243-51], and is differentially expressed during final oocyte maturation and early embryonic development in humans [Feral Steril. 2007 March; 87(3):677-90]. NPM2 is a maternal effect gene critical for nuclear and nucleolar organization and embryonic development, and is found within the set of maternal factors that are important for driving egg-to-embryo transition during fertilization [Reproduction. 2010 May; 139(5):809-23, BMC Genomics. 2009 Aug. 3; 10:348]. NPM2 is associated with abnormal oocyte morphology and reduced fertility in mice, and female mice homozygous null for NPM2 carry defects in preimplantation embryo development, with abnormalities in oocyte and early embryonic nuclei [Science. 2003 Apr. 25; 300(5619):633-6].

Oocyte-Expressed Protein (OOEP)

[Entrez Gene id: 441161, HGNC id: 21382], also goes by the identifiers KHDC2, FLOPED, HOEP19 and C6orf156. OOEP is found within the set of maternal factors that are important for driving egg-to-embryo transition during fertilization [Reproduction. 2010 May; 139(5):809-23]. OOEP is expressed in ovaries, but not detectable in 11 other cell types including male testes. Within the ovary, its expression is restricted to growing oocytes. The OOEP protein product sublocalizes to the subcortex of eggs and preimplantation embryos. OOEP homozygous null female mice have seemingly normal ovarian physiology and produced viable eggs that can be fertilized, however, these embryos do not progress beyond cleavage stage development and hence these female mice are sterile. It is believed that a functioning OOEP is a pre-requisite for pre-implantation mouse development [Dev Cell. 2008 September; 15(3): 416-425.].

Factor Located in Oocytes Permitting Embryonic Development (FLOPED/OOEP)

The subcortical maternal complex (SCMC) is a poorly characterized murine oocyte structure to which several maternal effect gene products localize (Li et al. Dev Cell 15:416-425, 2008). PADI6, MATER, FILIA, TLE6, and FLOPED have been shown to localize to this complex (Li et al. Dev Cell 15:416-425, 2008; Yurttas et al. Development 135:2627-2636, 2008). This complex is not present in the absence of Floped and Nlrp5, and similar to embryos resulting from Nlrp5-depleted oocytes, embryos resulting from Floped-null oocytes do not progress past the two cell stage of mouse development (Li et al., 2008). FLOPED is a small (19 kD) RNA binding protein that has also been characterized under the name of MOEP19 (Herr et al., Dev Biol 314:300-316, 2008).

Zona Pellucid Glycoprotein 3 (ZP3)

[Entrez Gene id: 7784, HGNC id: 13189], is a structural component of the zona pellucida—an extracellular matrix that surrounds the oocyte and early embryo. It is found within the set of maternal factors that are important for driving egg-to-embryo transition during fertilization [BMC Genomics. 2009 Aug. 3; 10:348]. ZP3 is also expressed in oocytes from early ovarian development, and likely to have a role in the development of primordial follicle before zona pellucida formation [Mol Cell Endocrinol. 2008 Jul. 16; 289(1-2):10-5]. Female mice carrying null alleles for ZP3 exhibit decreased ovary size and weight, abnormal ovarian folliculogenesis and ovulation, ultimately resulting in female infertility.

FIGLA (Factor in Germline Alpha)

[Entrez Gene id: 344018, HGNC id:], also goes by the gene identifiers POF6, BHLHC8, and FIGALPHA. This gene is a basic helix-loop-helix transcription factor that acts as an activator of oocyte genes. FIGLA is expressed in all ovarian follicular stages and in mature oocytes, and is required for normal folliculogenesis. FIGLA expression is also believed to repress genes expressed normal in male testes, and hence sustains the female phenotype by activating female and repressing male germ cell genetic hierarchies in growing oocytes during postnatal ovarian development [Mol Cell Biol. 2010 July; 30(14]. Female mice with FIGLA mutations result in decreased oocytes numbers and abnormal ovarian folliculogenesis. Heterozygous mutations in FIGLA has been implicated in women with premature ovarian failure [Am J Hum Genet. 2008 June; 82(6):1342-8.].

Peptidylarginine Deiminase 6 (PADI6)

Padi6 was originally cloned from a 2D murine egg proteome gel based on its relative abundance, and Padi6 expression in mice appears to be almost entirely limited to the oocyte and pre-implantation embryo (Yurttas et al., 2010). Padi6 is first expressed in primordial oocyte follicles and persists, at the protein level, throughout pre-implantation development to the blastocyst stage (Wright et al., Dev Biol, 256:73-88, 2003). Inactivation of Padi6 leads to female infertility in mice, with the Padi6-null developmental arrest occurring at the two-cell stage (Yurttas et al., 2008).

Maternal Antigen the Embryos Require (MATER/NLRP5)

MATER, the protein encoded by the Nlrp5 gene, is another highly abundant oocyte protein that is essential in mouse for embryonic development beyond the two-cell stage. MATER was originally identified as an oocyte-specific antigen in a mouse model of autoimmune premature ovarian failure (Tong et al., Endocrinology, 140:3720-3726, 1999). MATER demonstrates a similar expression and subcellular expression profile to PADI6. Like Padi6-null animals, Nlrp5-null females exhibit normal oogenesis, ovarian development, oocyte maturation, ovulation and fertilization. However, embryos derived from Nlrp5-null females undergo a developmental block at the two-cell stage and fail to exhibit normal embryonic genome activation (Tong et al., Nat Genet 26:267-268, 2000; and Tong et al. Mamm Genome 11:281-287, 2000b).

KH Domain Containing 3-Like, Subcortical Maternal Complex Member (FILIA/KHDC3L)

FILIA is another small RNA-binding domain containing maternally inherited murine protein. FILIA was identified and named for its interaction with MATER (Ohsugi et al. Development 135:259-269, 2008). Like other components of the SCMC, maternal inheritance of the Khdc3 gene product is required for early embryonic development. In mice, loss of Khdc3 results in a developmental arrest of varying severity with a high incidence of aneuploidy due, in part, to improper chromosome alignment during early cleavage divisions (Li et al., 2008). Khdc3 depletion also results in aneuploidy, due to spindle checkpoint assembly (SAC) inactivation, abnormal spindle assembly, and chromosome misalignment (Zheng et al. Proc Natl Acad Sci USA 106:7473-7478, 2009).

Basonuclin (BNC1)

Basonuclin is a zinc finger transcription factor that has been studied in mice. It is found expressed in keratinocytes and germ cells (male and female) and regulates rRNA (via polymerase I) and mRNA (via polymerase II) synthesis (Iuchi and Green, 1999; Wang et al., 2006). Depending on the amount by which expression is reduced in oocytes, embryos may not develop beyond the 8-cell stage. In Bsn1 depleted mice, a normal number of oocytes are ovulated even though oocyte development is perturbed, but many of these oocytes cannot go on to yield viable offspring (Ma et al., 2006).

Zygote Arrest 1 (ZAR1)

Zar1 is an oocyte-specific maternal effect gene that is known to function at the oocyte to embryo transition in mice. High levels of Zar1 expression are observed in the cytoplasm of murine oocytes, and homozygous-null females are infertile: growing oocytes from Zar1-null females do not progress past the two-cell stage.

Cytosolic Phospholipase A2γ (PLA2G4C)

Under normal conditions, cPLA2γ, the protein product of the murine PLA2G4C ortholog, expression is restricted to oocytes and early embryos in mice. At the subcellular level, cPLA2γ mainly localizes to the cortical regions, nucleoplasm, and multivesicular aggregates of oocytes. It is also worth noting that while cPLA2γ expression does appear to be mainly limited to oocytes and pre-implantation embryos in healthy mice, expression is considerably up-regulated within the intestinal epithelium of mice infected with Trichinella spiralis. This suggests that cPLA2γ may also play a role in the inflammatory response. The human PLA2G4C differs in that rather than being abundantly expressed in the ovary, it is abundantly expressed in the heart and skeletal muscle. Also, the human protein contains a lipase consensus sequence but lacks a calcium-binding domain found in other PLA2 enzymes. Accordingly, another cytosolic phospholipase may be more relevant for human fertility.

Transforming, Acidic Coiled-Coil Containing Protein 3 (TACC3)

In mice, TACC3 is abundantly expressed in the cytoplasm of growing oocytes, and is required for microtubule anchoring at the centrosome and for spindle assembly and cell survival (Fu et al., 2010). In certain embodiments, the gene is a gene that is expressed in an oocyte. Exemplary genes include CTCF, ZFP57, POU5F1, SEBOX, and HDAC1.

In other embodiments, the gene is a gene that is involved in DNA repair pathways, including but not limited to, MLH1, PMS1 and PMS2. In other embodiments, the gene is BRCA1 or BRCA2.

In other embodiments, the biomarker is a gene product (e.g., RNA or protein) of an infertility-associated gene. In particular embodiments, the gene product is a gene product of a maternal effect gene. In other embodiments, the gene product is a product of a gene from Table 1. In certain embodiments, the gene product is a product of a gene that is expressed in an oocyte, such as a product of CTCF, ZFP57, POU5F1, SEBOX, and HDAC1. In other embodiments, the gene product is a product of a gene that is involved in DNA repair pathways, such as a product of MLH1, PMS1, or PMS2. In other embodiments, gene product is a product of BRCA1 or BRCA2.

In other embodiments, the biomarker may be an epigenetic factor, such as methylation patterns (e.g., hypermethylation of CpG islands), genomic localization or post-translational modification of histone proteins, or general post-translational modification of proteins such as acetylation, ubiquitination, phosphorylation, or others.

In other embodiments, methods of the invention analyze infertility-associated biomarkers in order to assess the risk infertility.

In certain embodiments, the biomarker is a genetic region, gene, or RNA/protein product of a gene associated with the one carbon metabolism pathway and other pathways that effect methylation of cellular macromolecules. Exemplary genes and products of those genes are described below.

Methylenetetrahydrofolate Reductase (MTHFR)

In particular embodiments a mutation (677C>T) in the MTHFR gene is associated with infertility. The enzyme 5,10-methylenetetrahydrofolate reductase regulates folate activity (Pavlik et al., Fertility and Sterility 95(7): 2257-2262, 2011). The 677TT genotype is known in the art to be associated with 60% reduced enzyme activity, inefficient folate metabolism, decreased blood folate, elevated plasma homocysteine levels, and reduced methylation capacity. Pavlik et al. (2011) investigated the effect of the MTHFR 677C>T on serum anti-Mullerian hormone (AMH) concentrations and on the numbers of oocytes retrieved (NOR) following controlled ovarian hyperstimulation (COH). Two hundred and seventy women undergoing COH for IVF were analyzed, and their AMH levels were determined from blood samples collected after 10 days of GnRH superagonist treatment and before COH. Average AMH levels of TT carriers were significantly higher than those of homozygous CC or heterozygous CT individuals. AMH serum concentrations correlated significantly with the NOR in all individuals studied. The study concluded that the MTHFR 677TT genotype is associated with higher serum AMH concentrations but paradoxically has a negative effect on NOR after COH. It was proposed that follicle maturation might be retarded in MTHFR 677TT individuals, which could subsequently lead to a higher proportion of initially recruited follicles that produce AMH, but fail to progress towards cyclic recruitment. The tissue gene expression patterns of MTHFR do not show any bias towards oocyte expression. Analyzing a sample for this mutation or other mutations (Table 1) in the MTHFR gene or abnormal gene expression of products of the MTHFR gene allows one to assess a risk of infertility.

Jeddi-Tehrani et al. (American Journal of Reproductive Immunology 66(2):149-156, 2011) investigated the effect of the MTHFR 677TT genotype on Recurrant Pregnancy Loss (RPL). One hundred women below 35 years of age with two successive pregnancy losses and one hundred healthy women with at least two normal pregnancies were used to assess the frequency of five candidate genetic risk factors for RPL-MTHFR 677C>T, MTHFR 1298A>C, PAII-675 4G/5G (Plasminogen Activator Inhibitor-1 promoter region), BF-455G/A (Beta Fibrinogen promoter region), and ITGB3 1565T/C (Integrin Beta 3). The frequencies of the polymorphisms were calculated and compared between case and control groups. Both the MTHFR polymorphisms (677C>T and 1298 A>C) and the BF-455G/A polymorphism were found to be positively and ITGB3 1565T/C polymorphism was found to be negatively associated with RPL. Homozygosity but not heterozygosity for the PAI-1-6754G/5G polymorphism was significantly higher in patients with RPL than in the control group. The presence of both mutations of MTHFR genes highly increased the risk of RPL. Analyzing a sample for these mutation and other mutations (Table 1) in the MTHFR gene or abnormal gene expression of products of the MTHFR gene allows one to assess a risk of infertility.

Catechol-O-Methyltransferase (COMT)

In particular embodiments a mutation (472G>A) in the COMT gene is associated with infertility. Catechol-O-methyltransferase is known in the art to be one of several enzymes that inactivates catecholamine neurotransmitters by transferring a methyl group from SAM (S-adenosyl methionine) to the catecholamine. The AA gene variant is known to alter the enzyme's thermostability and reduces its activity 3 to 4 fold (Schmidt et al., Epidemiology 22(4): 476-485, 2011). Salih et al. (Fertility and Sterility 89(5, Supplement 1): 1414-1421, 2008) investigated the regulation of COMT expression in granulosa cells and assessed the effects of 2-ME2 (COMT product) and COMT inhibitors on DNA proliferation and steroidogenesis in JC410 porcine and HGL5 human granulosa cell lines in in vitro experiments. They further assessed the regulation of COMT expression by DHT (Dihydrotestosterone), insulin, and ATRA (all-trans retinoic acid). They concluded that COMT expression in granulosa cells was up-regulated by insulin, DHT, and ATRA. Further, 2-ME2 decreased, and COMT inhibition increased granulosa cell proliferation and steroidogenesis. It was hypothesized that COMT overexpression with subsequent increased level of 2-ME2 may lead to ovulatory dysfunction. Analyzing a sample for this mutation in the COMT gene or abnormal gene expression of products of the COMT gene allows one to assess a risk of infertility.

Methionine Synthase Reductase (MTRR)

In particular embodiments a mutation (A66G) in the Methionine Synthase Reductase (MTRR) gene is associated with infertility. MTRR is required for the proper function of the enzyme Methionine Synthase (MTR). MTR converts homocysteine to methionine, and MTRR activates MTR, thereby regulating levels of homocysteine and methionine. The maternal variant A66G has been associated with early developmental disorders such as Down's syndrome (Pozzi et al., 2009) and Spina Bifida (Doolin et al., American journal of human genetics 71(5): 1222-1226, 2002). Analyzing a sample for this mutation in the MTRR gene or abnormal gene expression of products of the MTRR gene allows one to assess the risk of infertility.

Betaine-Homocysteine S-Methyltransferase (BHMT)

In particular embodiments a mutation (G716A) in the BHMT gene is associated with infertility. Betaine-Homocysteine S-Methyltransferase (BHMT), along with MTRR, assists in the Folate/B-12 dependent and choline/betaine-dependent conversions of homocysteine to methionine. High homocysteine levels have been linked to female infertility (Berker et al., Human Reproduction 24(9): 2293-2302, 2009). Benkhalifa et al. (2010) discuss that controlled ovarian hyperstimulation (COH) affects homocysteine concentration in follicular fluid. Using germinal vesicle oocytes from patients involved in IVF procedures, the study concludes that the human oocyte is able to regulate its homocysteine level via remethylation using MTR and BHMT, but not CBS (Cystathione Beta Synthase). They further emphasize that this may regulate the risk of imprinting problems during IVF procedures. Analyzing a sample for this mutation in the BHMT gene or abnormal gene expression of products of the BHMT gene allows one to assess a risk of infertility.

Ikeda et al. (Journal of Experimental Zoology Part A: Ecological Genetics and Physiology 313A(3): 129-136, 2010) examined the expression patterns of all methylation pathway enzymes in bovine oocytes and preimplantation embryos. Bovine oocytes were demonstrated to have the mRNA of MAT1A (Methionine adenosyltransferase), MAT2A, MAT2B, AHCY (S-adenosylhomocysteine hydrolase), MTR, BHMT, SHMT1 (Serine hydroxymethyltransferase), SHMT2, and MTHFR. All these transcripts were consistently expressed through all the developmental stages, except MAT1A, which was not detected from the 8-cell stage onward, and BHMT, which was not detected in the 8-cell stage. Furthermore, the effect of exogenous homocysteine on preimplantation development of bovine embryos was investigated in vitro. High concentrations of homocysteine induced hypermethylation of genomic DNA as well as developmental retardation in bovine embryos. Analyzing a sample for these irregular methylation patterns allows one to assess a risk of infertility.

Folate Receptor 2 (FOLR2)

In particular embodiments a mutation (rs2298444) in the FOLR2 gene is associated with infertility. Folate Receptor 2 helps transport folate (and folate derivatives) into cells. Elnakat and Ratnam (Frontiers in bioscience: a journal and virtual library 11: 506-519, 2006) implicate FOLR2, along with FOLR1, in ovarian and endometrial cancers. Analyzing sample mutations in the FOLR2 or FOLR1 genes or abnormal gene expression of products of the FOLR2 or FOLR1 genes allows one to assess a risk of infertility.

Transcobalamin 2 (TCN2)

In particular embodiments a mutation (C776G) in the TCN2 gene is associated with infertility. Transcobalamin 2 facilitates transport of cobalamin (Vitamin B12) into cells. Stanislawska-Sachadyn et al. (Eur J ClinNutr 64(11): 1338-1343, 2010) assessed the relationship between TCN2 776C>G polymorphism and both serum B12 and total homocysteine (tHcy) levels. Genotypes from 613 men from Northern Ireland were used to show that the TCN2 776CC genotype was associated with lower serum B12 concentrations when compared to the 776CG and 776GG genotypes. Furthermore, vitamin B12 status was shown to influence the relationship between TCN2 776C>G genotype and tHcy concentrations. The TCN2 776C>G polymorphism may contribute to the risk of pathologies associated with low B12 and high total homocysteine phenotype. Analyzing a sample for this mutation in the TCN2 gene or abnormal gene expression of products of the TCN2 gene allows one to assess a risk of infertility.

Cystathionine-Beta-Synthase (CBS)

In particular embodiments a mutation (rs234715) in the CBS gene is associated with infertility. With vitamin B6 as a cofactor, the Cystathionine-Beta-Synthase (CBS) enzyme catalyzes a reaction that permanently removes homocysteine from the methionine pathway by diverting it to the transsulfuration pathway. CBS gene mutations associated with decreased CBS activity also lead to elevated plasma homocysteine levels. Guzman et al. (2006) demonstrate that Cbs knockout mice are infertile. They further explain that Cbs-null female infertility is a consequence of uterine failure, which is a consequence of hyperhomocysteinemia or other factor(s) in the uterine environment. Analyzing a sample for this mutation in the CBS gene or abnormal gene expression of products of the CBS gene allows one to assess a risk of infertility.

In certain embodiments, the biomarker is a genetic region that has been previously associated with female infertility. A SNP association study by targeted re-sequencing was performed to search for new genetic variants associated with female infertility. Such methods have been successful in identifying significant variants associated in a wide range of diseases Rehman et al., 2010; Walsh et al., 2010). Briefly, a SNP association study is performed by collecting SNPs in genetic regions of interest in a number of samples and controls and then testing each of the SNPs that showed significant frequency differences between cases and controls. Significant frequency differences between cases and controls indicate that the SNP is associated with the condition of interest.

In certain embodiments, genetic loci to be investigated in a mouse model are derived from a cluster analysis, discussed below. As stated above, other methods to determine a genetic region of interest can be employed, i.e., human test results or findings published in literature.

### Cluster Analysis

In addition to using infertility biomarkers identified above, methods of the invention further utilize the existing infertility knowledgebase to identify commonalities between known infertility genes and genes having no prior association with infertility. By identifying commonalities between infertility genes and genes having no prior association with infertility, one is able to expand the list of potential genes associated with infertility and guide understanding as to what gene functions and changes are causally-linked to infertility. For example, genes having commonalities with known infertility genes can be identified as potential infertility biomarkers, and used in phenotypic studies (such those performed in mice) related to infertility, thereby expanding the breadth infertility knowledgebase.

In order to determine commonalities between infertility genes and genes without prior associated with infertility, methods of the invention utilize cluster analysis techniques. Generally, a cluster analysis involves grouping a set of objects in such a way that certain objects are clustered in one group are more similar to each other than objects in another group or cluster. Methods of the invention cluster known infertility genes with genes not associated with infertility based on features such as gene expression, phenotype, and genetic pathways. From the cluster analysis, one can identify genes without prior association with infertility that exhibit features with a high degree of similarity (relatedness) to infertility genes. Those genes exhibiting a high degree of similarity (as shown through the cluster analysis) can be identified as a potential infertility biomarker.

The following describes a clustering method used to identify a potential infertility biomarker in accordance with methods of the invention. The method is typically a computer-implemented method, e.g. utilizes a computer system that includes a processor and a computer readable storage medium. The processor of the computer system executes instructions obtained from the computer-readable storage device to perform the cluster analysis.

In accordance with to certain aspects, the method involves obtaining a gene data set that includes both known infertility genes and genes having no prior association with infertility. The genes forming the cluster data set (those associated with infertility and those not known to be associated with infertility) are typically mammalian genes. The mammalian genes may correspond to mouse genes, human, genes, or a combination thereof. A cluster analysis is then performed on the gene data set to determine a relationship between the one or more genes not associated with infertility and the known infertility genes. If a gene not associated with infertility is shown to cluster with a known infertility gene, the method provides for identifying that gene as a potential infertility biomarker. If the gene not associated with infertility does not cluster with a known infertility gene, then that gene is less likely to be causally linked to infertility in the same/similar manner as that known infertility gene.

Methods of the invention assess several features (or parameters) of genes in order to determine commonalities and thus cluster genes not associated with infertility with known infertility genes based on the commonalities. In certain embodiments, those features include gene expression, phenotypes, gene pathways, and a combination thereof. One or more of those features can contribute to a gene's position in the clustering.

Feature data (such as gene expression, phenotype, gene pathway, etc.) is obtained for both known infertility genes and genes not known to be associated with infertility. The feature and gene data is compiled to form a matrix that will be used to exhibit the cluster analysis. For example, the feature data is pre-processed to express each domain as a row and each feature as a column (or vice versa). For domains with continuous values such as gene expression, the features are the individual tissues where gene expression was measured, and each value in the matrix (Xij) represents the expression of gene i in tissue j. For domains with categorical values such as phenotypes, the features are the individual phenotypes, and each value in the matrix (Xij) is a binary indicator representing whether gene i is associated with phenotype j. All of the domain specific matrices are then combined column-wise. A distance metric is then applied to each pair of rows and each pair of columns in the matrix. In certain embodiments, the distance metric is ‘Distance=1-correlation’. However, it is understood that other standard distance metrics could be used (e.g. Euclidean).

Standard hierarchical clustering was then used to cluster the rows and columns of the matrix in order to determine feature commonalities between known infertility genes and other genes. Various hierarchical clustering techniques are known in the art, and can be applied to methods of the invention for clustering infertility genes with genes not associated with infertility. Hierarchical clustering techniques are described in, for example, Sturn, Alexander, John Quackenbush, and Zlatko Trajanoski. “Genesis: cluster analysis of microarray data.” Bioinformatics 18.1 (2002): 207-208; Yeung, Ka Yee, and Walter L. Ruzzo. “Principal component analysis for clustering gene expression data.” Bioinformatics 17.9 (2001): 763-774; Eisen, Michael B., et al. “Cluster analysis and display of genome-wide expression patterns.” Proceedings of the National Academy of Sciences 95.25 (1998): 14863-14868. Generally, clustering involves comparing features of one or more genes not associated with features of one or more known infertility, and categorizing the genes into one or more feature groups based on the comparison. After the comparison, the cluster analysis may further involve assigning a value to the categorized genes based on a degree of relatedness. For example, genes clustered together having highly similar or the same features may be assigned a high value (e.g. positive integer). The degree of relatedness may be highlighted on the resulting cluster matrix via colors, e.g. high degree of commonality being shown in red and low degree of commonality being shown in blue.

After a hierarchical clustering technique is applied to the gene/feature data, the gene clusters are displayed against certain feature categories (e.g. phenotype/gene expression ‘category’), which are then clustered to reflect commonality. For example, phenotypes of female reproduction are grouped together in one cluster, and phenotypes of embryo patterning, morphology and growth are grouped in a separate cluster, etc. The degree of relatedness or commonality between clustered genes (as determined by the cluster analysis) can then be highlighted on the resulting cluster matrix. For example, red may be used to indicate that the gene is associated with one very specific phenotype and/or is expressed at high levels in the associated tissue/physiological system indicated on the opposite axis; whereas blue may be used to indicate that the gene is associated with a number of different and varied phenotypes and/or is expressed at low levels in the associated tissue.

By clustering genes into feature specific groups and color-coding genes with high degree of relatedness, the resulting cluster matrix of the invention advantageously allows for visualization of groups of genes that are strongly associated with phenotypes relating to particular tissues or physiological systems (i.e. clusters of interest). Thus, cluster matrices of the invention allow one to quickly identify genes without prior association with infertility as potential infertility biomarkers based on their shown association (cluster) with known infertility biomarkers. This clustering and identification of potential infertility biomarkers is done independently from and without correlating a gene's proximity with other genes within or location on the Fertilome (genomic region associated with infertility). As a result, clustering provides an additional method of identifying infertility genes of interest that can be used to complement and in addition to other techniques for identifying infertility genes of interest.

The following describes a specific example of using the above described cluster analysis to correlate genes not known to be associated with infertility and a known infertility gene.

Activin receptor 2b (ACVR2B) is a significant copy number variation identified in a cohort of patients with infertility (i.e. copy number variation in this gene was identified as being significantly associated with an infertile phenotype in humans). Activin receptor 2B is the receptor bound by Activin, a protein previously known in the art to be involved in both human and mouse reproduction and embryonic development. Activin/Nodal signaling regulates pluripotency and several aspects of patterning during early embryogenesis. Together with Inhibin and Follistatin, Activin is also involved in the complex feedback loops that selectively regulate FSH secretion.

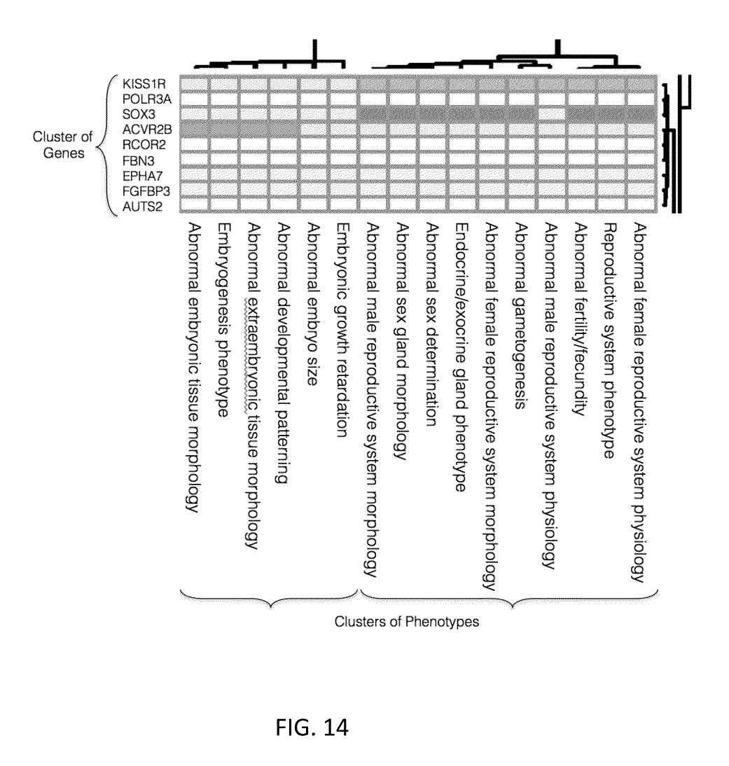

A cluster analysis was performed that compared those features of ACVR2B and features of a plurality of genes not known to be associated with infertility. Based on the cluster analysis, several of the plurality of genes were determined to cluster with the ACVR2B gene due to a commonality between functional and phenotypic features. The genes clustered with the ACVR2B gene were thus identified as potential infertility biomarkers. FIG. 14 illustrates the results of a cluster analysis with ACVR2B.

Cluster analysis as applicable to mouse modeling is further described in more detail below. As discussed, clustering analysis provides more functional information with regards to infertility suspected genetic loci and biomarkers by putting genetic loci in clusters according to attributes including phenotype and tissue expression level/pattern. Results of the cluster analysis reveal genetic loci that have a newly predicted association with the other loci in the cluster. Prior, there may have been no existing indication of a direct functional link in the literature. Thus, cluster nalysis may be used to highlight new genetic loci for further phenotypic study in mouse models, and can create knowledge of how particular genetic loci cluster together to provide understanding of how mutation(s) in the gene(s) of interest might bring about the molecular, cellular and physiological changes sufficient to affect particular aspects of infertility.

Attributes such as expression, phenotype, or knowledge of gene pathways or a combination of any of these can contribute to a gene's position in the clustering. Data from one, two, or any combination of these parameters are pre-processed to express each domain as a matrix with genetic loci in rows and features in columns. For domains with continuous values such as gene expression, the features are the individual tissues where gene expression was measured, and each value in the matrix (Xij) represents the expression of gene i in tissue j. For domains with categorical values such as phenotypes, the features are the individual phenotypes, and each value in the matrix (Xij) is a binary indicator representing whether gene i is associated with phenotype j. All of the domain specific matrices are then combined column-wise. A distance metric is then applied to each pair of rows and each pair of columns in the matrix (for example,′Distance=1-correlation′), but other standard distance metrics could be used (e.g., Euclidean). Standard hierarchical clustering can then used to cluster the rows and columns of the matrix.

The gene clusters are displayed against an attribute such as phenotype/gene expression ‘category’, which is in turn ‘clustered’ to reflect commonality. For example, phenotypes of female reproduction are grouped together in one cluster. Phenotypes of embryo patterning, morphology and growth are grouped in a separate cluster, etc. Measurement can be indicated by a color scale, for example, where red may indicate that the gene is associated with one very specific phenotype and/or is expressed at high levels in the associated tissue/physiological system indicated on the opposite axis; whereas blue indicates the gene is associated with a number of different and varied phenotypes and/or is expressed at low levels in the associated tissue. Therefore correlations can be visualized of groups of genetic loci that are strongly associated with phenotypes relating to particular tissues or physiological systems. The clustering is done independent of any information regarding the physical proximity of these genetic elements on the chromosome. The method of clustering allows both a narrow- and wide-scale view of groups of genetic loci and their association with [a] particular phenotype(s), highlighting groups of genetic loci likely to function in a similar way and in some cases even together, to regulate particular aspects of infertility.

According to certain embodiments, a cluster analysis is created by first combining a database is compiled that includes features attributed to each nucleotide of the human genome including functional annotation such as gene boundaries, exons, splice sites, areas of putative non-coding RNAs and other elements such as promoters or CpG islands and features associated with those regions such as tissue-specific transcriptional expression from multiple mammalian systems including mouse and human, transgenic mouse strain phenotypes, mutations in genetic loci or genetic regions that have been associated with different human diseases, the relationship of particular genetic loci to particular molecular or cellular pathways, gene ontology, protein-protein interactions, and mutations that have been observed. Some of the data is from public sources (e.g., mouse phenotypes) and some data is from research studies (e.g., non-public data related to mouse phenotypes and non-coding areas of interest or coding region mutations observed in patients with infertility).

After the database is assembled, a meta-analysis on the gene regions is performed in the following way. First, the data is pre-processing to express each domain as a matrix with genetic loci in rows and features in columns. For domains with continuous values such as gene expression, the features are the individual tissues where gene expression was measured, and each value in the matrix (Xij) represents the expression of gene i in tissue j. For domains with categorical values such as phenotypes, the features are the individual phenotypes, and each value in the matrix (Xij) is a binary indicator representing whether gene i is associated with phenotype j. Each domain matrix has R rows and Ck columns

Each domain matrix is then scaled so that each gene has mean 0 and standard deviation 1. All of the domain specific matrices are then combined column-wise, giving a matrix with R rows and ΣCk columns.

A distance metric is then applied to each pair of rows and each pair of columns in the matrix. Here, the weighted correlation value is the Pearson correlation with higher weights applied to specific features (columns). Since interest is in infertility driven clustering, infertility/reproductive associated phenotypes and tissues are given higher weights in the correlation value and hence in the distance calculation. Alternate weights could be used to emphasize other aspects of the gene information. The resulting distance value is 0 for genetic loci with identical annotation, and 1 for completely uncorrelated annotation.

Standard hierarchical clustering is then used to cluster the rows and columns of the matrix. An intensity-based coloring is used on the values in the matrix with red indicating a higher positive signal. The gene-wise distances and the associated clustering have several uses.

For example, starting from known infertility associated genetic loci in one mammalian species such as mouse, one can identify novel infertility associated genetic loci in the same species or another mammalian species that contains an orthologous gene. As an example, starting with the known human infertility gene NLRP5, Table 8 lists the most similar (smallest distance) genes to NLRP5. Most of the genes on the list have already been identified based on published studies as having an association with infertility (a validation of the approach), but several have not (e.g., ATAD2B, NR2E1). In this example, ATAD2B, NR2E1 are good candidates for studies/analysis to confirm their infertility association.

For example, starting with a partially characterized gene, impute likely phenotypes/pathways based on co-clustered genetic loci. As an example, the gene CHST8 has incomplete annotation regarding its role in human biological pathways and diseases, including infertility. Table 9 shows the genes most similar in function to CHST8 based on the clustering method. The fertility-associated genes FSHB and LHB are characterized as being similar to, or having similar function to CHST8, and are both well characterized independently. Both encode binding proteins for hormones important in female fertility. In this example, CHST8 is therefore a good candidate for studies/analysis to reveal how it is associated with infertility, for example through the disruption of the CHST8 gene in a transgenic mouse model.

For example, identify clusters of related infertility-associated genetic loci that may be used for the development of an infertility assay in humans [Pittman, Jennifer, et al. “Integrated modeling of clinical and gene expression information for personalized prediction of disease outcomes.” Proceedings of the National Academy of Sciences of the United States of America 101.22 (2004): 8431-8436]. FIG. 14 shows a cluster of genes, each with their own particular gene annotation, curated from knowledge in the literature such as but not limited to, tissue-specific gene expression level, association of the gene or genetic region with (a) particular phenotype/s, association of the gene or genetic region with particular cellular pathway, and protein-protein interactions. Membership in a cluster is based on a genetic region demonstrating similar attributes in these domains, and on the division of the clustering tree into sections depending on the degree of functional relatedness of genetic loci within particular clusters, calculated by the attributes listed. In an alternative embodiment a method such as k-means could be used. The present methodology determines that each cluster of genetic loci may be involved with a separate aspect of fertility (e.g., oocyte development, hormone signaling, embryo implantation). These clusters could then serve as the basis of assays to assess human infertility, or as candidates for the creation of genetically altered mice to provide a model for infertility, as well as the means to test infertility treatments, such as those provided by, but not limited to, therapeutic drugs. The clusters can also be used empirically, without knowing their association with specific characteristics of infertility, by creating meta-genes. A meta-gene is a weighted combination of a set of genetic loci, and functions as a single predictor of human infertility that integrates effects from multiple similar genetic loci. The use of meta-genes can significantly increase the power of genetic/genomic studies by increasing the predictive strength and reducing the number of hypotheses tested.

TABLE 8

| | | Known | | |
|---|---|---|---|---|
| | | Infertility | | Similarity |
| entrezGeneId | symbol | Association | MouseGeneId | (1-Distance) |
| 126206 | NLRP5 | Y | 23968 | 1 |
| 441161 | OOEP | Y | 67968 | 0.990508 |
| 326340 | ZAR1 | Y | 317755 | 0.954272 |
| 359787 | DPPA3 | Y | 73708 | 0.925278 |
| 54454 | ATAD2B | | 320817 | 0.768295 |
| 8115 | TCL1A | Y | 21432 | 0.729399 |
| 4361 | MRE11A | | 17535 | 0.728909 |
| 4360 | MRC1 | Y | 17533 | 0.727167 |
| 7101 | NR2E1 | | 21907 | 0.719154 |
| 23633 | KPNA6 | | 16650 | 0.712841 |
| 2827 | GPR3 | Y | 14748 | 0.709265 |
| 7783 | ZP2 | Y | 22787 | 0.709177 |
| 200424 | TET3 | | 194388 | 0.707759 |
| 127343 | DMBX1 | Y | 140477 | 0.704141 |
| 10361 | NPM2 | Y | 328440 | 0.700169 |
| 7784 | ZP3 | Y | 22788 | 0.696949 |
| 9210 | BMP15 | Y | 12155 | 0.688272 |
| 22917 | ZP1 | Y | 22786 | 0.688209 |
| 54014 | BRWD1 | Y | 93871 | 0.681323 |
| 344018 | FIGLA | Y | 26910 | 0.674247 |
| 6533 | SLC6A6 | Y | 21366 | 0.673478 |
| 2661 | GDF9 | Y | 14566 | 0.664854 |
| 27252 | KLHL20 | | 226541 | 0.662994 |
| 204801 | NLRP11 | Y | | 0.655971 |
| 654790 | PCP4L1 | Y | 66425 | 0.655923 |

TABLE 9

| | | Known | | |
|---|---|---|---|---|
| | | Infertility | | Similarity |
| entrezGeneId | symbol | Association | MouseGeneId | (1-Distance) |
| 64377 | CHST8 | | 68947 | 1 |
| 2488 | FSHB | Y | 14308 | 0.807603 |
| 3972 | LHB | Y | 16866 | 0.799529 |
| 8022 | LHX3 | Y | 16871 | 0.720396 |
| 23373 | CRTC1 | Y | 382056 | 0.68314 |
| 2798 | GNRHR | Y | 14715 | 0.680513 |
| 7425 | VGF | Y | 381677 | 0.673726 |
| 54551 | MAGEL2 | | 27385 | 0.656467 |
| 1813 | DRD2 | Y | 13489 | 0.650742 |
| 5617 | PRL | Y | 19109 | 0.643812 |
| 1081 | CGA | Y | 12640 | 0.62561 |
| 5122 | PCSK1 | | 18548 | 0.624284 |
| 3763 | KCNJ6 | | 16522 | 0.624099 |
| 6447 | SCG5 | Y | 20394 | 0.611227 |
| 6833 | ABCC8 | Y | 20927 | 0.602154 |
| 9985 | REC8 | Y | 56739 | 0.592734 |
| 273 | AMPH | | 218038 | 0.592075 |
| 2688 | GH1 | | 14599 | 0.587602 |
| 4438 | MSH4 | Y | 55993 | 0.571955 |
| 113091 | PTH2 | | 114640 | 0.559548 |
| 11144 | DMC1 | Y | 13404 | 0.55841 |
| 25970 | SH2B1 | Y | 20399 | 0.55654 |
| 6658 | SOX3 | Y | 20675 | 0.553021 |
| 135935 | NOBOX | Y | 18291 | 0.551976 |
| 3990 | UPC | | 15450 | 0.550449 |

In an aspect of the invention, genetic loci are ranked according to their expression levels in humans and mice. For example, it is determined whether a biomarker is expressed in mice. If the biomarker is expressed in mice, the biomarker receives a higher ranking. If the biomarker is also expressed in humans, the biomarker is ranked even higher by the ranking system. If a biomarker is not expressed in mice, or in humans, it would receive a low ranking. A biomarker would receive the lowest ranking if it was expressed neither in mouse nor in human. Known methods in the art can be employed to rank genetic regions. It should be appreciated that any known ranking methodology can be utilized in the present invention, as discussed above. For example, the Friedman test, Kruskal-Wallis test, Spearman's rank correlation coefficient, Wilcoxon rank-sum test, and/or Wilcoxon signed-rank test are known statistical methods. The Friedman test is similar to the parametric repeated measures ANOVA; it is used to detect differences in treatments across multiple test attempts. The procedure involves ranking each row (or block) together, then considering the values of ranks by columns. See Friedman, Milton (December 1937). “The use of ranks to avoid the assumption of normality implicit in the analysis of variance”. Journal of the American Statistical Association (American Statistical Association) 32 (200): 675-701. Also, the Spearman's rank-order correlation is the nonparametric version of the Pearson product-moment correlation. Spearman's correlation coefficient measures the strength of association between two ranked variables. See Lehman, Ann (2005). Jmp For Basic Univariate And Multivariate Statistics: A Step-by-step Guide. Cary, N.C.: SAS Press. p. 123. The Wilcoxon signed-rank test is a non-parametric statistical hypothesis test used when comparing two related samples, matched samples, or repeated measurements on a single sample to assess whether their population mean ranks differ (i.e., it is a paired difference test). See Wilcoxon, Frank (December 1945). “Individual comparisons by ranking methods”. Biometrics Bulletin 1 (6): 80-83.

In an aspect of the invention, another possible ranking scheme employs listing genes in order from most to least statistically significant, when the correlation with phenotype in mice is determined. In this method, confidence intervals and p values are employed, where P-values<0.025 are considered statistically significant. A series of linear regression models are fit, where the outcome variable is the phenotype expression score for a given gene, and the independent variables are group (expressed phenotype v. control) and principal component derived ethnicity (for humans) or strain (for mice) (continuous). The p-value for group is used for statistical inference. The model is fit once for each gene.

In an aspect of the invention, another possible gene ranking scheme, genetic loci are ranked according to a Celmatix Fertilome™Score, G1Version2, that reflects the likelihood that a gene is involved in fertility or reproduction. This score is computed using a database of mined and curated data, containing attributes for each gene in the genome. These attributes include: diseases and disorders related to infertility, molecular pathways, molecular interactions, gene clusters, mouse phenotypes associated with each gene, gene expression data in reproductive tissues, proteomics data in oocytes, and accrued information from scientific publications through text-mining.

The process for ranking fertility-related attributes of a gene or genetic region (locus) to obtain a score is carried out by the SESMe algorithm. The SESMe algorithm is applied to a database of features and attributes that might make a particular gene important for fertility. The algorithm assigns a score and a relative weight to each feature to then rank genetic regions from most to least important (or vice versa) by weighting features and attributes associated with that genetic region. For example, a score is assigned to a gene by compiling the combined weighted values of attributes associated with that gene. After each gene is scored based on its weighted attributes, the genetic loci can be ranked in order of importance in accordance with their score. The weighted value for each infertility attribute may be scaled in any manner including and not limited to assigning a positive or negative integer to reflect the significance or severity of the attribute to infertility.

In certain embodiments, the weighted value for gene infertility attributes may be on a scale from −10 to +10. A +10 may indicate that an attribute of a gene being scored is highly associated with infertility because that attribute is prevalently found in infertile patient populations. A +4 may represent an attribute that is a latent infertility marker, meaning it will not cause infertility on its own, but may lead to infertility upon influence of external factors such as aging and smoking. Whereas +2 may represent an attribute found in some infertile patients but nothing directly relates the attribute to infertility. A zero on the scale may include an attribute not yet known to have any effect or any negative effect towards infertility. A −10 may include an attribute shown not to affect infertility whatsoever. Further, embodiments provide for the weighted scale to include a +1 for attributes that are commonly found in infertile patient populations, 0.5 for attributes similar to those found in infertile patient populations, and 0 for attributes without a causal link to infertility.

In addition, weighted values for attributes may be normalized based on the known significance of that attribute towards infertility. For example and in certain embodiments, when scoring attributes of a particular gene, each attribute may be assigned a 0 if the attribute is absent and a 1 if the attribute is present. The attributes may then be normalized based on the infertility significance of that attribute. For example, if the attribute is a genetic mutation known to be associated with infertility, then that attribute may be normalized by a factor of 5. In another example, if the attribute is a signaling pathway defect sometimes associated with infertility, then that attribute may be normalized by a factor of 2.

In an aspect of the invention, another possible gene ranking scheme involves the relative degree of infertility, subfertility, or premature decline in fertility risk associated with novel or common mutations or variants in a fertility gene. Genetic loci are ranked according to a Celmatix Fertilome™Score, G1Version3, that reflects the likelihood a gene is involved in fertility or reproduction. This score is computed using a database of mined and curated data, containing attributes for each gene in the genome. These attributes include: diseases and disorders related to infertility, molecular pathways, molecular interactions, gene clusters, mouse phenotypes associated with each gene, gene expression data in reproductive tissues, proteomics data in oocytes, and accrued information from scientific publications through text-mining. The Celmatix Fertilome™Score G1Version3 differs from G1Version2 because it contains more fertility genetic loci as an input for the score calculation.

### Mouse Model

The ability to engineer the mouse genome has proven useful for a variety of applications in research, medicine and biotechnology. Transgenic mice have become powerful reagents for modeling genetic disorders, understanding embryonic development and evaluating therapeutics. These mice and the cell lines derived from them have also accelerated basic research by allowing scientists to assign functions to genetic loci, dissect genetic pathways, and manipulate the cellular or biochemical properties of proteins.

Generation of a mouse model may be accomplished by any known method in the art. This can involve, but is not limited to, the addition of exogenous sequences of DNA to the genome of an animal during its earliest stage of development (the zygote) to permanently and heritably alter the expression of a particular gene or group of loci's expression. Methodologically, this can involve, but is not limited to, the pronuclear injection of short sequences of oligonucleotides derived in vitro, which replace endogeneous DNA sequences through homologous recombination and can therefore be designed to encode for mutated versions of genes or genetic regions. The generation of mouse models can also include, but is not limited to, the insertion of DNA sequences (designed to be expressed at an enhanced or attenuated level when compared to that of their endogenous copy) into retroviral vectors that allow the DNA sequences to replace their endogenous (normal) copy in the genome. See for example, Bedell, M. A., et al. Mouse models of human disease. Part I: Techniques and resources for genetic analysis in mice. Genes and Development 11, 1-10 (1997a); Rosenthal, N., & Brown, S. The mouse ascending: Perspectives for human-disease models. Nature Cell Biology 9, 993-999 (2007) doi:10.1038/ncb437; Yang, S. H. et al. Towards a transgenic model of Huntington's disease in a non-human primate. Nature 453, 921-924 (2008) doi:10.1038/nature06975; Yu, Y., & Bradley, A. Mouse genomic technologies: Engineering chromosomal rearrangements in mice. Nature Reviews Genetics 2, 780-790 (2001).

Using any or all of these methods, many different types of mutations can be introduced into any particular genetic region, including null or point mutations and complex chromosomal rearrangements such as large deletions, translocations, or inversions (Bedell et al., 1997a). Depending on the mutation introduced into the animal and as understood in the art, the geneticially modified animal may be referred to as a “knockin” or “knockout” animal, or the mutation itself may be referred to as a “knockin” mutation or “knockout” mutation.

Methods that target a particular genetic region for alteration in expression are particularly useful if a single gene is shown to be the primary cause of a disease., and indeed more than 3,000 genes have been targeted and altered in mice. Most of the targeted and altered genes have been related to disease (Hardouin & Nagy, 2000). Many genetically altered mice have similar, if not identical, phenotypes to human patients with lesions in the same/related genetic regions. Many mouse models therefore represent useful tools with which to model human disease.

In an aspect of the invention therefore, genetic loci that are identified as being highly ranked in association with particular aspects of infertility or reproductive biology and have previously never been directly associated with those characteristics in humans or in mice, would serve as good candidates for the generation of mouse models for infertility. These mouse models would in turn provide tools for testing therapeutic agents designed to overcome certain aspects of infertility related to particular molecular aetiologies.

### Testing of Therapeutic Agents

The genetically altered mouse is then assessed to determine whether the gene or biomarker expresses a phenotype. Genetically-altered test animals that show presence of an infertility phenotype are useful for therapeutic testing. For example, a genetically altered mouse expressing a phenotype can be dosed or exposed to a therapeutic agent such as, Human Chorionic Gonadotropin (hCG), (such as Pregnyl, Novarel, Ovidrel, and Profasi); Follicle Stimulating Hormone (FSH), (such as Follistim, Fertinex, Bravelle, and Gonal-F); Human Menopausal Gonadotropin (hMG), (such as Pergonal, Repronex, and Metrodin) or Gonadotropin Releasing Hormone (GnRH), (such as Factrel and Lutrepulse); Gonadotropin Releasing Hormone Agonist (GnRH agonist), (such as Lupron, Zoladex, and Synarel); or Gonadotropin Releasing Hormone Antagonist (GnRH antagonist), (such as Antagon and Cetrotide) to determine if the therapeutic agent is effective at overcoming infertility. A therapeutic agent that rescues the phenotype, i.e., returns or partially re-establishes the wild type fertility phenotype, is a good drug candidate.

### Predictive Value

Infertility may not be the result of a single genomic alteration, but rather may be the result of a combination of multiple factors or multiple alterations. Methods of the invention provide a better understanding of the molecular pathways underlying human fertility. For example, presence of an infertility-associated phenotype is used as a factor in ranking the importance of a gene in a database of genes associated with infertility in humans by associated the gene (or more often a mutation) with the phenotype. A correlation between the presence of an allele or a mutation in a gene with phenotype increases or decreases the predictive value of the contribution of the genomic region to phenotype.

### Computer Systems

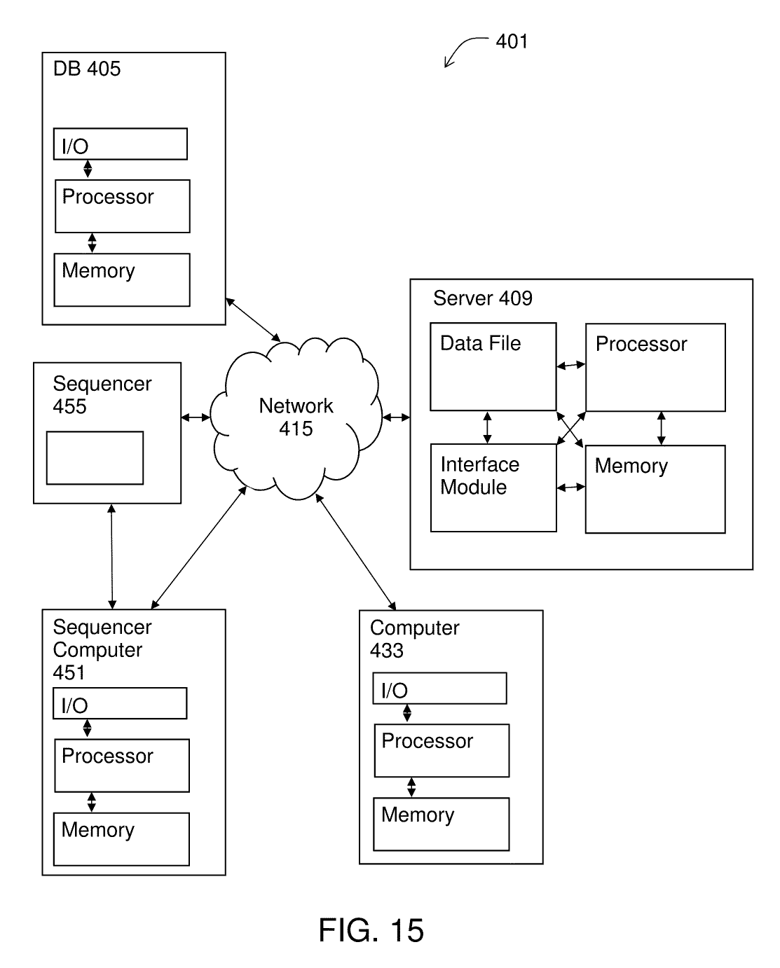

FIG. 15 illustrates a computer system 401 useful for implementing methodologies described herein. A system of the invention may include any one or any number of the components shown in FIG. 15. Generally, a system 401 may include a computer 433 and a server computer 409 capable of communication with one another over network 415. Additionally, data may optionally be obtained from a database 405 (e.g., local or remote). In some embodiments, systems include an instrument 455 for obtaining sequencing data, which may be coupled to a sequencer computer 451 for initial processing of sequence reads.

In some embodiments, methods are performed by parallel processing and server 409 includes a plurality of processors with a parallel architecture, i.e., a distributed network of processors and storage capable of collecting, filtering, processing, analyzing, ranking genetic data obtained through methods of the invention. The system may include a plurality of processors configured to, for example, 1) collect genetic data from different modalities: a) one or more infertility databases 405 (e.g. infertility databases, including private and public fertility-related data), b) from one or more sequencers 455 or sequencing computers 451, c) from mouse modeling, etc; 2) filter the genetic data to identify genetic variations; 3) associate genetic variations with infertility using methods described throughout the application (e.g., filtering, clustering, etc.); 4) determine statistical significance of genetic variations based on fertility criteria defined herein (e.g., Example 18); and 5) characterize/identify the genetic variations as infertility biomarkers.

By leveraging genetic data sets obtained across different sources, applying layers of analyses (i.e., filtering, clustering, etc.) to genetic data, and quantifying/qualifying statistical significance of that genetic data, systems of the invention are able yield and identify new infertility biomarkers that previously could not be determined to have any association with infertility. For example, methods of the invention utilize data sets from different modalities. The data sets range include data obtained from infertility databases (e.g., public and private), sequencing data (e.g., whole genome sequencing from one or more biological samples), and genetic data obtained from mouse modeling, etc. Several layers of analysis are then applied to the genetic data to identify whether variations are potentially associated with infertility. Particularly, the genetic data sets are subject to evolutionary conservation analysis, filtering analysis (see FIG. 5) and/or subject to clustering analysis. After those analyses are applied, the variants potentially associated with infertilty are then assessed for biological and statistical significance. The variants that are determined to be statistically significant are then classified as infertility biomarkers, even if those variant had no prior association with infertility. Accordingly, using the invention's multi-modal and layered analysis, one is able to identify infertility biomarkers that would not have been identified or associated with infertility using standard techniques (i.e. comparing genetic sequences of an abnormal, infertile population to genetic sequences of a normal, fertile population).

While other hybrid configurations are possible, the main memory in a parallel computer is typically either shared between all processing elements in a single address space, or distributed, i.e., each processing element has its own local address space. (Distributed memory refers to the fact that the memory is logically distributed, but often implies that it is physically distributed as well.) Distributed shared memory and memory virtualization combine the two approaches, where the processing element has its own local memory and access to the memory on non-local processors. Accesses to local memory are typically faster than accesses to non-local memory.

Computer architectures in which each element of main memory can be accessed with equal latency and bandwidth are known as Uniform Memory Access (UMA) systems. Typically, that can be achieved only by a shared memory system, in which the memory is not physically distributed. A system that does not have this property is known as a Non-Uniform Memory Access (NUMA) architecture. Distributed memory systems have non-uniform memory access.

Processor-processor and processor-memory communication can be implemented in hardware in several ways, including via shared (either multiported or multiplexed) memory, a crossbar switch, a shared bus or an interconnect network of a myriad of topologies including star, ring, tree, hypercube, fat hypercube (a hypercube with more than one processor at a node), or n-dimensional mesh.

Parallel computers based on interconnected networks must incorporate routing to enable the passing of messages between nodes that are not directly connected. The medium used for communication between the processors is likely to be hierarchical in large multiprocessor machines. Such resources are commercially available for purchase for dedicated use, or these resources can be accessed via “the cloud,” e.g., Amazon Cloud Computing.

A computer generally includes a processor coupled to a memory and an input-output (I/O) mechanism via a bus. Memory can include RAM or ROM and preferably includes at least one tangible, non-transitory medium storing instructions executable to cause the system to perform functions described herein. As one skilled in the art would recognize as necessary or best-suited for performance of the methods of the invention, systems of the invention include one or more processors (e.g., a central processing unit (CPU), a graphics processing unit (GPU), etc.), computer-readable storage devices (e.g., main memory, static memory, etc.), or combinations thereof which communicate with each other via a bus.

A processor may be any suitable processor known in the art, such as the processor sold under the trademark XEON E7 by Intel (Santa Clara, Calif.) or the processor sold under the trademark OPTERON 6200 by AMD (Sunnyvale, Calif.).

Input/output devices according to the invention may include a video display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

### EXAMPLES

### Example 1

### Identification of Oocyte Proteins

Oocytes are collected from females, for example mice, by superovulation, and zona pellucidae are removed by treatment with acid Tyrode solution. Oocyte plasma membrane (oolemma) proteins exposed on the surface can be distinguished at this point by biotin labeling. The treated oocytes are washed in 0.01 M PBS and treated with lysis buffer (7 M urea, 2 M thiourea, 4% (w/v) 3-[(3-cholamidopropyl)dimethylammonio]-1-propanesulfonate (CHAPS), 65 mM dithiothreitol (DTT), and 1% (v/v) protease inhibitor at −80° C.). Oocyte proteins are resolved by one-dimensional or two-dimensional SDS-PAGE. The gels are stained, visualized, and sliced. Proteins in the gel pieces are digested (12.5 ng/μl trypsin in 50 mM ammonium bicarbonate overnight at 37° C.), and the peptides are extracted and microsequenced.

### Example 2

### Sample Population for Identification of Infertility-Related Polymorphisms

Genomic DNA is collected from 30 female subjects (15 who have failed multiple rounds of IVF versus 15 who were successful). In particular, all of the subjects are under age 38. Members of the control group succeeded in conceiving through IVF. Members of the test group have a clinical diagnosis of idiopathic infertility, and have failed three of more rounds of IVF with no prior pregnancy. The women are able to produce eggs for IVF and have a reproductively normal male partner. To focus on infertility resulting from oocyte defects (and eliminate factors such as implantation defects) women who have subsequently conceived by egg donation are favored.

### Example 3

### Sample Population for Identification of Infertility-Related Polymorphisms

In a follow-up study of a larger cohort, genomic DNA is collected from 300 female subjects (divided into groups having profiles similar to the groups described above). The DNA sequence polymorphisms to be investigated are selected based on the results of small initial studies.

### Example 4

### Sample Population for Identification of Premature Ovarian Failure (POF) and Premature Maternal Aging Polymorphisms

Genomic DNA is collected from 30 female subjects who are experiencing symptoms of premature decline in egg quality and reserve including abnormal menstrual cycles or amenorrhea. In particular, all of the subjects are between the ages of 15-40 and have follicle stimulating hormone (FSH) levels of over 20 international units (IU) and a basal antral follicle count of under 5. Members of the control group succeeded in conceiving through IVF. Members of the test group have no previous history of toxic exposure to known fertility damaging treatments such as chemotherapy. Members of this group may also have one or more female family member who experienced menopause before the age of 40.

### Example 5

### Sample Procurement and Preparation

Blood is drawn from patients at fertility clinics for standard procedures such as gauging hormone levels and many clinics bank this material after consent for future research projects. Although DNA is easily obtained from blood, wider population sampling is accomplished using home-based, noninvasive methods of DNA collection such as saliva using an Oragene DNA self collection kit (DNA Genotek).

Blood samples—Three-milliliter whole blood samples are venously collected and treated with sodium citrate anticoagulant and stored at 4° C. until DNA extraction.

Whole Saliva—Whole saliva is collected using the Oragene DNA selfcollection kit following the manufacturer's instructions. Participants are asked to rub their tongues around the inside of their mouths for about 15 sec and then deposit approximately 2 ml saliva into the collection cup. The collection cup is designed so that the solution from the vial.'s lower compartment is released and mixes with the saliva when the cap is securely fastened. This starts the initial phase of DNA isolation, and stabilizes the saliva sample for long-term storage at room temperature or in low temperature freezers. Whole saliva samples are stored and shipped, if necessary, at room temperature. Whole saliva has the potential advantage over other non-invasive DNA sampling methods, such as buccal and oral rinse, of providing large numbers of nucleated cells (eg., epithelial cells, leukocytes) per sample.

Blood clots—Clotted blood that is usually discarded after extraction through serum separation, for other laboratory tests such as for monitoring reproductive hormone levels is collected and stored at −80° C. until extraction.

Sample Preparation—Genomic DNA is prepared from patient blood or saliva for downstream sequencing applications with commercially available kits (e.g., Invitrogen's ChargeSwitch® gDNA Blood Kit or DNA Genotek kits, respectively). Genomic DNA from clotted is prepared by standard methods involving proteinase K digestion, salt/chloroform extraction and 90% ethanol precipitation of DNA. (see N Kanai et al., 1994, “Rapid and simple method for preparation of genomic DNA from easily obtainable clotted blood,” J Clin Pathol 47:1043-1044, which is incorporated by reference in its entirety for all purposes).

### Example 6

### Manufacturing of a Customized Oligonucleotide Library

A customized oligonucleotide library can be used to enrich samples for DNAs of interest. Several methods for manufacturing customized oligonucleotide libraries are known in the art. In one example, Nimblegen sequence capture custom array design is used to create a customized target enrichment system tailored to infertility related genetic loci. A customized library of oligonucleotides is designed to target genetic regions of Tables 1-7. The custom DNA oligonucleotides are synthesized on a high density DNA Nimblegen Sequence Capture Array with Maskless Array Synthesizer (MAS) technology. The Nimblegen Sequence Capture Array system workflow is array based and is performed on glass slides with an X1 mixer (Roche NimbleGen) and the NimbleGen Hybridization System.

In a similar example, Agilent's eArray (a web-based design tool) is used to create a customized target enrichment system tailored to infertility related genetic loci. The SureSelect Target Enrichment System workflow is solution-based and is performed in microcentrifuge tubes or microtiter plates. A customized oligonucleotide library is used to enrich samples for DNA of interest. Agilent's eArray (a web-based design tool) is used to create a customized target enrichment system tailored to infertility related genetic loci. A customized library is designed to target genetic regions of Tables 1-7. The custom RNA oligonucleotides, or baits, are biotinylated for easy capture onto streptavidin-labeled magnetic beads and used in Agilent's SureSelectTarget Enrichment System. The SureSelect Target Enrichment System workflow is solution-based and is performed in microcentrifuge tubes or microtiter plates.

### Example 7

### Capture of Genomic DNA

Genomic DNA is sheared and assembled into a library format specific to the sequencing instrument utilized downstream. Size selection is performed on the sheared DNA and confirmed by electrophoresis or other size detection method.

Several methods to capture genomic DNA are known in the art. In one example, the size-selected DNA is purified and the ends are ligated to annealed oligonucleotide linkers from Illumina to prepare a DNA library. DNA-adaptor ligated fragments are hybridized to a Nimblegen Sequence Capture array using an X1 mixer (Roche NimbleGen) and the Roche NimbleGen Hybridization System. After hybridization, are washed and DNA fragments bound to the array are eluted with elution buffer. The captured DNA is then dried by centrifugation, rehydrated and PCR amplified with polymerase. Enrichment of DNA can be assessed by quantitative PCR comparison to the same sample prior to hybridization.

In a similar example, the size-selected DNA is incubated with biotinylated RNA oligonucleotides “baits” for 24 hours. The RNA/DNA hybrids are immobilized to streptavidin-labeled magnetic beads, which are captured magnetically. The RNA baits are then digested, leaving only the target selected DNA of interest, which is then amplified and sequenced.

### Example 8

### Sequencing of Target Selected DNA

Target-selected DNA is sequenced by a paired end (50 bp) re-sequencing procedure using Illumina.'s Genome Analyzer. The combined DNS targeting and resequencing provides 45 fold redundancy which is greater than the accepted industry standard for SNP discovery.

### Example 9

### Correlation of Polymorphisms with Fertility

Polymorphisms among the sequences of target selected DNA from the pool of test subjects are identified, and may be classified according to where they occur in promoters, splice sites, or coding regions of a gene. Polymorphisms can also occur in regions that have no apparent function, such as introns and upstream or downstream non-coding regions. Although such polymorphisms may not be informative as to the functional defect of an allele, nevertheless, they are linked to the defect and useful for predicting infertility. The polymorphisms are analyzed statistically to determine their correlation with the fertility status of the test subjects. The statistical analysis indicates that certain polymorphisms identify gene defects that by themselves (homozygous or heterozygous) are sufficient to cause infertility. Other polymorphisms identify genetic variants that reduce, but do not eliminate fertility. Other polymorphisms identify genetic variants that have an apparent effect on fertility only in the presence of particular variants of other genetic loci. Other polymorphisms identify genetic variants that have an apparent effect on fertility only in the presence of particular phenotypes. Other polymorphisms identify genetic variants that have an apparent effect on fertility only in the presence of particular environmental exposures. Still other polymorphisms identify genetic variants that have an apparent effect on fertility only in the presence of any combination of particular variants of other genetic loci, presence of particular phenotypes, and particular environmental exposures.

### Example 10

### Correlation of Polymorphisms with Premature Ovarian Failure (POF)

Polymorphisms among the sequences of target selected DNA from the pool of test subjects are identified, and may be classified according to where they occur in promoters, splice sites, or coding regions of a gene. Polymorphisms can also occur in regions that have no apparent function, such as introns and upstream or downstream non-coding regions. Although such polymorphisms may not be informative as to the functional defect of an allele, nevertheless, they are linked to the defect and useful for predicting likelihood of premature ovarian failure (POF). The polymorphisms are analyzed statistically to determine their correlation with the POF status of the test subjects. The statistical analysis indicates that certain polymorphisms identify gene defects that by themselves (homozygous or heterozygous) are sufficient to cause POF. Other polymorphisms identify genetic variants that increase the likelihood, but do not cause POF. Other polymorphisms identify genetic variants that have an apparent effect on POF only in the presence of particular variants of other genetic loci. Other polymorphisms identify genetic variants that have an apparent effect on POF only in the presence of particular phenotypes. Other polymorphisms identify genetic variants that have an apparent effect on POF only in the presence of particular environmental exposures. Still other polymorphisms identify genetic variants that have an apparent effect on POF only in the presence of any combination of particular variants of other genetic loci, presence of particular phenotypes, and particular environmental exposures.

### Example 11

### Correlation of Polymorphisms with Premature Maternal Aging

Polymorphisms among the sequences of target selected DNA from the pool of test subjects are identified, and may be classified according to where they occur in promoters, splice sites, or coding regions of a gene. Polymorphisms can also occur in regions that have no apparent function, such as introns and upstream or downstream non-coding regions. Although such polymorphisms may not be informative as to the functional defect of an allele, nevertheless, they are linked to the defect and useful for predicting likelihood of premature decline in ovarian reserve and egg quality (i.e., maternal aging). The polymorphisms are analyzed statistically to determine their correlation with the maternal aging status of the test subjects. The statistical analysis indicates that certain polymorphisms identify gene defects that by themselves (homozygous or heterozygous) are sufficient to cause premature maternal aging. Other polymorphisms identify genetic variants that increase the likelihood, but do not cause premature maternal aging. Other polymorphisms identify genetic variants that have an apparent effect on premature maternal aging only in the presence of particular variants of other genetic loci. Other polymorphisms identify genetic variants that have an apparent effect on premature maternal aging only in the presence of particular phenotypes. Other polymorphisms identify genetic variants that have an apparent effect on premature maternal aging only in the presence of particular environmental exposures. Still other polymorphisms identify genetic variants that have an apparent effect on premature maternal aging only in the presence of any combination of particular variants of other genetic loci, presence of particular phenotypes, and particular environmental exposures.

### Example 12

### Diagnostics and Counseling

A library of nucleic acids in an array format is provided for infertility diagnosis. The library consists of selected nucleic acids for enrichment of genetic targets wherein polymorphisms in the targets are correlated with variations in fertility. A patient nucleic acid sample (appropriately cleaved and size selected) is applied to the array, and patient nucleic acids that are not immobilized are washed away. The immobilized nucleic acids of interest are then eluted and sequenced to detect polymorphisms. According to the polymorphisms detected, the fertility status of the patient is evaluated and/or quantified. The patient is accordingly advised as to the suitability and likelihood of success of a fertility treatment or suitability or necessity of a particular in vitro fertilization procedure.

### Example 13

### Diagnostics and Counseling

A complete DNA sequence of any number of or all of the genes in Tables 1-7 is determined using a targeted resequencing protocol. According to the polymorphisms detected and the phenotypic traits and environmental exposures reported, the fertility status of the patient is evaluated and/or quantified. The patient is accordingly advised as to the suitability and likelihood of success of a fertility treatment or suitability or necessity of a particular in vitro fertilization procedure.

### Example 14

### Diagnostics and Counseling

A library of nucleic acids in an array format is provided for infertility diagnosis. The library consists of selected nucleic acids for enrichment of genetic targets wherein polymorphisms in the targets are correlated with variations in fertility. A patient nucleic acid sample (appropriately cleaved and size selected) is applied to the array, and patient nucleic acids that are not immobilized are washed away. The immobilized nucleic acids of interest are then eluted and sequenced to detect polymorphisms. According to the polymorphisms detected and the phenotypic traits and environmental exposures reported, the POF status of the patient or likelihood of future POF occurrence is evaluated and/or quantified. The patient is accordingly advised as to whether preventative egg or ovary preservation is indicated.

### Example 15

### Diagnostics and Counseling

A complete DNA sequence of any number of or all of the genes in Tables 1-7 is determined using a targeted resequencing protocol. According to the polymorphisms detected and the phenotype and environmental exposures reported, the fertility status of the patient is evaluated and/or quantified. According to the polymorphisms detected and the phenotypic traits and environmental exposures reported, the POF status of the patient or likelihood of future POF occurrence is evaluated and/or quantified. The patient is accordingly advised as to whether preventative egg or ovary preservation is indicated.

### Example 16

### Diagnostics and Counseling

A library of nucleic acids in an array format is provided for infertility diagnosis. The library consists of selected nucleic acids for enrichment of genetic targets wherein polymorphisms in the targets are correlated with variations in fertility. A patient nucleic acid sample (appropriately cleaved and size selected) is applied to the array, and patient nucleic acids that are not immobilized are washed away. The immobilized nucleic acids of interest are then eluted and sequenced to detect polymorphisms. According to the polymorphisms detected and the phenotypic traits and environmental exposures reported, the maternal aging status of the patient or likelihood of future premature maternal aging occurrence is evaluated and/or quantified. The patient is accordingly advised as to whether preventative egg or ovary preservation, minimization of certain environmental exposures such as alcohol intake or smoking, or mitigation of certain phenotypes such as having children at a younger age is indicated.

### Example 17

### Diagnostics and Counseling

A complete DNA sequence of any number of or all of the genes in Tables 1-7 is determined using a targeted resequencing protocol. According to the polymorphisms detected and the phenotypic traits and environmental exposures reported, the fertility status of the patient is evaluated and/or quantified. According to the polymorphisms detected and the phenotype and environmental exposures reported, the maternal aging status of the patient or likelihood of future premature maternal aging occurrence is evaluated and/or quantified. The patient is accordingly advised as to whether preventative egg or ovary preservation, minimization of certain environmental exposures such as alcohol intake or smoking, or mitigation of certain phenotypes such as having children at a younger age is indicated.

### Example 18

### Whole Genome Sequencing for Female Infertility Biomarker Discovery

Whole genome sequencing (WGS) allows one to characterize the complete nucleic acid sequence of an individual's genome. With the amount of data obtained from WGS, a comprehensive collection of an individual's genetic variation is obtainable, which provides great potential for genetic biomarker discovery. The data obtained from WGS can be advantageously used to expand the ability to identify and characterize female infertility biomarkers. However, the ability to identify unknown variations of fertility significance within the vast WGS datasets is a challenging task that is analogous to finding a needle in a haystack.

Methods of the invention, according to certain embodiments, rely on bioinformatics to filter through WGS data in order to identify and prioritize variations of infertility significance. Specifically, the invention relies on a combination of clinical phenotypic data and an infertility knowledgebase to rank and/or score genomic regions of interest and their likely impact on different fertility disorders. In certain aspects, the filtering approach involves assessing sequencing data to identify genomic variations, identifying at least one of the variations as being in a genomic region associated with infertility, determining whether the at least one variation is a biologically-significant variation and/or a statistically-significant variation, and characterizing at least one identified variation as an infertility biomarker based on the determining step. A genomic region associated with infertility is any DNA sequence in which variation is associated with a change in fertility. Such regions may include genes (e.g., any region of DNA encoding a functional product), genetic regions (e.g., regions including genes and intergenic regions with a particular focus on regions conserved throughout evolution in placental mammals), and gene products (e.g., RNA and protein). In particular embodiments, the infertility-associated genetic region is a maternal effect gene, as described above. In particular embodiments, the infertility-associated genetic region is a gene (including exons, introns, and evolutionarily conserved regions of DNA flanking either side of said gene) that impacts fertility.

This filtering approach facilitates rapid identification of functionally relevant variants within genomic regions of significance for fertility. The identified variations with infertility significance obtained from WGS data may be used in diagnostic testing, and ultimately assist physicians in data interpretation, guide fertility therapeutics, and clarify why some patients are not responding to treatment. The following illustrates use of WGS data to identify variants of interest in accordance with methods of the invention.

FIG. 5 generally illustrates filtering through variations obtained from WGS sequencing data in order to identify variations of infertility significance. As shown in FIG. 5, the first step is to identify sequence variants in whole genome sequence. A typical whole genome can include up to four million variants. The next filtering step involves eliminating variants outside of regions of interest for female fertility (which amounts to about one million variants). Next, the filtering method isolates variants within regions of interest for female fertility, which is described herein as Fertilome nucleic acid (i.e., regions of the human genome that control egg quality and fertility). Variations located within the Fertilome nucleic acid may be in the 100,000s. The variations within the Fertilome nucleic acid are further filtered to identify and score variations of infertility significance (such variations are typically present in double digits). Particularly, variations of infertility significance include those within regions predicted to effect biological function or that show a statistical correlation to infertility or treatment failure.

Biologically-significant variations within the Fertilome nucleic acid include mutations that result in a change: 1) to a different amino acid predicted to alter the folding and/or structure of the encoded protein, 2) to a different amino acid occurring at a site with high evolutionarily conservation in mammals, 3) that introduces a premature stop termination signal, 4) that causes a stop termination signal to be lost, 5) that introduces a new start codon, 6) that causes a start codon to be lost or 7) that disrupts a splicing signal. Statistically-significant variations within the Fertilome nucleic acid are described in relation to and listed in Tables 2 and 3. Other methods for classifying variations as statistically- or biologically-significant includes scoring variations using an infertility knowledgebase (which is described in relation to Tables 5-7 above and FIG. 6 below). The infertility knowledgebase ranks genetic loci based on attributes associated with infertility. The attributes include: diseases and disorders related to infertility, molecular pathways, molecular interactions, gene clusters, mouse phenotypes associated with each gene, gene expression data in reproductive tissues, proteomics data in oocytes, and accrued information from scientific publications through text-mining. List of ranked genes of interest are provided in Tables 5-7.

FIG. 6 illustrates various data sources integrated into the infertility knowledgebase for analyzing whole-genome sequencing data according to certain embodiments. As shown in FIG. 6, information is obtained from private and public fertility-related data. Private and/or public fertility-related data may include genetic loci that regulate processes of implantation, idiopathic infertility genetic loci, polycystic ovary syndrome (PCOS) genetic loci, egg quality genetic loci, endometriosis genetic loci, and premature ovarian failure genetic loci. The private and/or public fertility-related data is then subjected to the ABCoRE Algorithm to provide genomic regions and variations of interest that can be introduced into a fertility database evidence matrix along with other fertility-related information. As described in the detailed description, the ABCoRE algorithm identifies fertility regions of interest by performing evolutionary conservation analysis of one or more genetic loci obtained from the private and/or public fertility-related data. The other fertility-related information includes, for example, protein-protein interactions, pathway interactions, gene orthologs and paralogs, genomic “hotpsots”, gene protein expression and meta-analysis, and data from genomic studies. In operation, whole genomic sequencing data is compared to the compiled data in the fertility database evidence matrix to facilitate identification of potential genetic regions important for fertility. The fertility database evidence matrix filters through WGS variants to identify variants of fertility significance. In certain embodiments, the whole genomic sequencing data is also subjected to the SESMe algorithm that ranks each genetic region from most to least important for different aspects of female fertility.

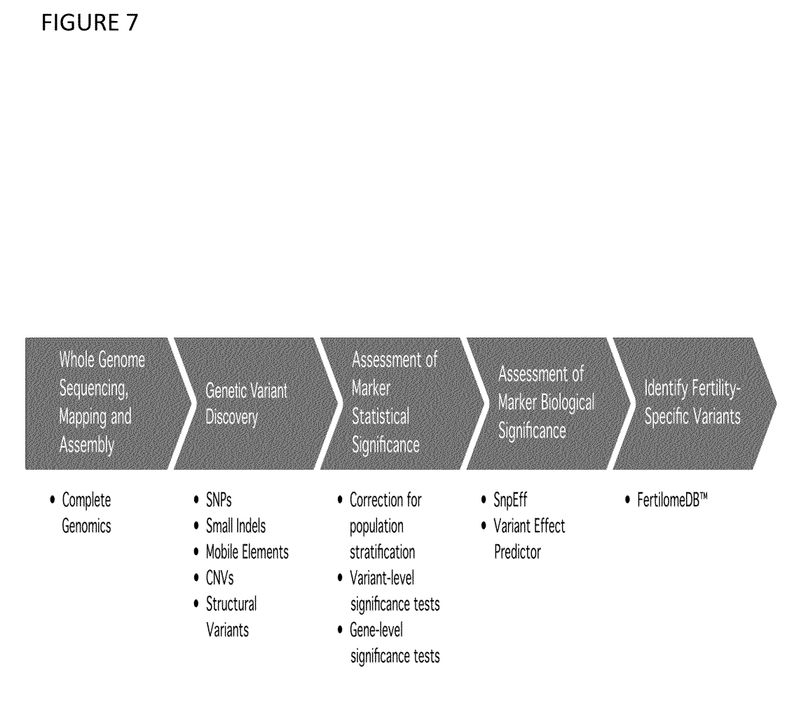

FIG. 7 illustrates a bioinformatics pipeline used to filter through WGS data to identify biomarkers associated with infertility according to certain embodiments. As shown in FIG. 7, samples are subjected to whole genome sequencing, mapping, and assembly. The WGS data is then analyzed to discover genetic variants such as SNPs, small indels, mobile elements, copy number variations, and structural variations. The identified variations are then assessed for statistical significance (See, for example, Tables 2 and 3 above). This includes correction for population stratification, variation-level significance tests, and gene level significance tests. In addition, the biological significance of WGS variants is determined using the SnpEff and Variant Effect Predictor (www.ensembl.org) engines (See, for example, Table 1 above). Variants of biological and statistical significance are then entered into the infertility knowledgebase (i.e., Fertilome database) in order to classify those variants as fertility biomarkers.

The following illustrates use of WGS data to identify variants of interest in accordance with methods of the invention.

Samples were collected from female patients undergoing fertility treatment at an academic reproductive medical center, and categorized into idiopathic infertility or primary ovarian insufficiency (POI) study groups. Phenotypic information was collected for each patient by mining >200 variables from electronic health records. Genomic DNA extracted from blood samples underwent WGS by Complete Genomics (Mountain View, Calif.). Analysis of genetic variants from WGS was assisted by an infertility knowledgebase with >800 genomic regions of interest (ROI) ranked by a scoring algorithm predicting their likely impact on different fertility disorders, based on publications, data repositories (including protein-protein interactions and tissue expression patterns), meta-analyses of these data, and animal model phenotypes.

The collected female samples were subjected to the processes/algorithms depicted in FIGS. 5-7 (described in more detail above). With those female samples, approximately 50,000 novel variants (approximately 1.6% of total variants observed) were identified as having fertility significances that have not been previously reported in databases such as the sbSNP reference. The identified fertility-related variants included single nucleotide polymorphisms (SNPs, insertions, deletions, copy number variations, inversions, and translocations. Of the SNPs, some of them are predictive to have putative functional significance based on the knowledgebase. For example, the knowledgebase scored some SNPs as deleterious mutations due to potential loss of function or changes in protein structure.

In certain aspects, the genomic data, such as WGS data, of a patient/subject population is subjected to a population stratification correction. Population stratification correction accounts for the presence of a systematic difference in allele frequencies between subpopulations in a population possibly due to different ancestry. When conducting population stratification, data is compared to a number (e.g., 1,000) of ethnically diverse individuals as part of the 1000 Genomes Project (100G). Principal components analysis (PCA) is applied to model and identify ancestry differences. In addition, computed association statistics are adjusted for the first two principal components.

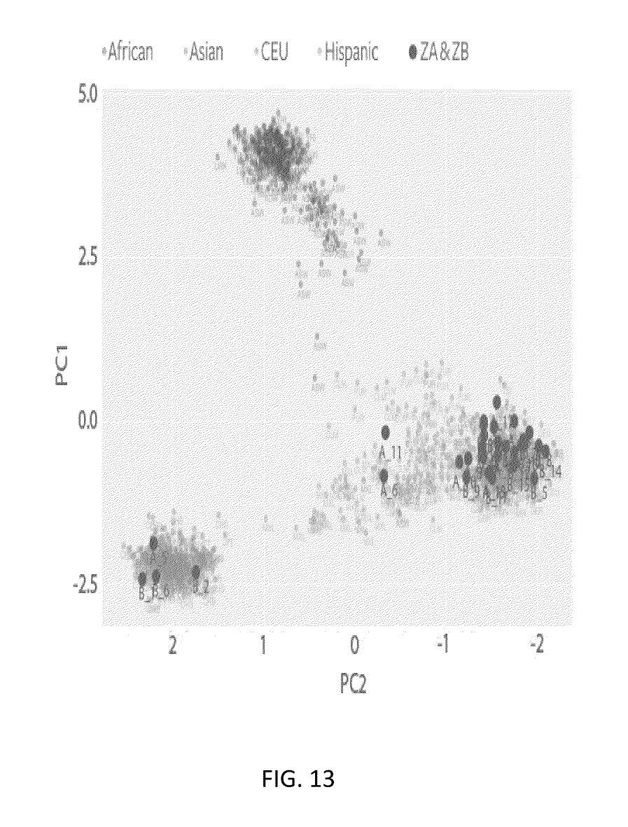

FIG. 13 illustrates population stratification correction of two patient groups. The patient groups include female patients undergoing non-donor in vitro fertilization (IVF) cycles. The patients were 38 years old or younger at the time of enrollment, and had no history of carrying a pregnancy beyond the first term before IVF treatment. Each patient had lack of an apparent cause for infertility (i.e., unexplained) after an evaluation of a complete medical history, physical examination, endocrine profile, and the results of an intimate partner's sperm analysis. The patients were divided into two groups. Group A included 11 patients that experienced no live birth or pregnancy beyond the first trimester after 3 or more IVF cycles. Group B included 18 patients that experienced live birth or pregnancy beyond the first trimester through use of IVF therapy. With population stratification correction, Group A and B patients cluster (are shown as black dots) with East Asian, African, Hispanic, and European individuals as shown in the principal component analysis chart of FIG. 13. This data shows that ethnicity may be linked to infertility, or that certain genomic variations are more prevalent in certain ethnic populations. Accordingly, aspects of the invention involve assessing ethnicity of an individual, either through self-reporting by the individual (e.g., by a questionnaire) or via an assay that looks for known biomarkers related to genetic ethnicity of an individual. That ethnicity data (genetic or self-reported) may be used to guide testing, such as by ensuring that certain genomic variations are checked that are known to be associated with certain ethnic populations.

### Example 19

Approximately 15% of couples experiencing difficulty conceiving are diagnosed with idiopathic infertility. Genetic polymorphisms could shed light on many of these currently unexplained cases by revealing disruptions to oocyte quality or uterine receptivity that may exist on a subcellular level.

In accordance with certain aspects, copy number variations are examined for their effect on female fertility using comparative genomic hybridization (CGH) arrays. CGH provides for methods of determining the relative number of copies of nucleic acid sequences in one or more subject genomes or portions thereof (for example, an infertility marker) as a function of the location of those sequences in a reference genome (for example, a normal human genome). As a result, CGH provides a map of losses and gains in nucleic acid copy number across the entire genome without prior knowledge of specific chromosomal abnormalities. Methods of the invention capitalize on the ability to detect copy number variations without the need for prior knowledge in order to detect potential mutations with infertility significance within patient populations that have unexplained infertility.

The following illustrates use of CGH arrays to identify copy number variants of interest in accordance with methods of the invention.

The study examined female patients undergoing non-donor in vitro fertilization (IVF) cycles. The patients were 38 years old or younger at the time of enrollment, and had no history of carrying a pregnancy beyond the first term before IVF treatment. Each patient had lack of an apparent cause for infertility (i.e., unexplained) after an evaluation of a complete medical history, physical examination, endocrine profile, and the results of an intimate partner's sperm analysis. The patients were divided into two groups. Group A included 11 patients that experienced no live birth or pregnancy beyond the first trimester after 3 or more IVF cycles. Group B included 18 patients that experienced live birth or pregnancy beyond the first trimester through use of IVF therapy.

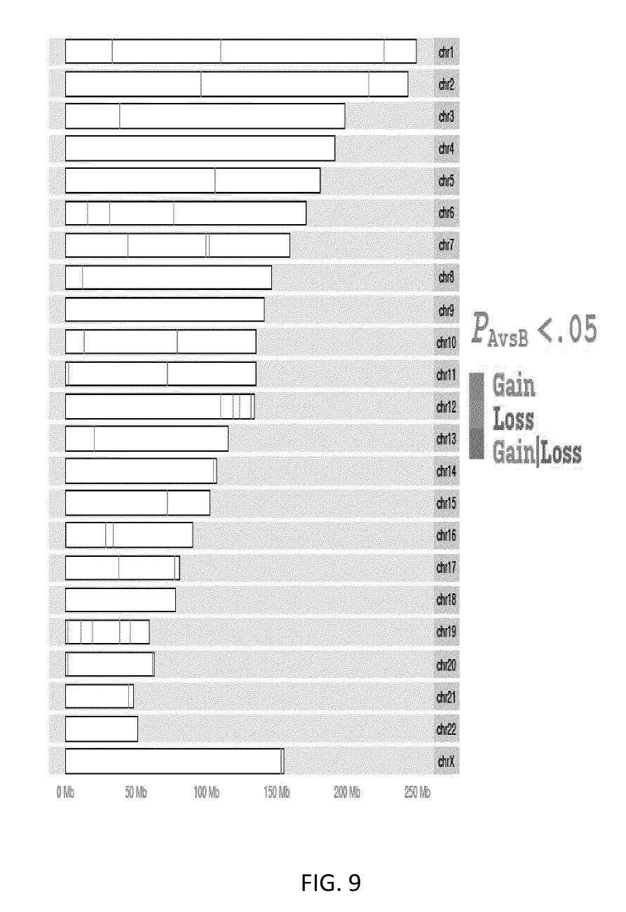

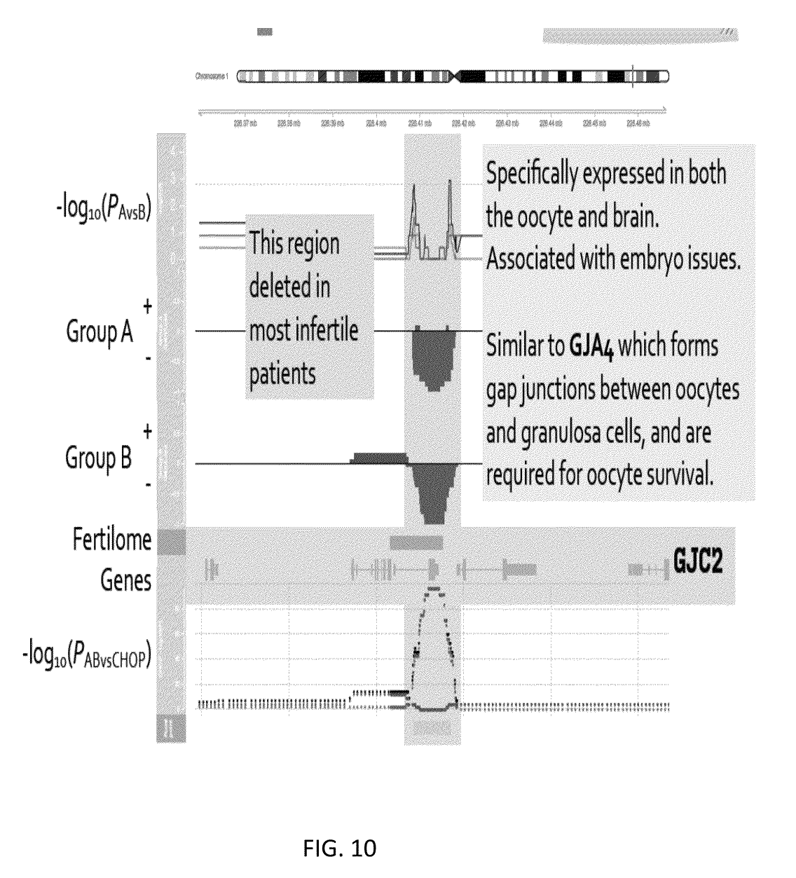

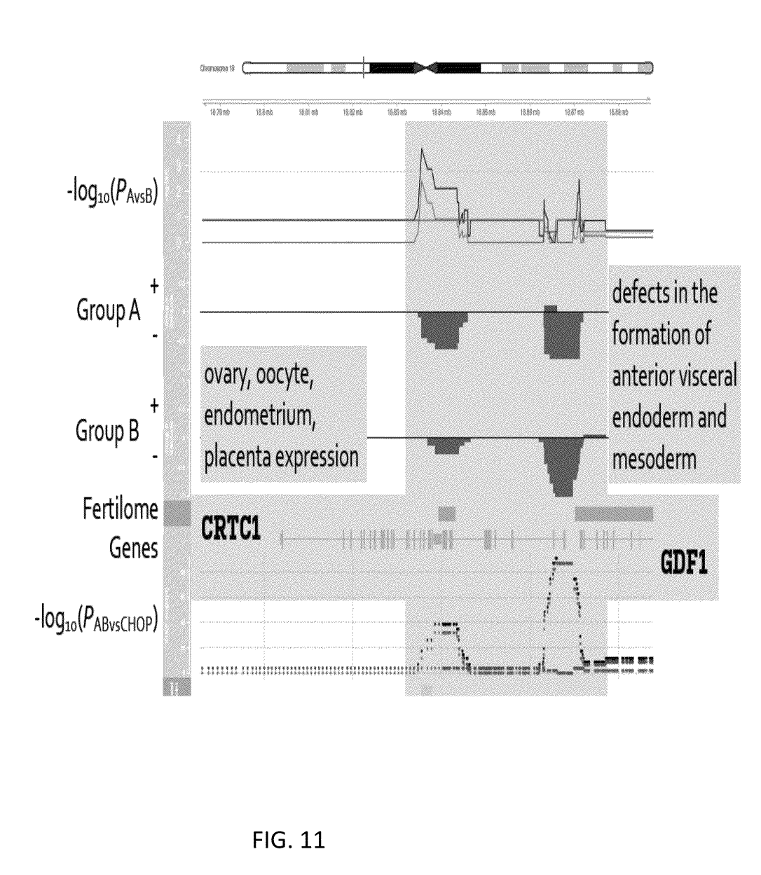

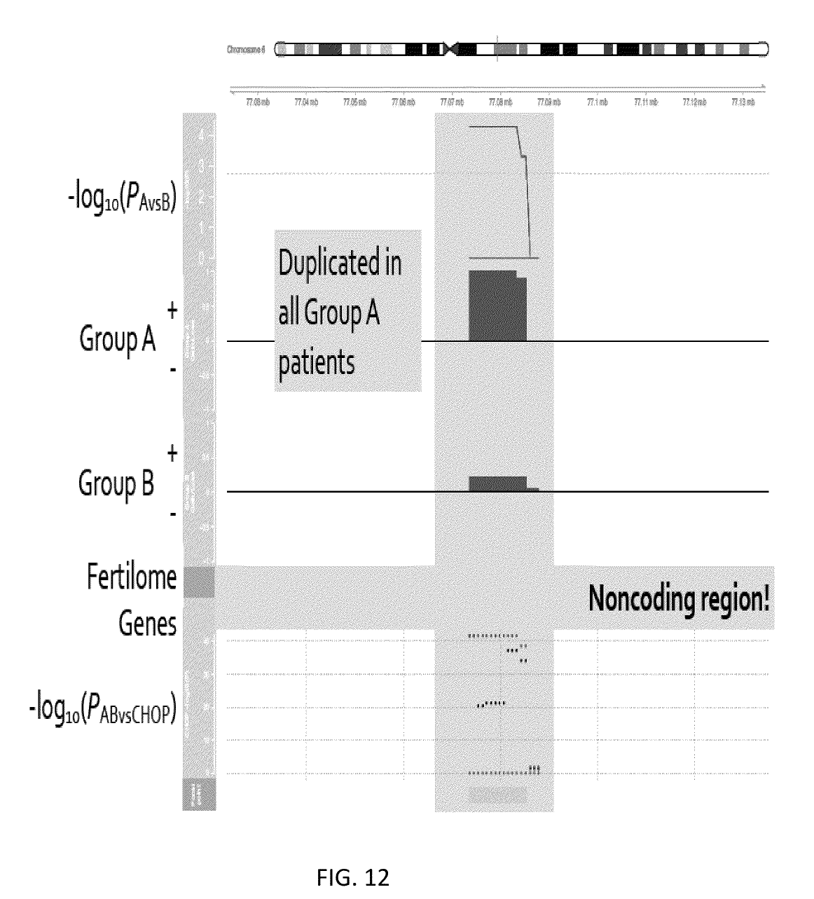

FIG. 9 provides CGH array data of copy number variations detected in the study populations within statistically significant regions associated with infertility (i.e., copy number variations within the Fertilome nucleic acid). FIG. 10 illustrates a specific copy number variation detected in the GJC2 gene of Chromosome 1 within Groups A and B. This region is specifically expressed in both the oocyte and brain, and is known to be associated with embryo issues. As shown, the region within GJC2 showed deletion in the most infertile patients. FIG. 11 illustrates a specific copy number variation detected in the CRTC1 and GDF1 genes of Chromosome 19 within Groups A and B. CRTC1 is associated with ovary, oocyte, endometrium, and placenta expression. GDF1 is associated with defects in the formation of anterior visceral endoderm and mesoderm. As shown, both patient groups exhibit copy number deletions in those genes. FIG. 12 illustrates a specific copy number variation detected in a non-coding region of Chromosome 6. As shown, both patient groups exhibit copy number duplication that region.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for assessing whether a genetic region is associated with infertility, the method comprising:
identifying a genetic region whose function is suspected of being associated with infertility;
producing a genetically modified mouse in which the genetic region whose function is suspected of being associated with infertility is altered; and
assessing the mouse for presence of an infertility-associated phenotype, wherein the presence of the phenotype is indicative of the genetic region being associated with infertility.

2. The method according to claim 1, wherein the genetic region comprises a gene.

3. The method according to claim 2, wherein identifying comprises:
obtaining data on a set of genetic loci, the set comprising genetic loci known to be associated with infertility and genetic loci having no prior association with infertility; and
performing a clustering analysis on the data to identify the genetic loci that have no prior association with infertility that cluster with one or more genetic loci known to be associated with infertility, wherein a genetic loci that has no prior association with infertility that clusters with a genetic loci known to be associated with infertility is classified as a being associated with infertility.

4. The method according to claim 1, wherein data is selected from the group consisting of: gene expression data, phenotype, knowledge of gene pathway, and any combination thereof.

5. The method according to claim 1, wherein the method further comprises:
administering a therapeutic agent to the mouse; and
assessing the effect of the therapeutic agent on the phenotype.

6. The method according to claim 1, wherein presence of the infertility-associated phenotype is used as a factor in ranking the importance of the gene in a database of genetic loci associated with infertility in humans.

7. The method according to claim 6, wherein presence of the phenotype increases the rank of the gene in the database.

8. The method according to claim 6, wherein absence of the phenotype decreases the rank of the gene in the database.

9. The method according to claim 1, wherein the alteration to the genetic region is a mutation.

10. The method according to claim 9, wherein the mutation is selected from the group consisting of: a single nucleotide polymorphism, a deletion, an insertion, a rearrangement, a copy number variation, and a combination thereof.

11. A method for assessing whether a human genetic alteration is associated with an infertility phenotype in a mouse, the method comprising:
identifying a human genetic region whose function is known to be associated with human infertility;
producing a genetically modified mouse in which the genetic region whose function is associated with human infertility is altered; and
assessing the mouse for presence of the infertility phenotype.

12. The method according to claim 11, wherein the genetic region comprises a gene.

13. The method according to claim 11, wherein the method further comprises:
administering a therapeutic agent to the mouse; and
assessing the effect of the therapeutic agent on the phenotype.

14. The method according to claim 11, wherein presence of the infertility phenotype is used as a factor is ranking an importance of the gene in a database of genetic loci associated with infertility in humans.

15. The method according to claim 14, wherein presence of the phenotype in the mouse increases the rank of the gene in the database.

16. The method according to claim 14, wherein absence of the phenotype in the mouse decreases the rank of the gene in the database.

17. The method according to claim 11, wherein the alteration to the genetic region is a mutation.

18. The method according to claim 17, wherein the mutation is selected from the group consisting of: a single nucleotide polymorphism, a deletion, an insertion, a rearrangement, a copy number variation, and a combination thereof.
